(19) United States
(12) Patent Application Publication
(10) Pub. No.: US 2025/0215055 A1
(43) Pub. Date: Jul. 3, 2025

(54) SECRETION-OPTIMIZED DE NOVO DESIGNED PROTEIN NANOPARTICLES FOR EUKARYOTIC EXPRESSION AND GENETIC DELIVERY

(71) Applicant: UNIVERSITY OF WASHINGTON, Seattle, WA (US)

(72) Inventors: Neil P. KING, Seattle, WA (US); Jing Yang WANG, Seattle, WA (US); Alena KHMELINSKAIA, Seattle, WA (US); Yang HSIA, Seattle, WA (US); David BAKER, Seattle, WA (US); Grace G. HENDRICKS, Seattle, WA (US); Daniel ELLIS, Seattle, WA (US)

(21) Appl. No.: 18/853,519

(22) PCT Filed: Apr. 5, 2023
(86) PCT No.: PCT/US2023/065397

§ 371 Date: Oct. 2, 2024

Related U.S. Application Data
(60) Provisional application No. 63/328,394, filed on Apr. 7, 2022.

(51) Int. Cl. C07K 14/005; A61K 39/00; C07K 14/00

(52) U.S. Cl. CPC C07K 14/005; C07K 14/001; A61K 39/00; C07K 2319/00; C12N 2770/20022; C12N 2770/20034

(57) ABSTRACT

Polypeptides having an amino acid sequence at least 50% identical to, and identical at least at one identified interface position, to the amino acid sequence selected from the group consisting of SEQ ID NO: 1-44, and polypeptides having an amino acid sequence at least 50% identical to the amino acid sequence selected from the group consisting of SEQ ID NO: 45-58, are provided, as well as fusion proteins thereof, nanoparticles thereof, and methods for treating or limiting development of an infection.

### CROSS REFERENCE

This application claims priority to U.S. Provisional Application Ser. No. 63/328,394 filed Apr. 7, 2022, incorporated by reference herein in its entirety.

### FEDERAL FUNDING STATEMENT

This invention was made with government support under Grant No. HDTRA1-18-1-0001, awarded by the Defense Threat Reduction Agency. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Mar. 30, 2023 having the file name “21-1319-WO.xml” and is 168,885 bytes in size.

### SUMMARY

In one aspect, the disclosure provides polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to, and identical at least at one identified interface position, to the amino acid sequence selected from the group consisting of SEQ ID NO: 1-44, wherein residues in parentheses are optional, and may be present or absent; wherein any N-terminal methionine residues are optional and may be present or absent; and wherein some or all of the optional residues may be absent and not included for determining percent identity.

In another aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:45-58, wherein residues in parentheses are optional, and may be present or absent; wherein any N-terminal methionine residues are optional and may be present or absent; wherein some or all of the optional residues may be absent and not included for determining percent identity.

In one embodiment, the disclosure provides fusion proteins, comprising:
(a) the polypeptide of any embodiment or combination of embodiments herein;
(b) one or more additional polypeptides; and
(c) optional amino acid linkers between the polypeptide and the one or more additional polypeptides.

In one embodiment, the one or more additional polypeptides comprise an antigen, including but not limited to a bacterial or viral antigen.

In other embodiments, the disclosure provides nucleic acids encoding the polypeptide or fusion protein of any embodiment or combination of embodiments herein; expression vectors comprising a nucleic acid of the disclosure operatively linked to a suitable control sequence; and host cells comprising the polypeptide, fusion protein, nucleic acid, or expression vector of any embodiment or combination of embodiments herein.

In one embodiment, the disclosure provides nanoparticles comprising a plurality of the polypeptides and/or the fusion proteins of any embodiment or combination of embodiments herein. In one embodiment, some or all the polypeptides or fusion proteins are fused to a polypeptide antigen, wherein the polypeptide antigen may be identical in all of the polypeptides or fusion proteins, or wherein the nanoparticle may present more than one polypeptide antigen.

The disclosure also provides pharmaceutical composition comprising
(a) the polypeptide, fusion protein, nucleic acid, cell, and/or nanoparticle of any embodiment or combination of embodiments herein; and
(b) a pharmaceutically acceptable carrier.

In one embodiment, the disclosure provides vaccines comprising
(a) the polypeptide, fusion protein, nucleic acid, cell, and/or nanoparticle of any embodiment or combination of embodiments herein involving an antigen or encoded antigen; and
(b) a pharmaceutically acceptable carrier.

In a further embodiment, the disclosure provides method for treating an infection, limiting development of an infection, and/or generating an immune response in a subject, comprising administering to an infected subject an amount effective to treat the infection of the fusion protein of the disclosure comprising an antigen, a nucleic acid encoding the fusion protein, an expression vector comprising the nucleic acid, a cell comprising the fusion protein, nucleic acid, or expression vector; and/or a pharmaceutical composition comprising the fusion protein, nucleic acid, expression vector, or cell.

### DESCRIPTION OF THE FIGURES

FIG. 1. Incorporation of the Degreaser prospectively during design to generate de novo designed secreted protein assemblies. (a) Trimeric building blocks were docked into a desired geometry: tetrahedral, octahedral, or icosahedral. For the KWOCAs, designs were run independently for DG and OG sets, while ND designs were selected from a filtered subset of all OG designs. (b) Expression and secretion characterization of KWOCAs shows the benefit of the Degreaser on secreted yield (positive expression in mammalian cells determined as greater secretion than 13-01). Assemblies validated by nsEM are highlighted in darker color. (c) nsEM-verified assembling (FIG. 6) secreted proteins partitioned into OG, ND, and DG groups show the enhanced secreted yield of DG designs. (d) Constructs purified from mammalian material assembled into well-defined particles, indistinguishable from those expressed in bacteria (FIG. 6). Scale bar, 100 nm. (e) Left, representative western blot of KWOCAs with lowest dG_{ins,pred} and secretion yield (K0 and K47) and highest secretion yield (K100 and K101). Right, quantification of secreted yield, measured in triplicate.

FIG. 2. Structural characterization of KWOCA 4 and KWOCA 51. (a) DLS, SEC traces and (b) SAXS profiles of KWOCA 51 (blue) and KWOCA 4 (red/orange). (c) Design model and cryo-EM density map of KWOCA 51. (d) Cryo-EM density map of KWOCA 4. (e) Overlay of two KWOCA 4 subunits across the designed nanoparticle interface, highlighting the interface contact angle difference between the design model and the best-fitting cryo-EM model. Theoretical SAXS profiles calculated from the design models (dotted darker lines) are overlaid with the experimentally obtained SAXS profiles (b). Scale bar, 5 nm (c,d).

FIG. 3. Confirmation of 13 of 22 potentially assembling KWOCAs by nsEM. Constructs purified both from bacterial and mammalian material (second and third rows) assembled into indistinguishable particles. Scale bar, 100 nm.

FIG. 4. SEC and crystal structure of a non-assembling KWOCA. (a) SEC profile and (b) crystal structure of the non-assembling KWOCA 39. The structure is aligned to the backbone of a single subunit of the computational design model, and rmsd values for the monomer and trimer are shown.

FIG. 5. Confirmation of assembly of 5 antigen-bearing secretion-optimized nanoparticles by nsEM. Constructs purified from mammalian material assembled into indistinguishable particles. (a) Rpk9_RBD_SARS-COV-2_13-01-NS, (b) Rpk9_RBD_SARS-CoV-2_KWOCA-51, (c) Rpk9_RBD_SARS-COV-2_KWOCA-101, (d) Rpk9_RBD_SARS-CoV-2 KWOCA-18, and (e) Rpk9_RBD_SARS-COV-2 KWOCA-4. Scale bars are 100 nm (a, b, c) or 200 nm (d, e).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In any polypeptide disclosed herein, any N-terminal methionine residue is optional and may be present or may be deleted.

As used herein, “about” means +/−5% of the recited parameter.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical, and identical at least at one identified interface position, to the amino acid sequence selected from the group consisting of SEQ ID NO: 1-44, wherein residues in parentheses (as shown in Tables 1 and 2) are optional, and may be present or absent; wherein any N-terminal methionine residues are optional and may be present or absent; and wherein some or all of the optional residues may be absent and not included for determining percent identity.

The isolated polypeptides of this embodiment can be used, for example, as scaffolds for vaccines or signaling receptor agonists. The polypeptides based on the Table 1 and Table 2 examples form trimeric building blocks that assemble to form nanoparticles (i.e.: particles having a widest dimension between 1-999 nm). The interface residues for each reference polypeptide identified in Tables 1-2 are those at the interface between trimeric building blocks. The tables provides the amino acid sequence of exemplary polypeptides of the disclosure; the right hand column in the tables identifies the residue numbers in each exemplary polypeptide that were identified as present to the interface of resulting assembled nanostructures (i.e.: “identified interface residues”). As can be seen in Tables 1 and 2, the number of interface residues for the exemplary polypeptides varies between different polypeptides. In various embodiments, the isolated polypeptides are identical at least at 1, 2, 3, 4, 5, 6, 7, 8, 9, 10, 11, 12, 13, 14, 15, 16, 17, 18, 19, 20, 21, 22, 23, 24, 25, more, or all identified interface positions.

Residue numbering as shown in Tables 1-2 is based on residue number I being the first non-optional residue listed (i.e.: the first residue not in parentheses). For each reference polypeptide, the sequence of a bacterially-expressed embodiment and a mammalian cell-expressed embodiment are shown.

TABLE 1

| | | | | interface |
|---|---|---|---|---|
| | | | | residues |
| | | | | Residue 1 |
| | | | | is first |
| | | | | non- |
| KWOCA | Other | Bacterially-expressed | Mammalian-expressed | optional |
| # | name | sequence | sequence | residue |
| 4 | I3_HF_ | (MHHHHHHGGSEQKLISEEDLSG | (METDTLLLWVLLLWVPGSTGDGSHH | 8, 11, 12, 13, |
| | OG_04 | GGSWSGS)TEVEKKAREVAKEAV | HHHHGGSEQKLISEEDLSGGGSWSGS) | 15, 16, 18, |
| | | ELASLERSETAIRVAQAILEAAE | TEVEKKAREVAKEAVELASLLRSET | 19, 20, 21, |
| | | AAKRAAEQGKTEVAKLALKVLEE | AIRVAQAILEAAEAAKRAAEQGKTEV | 22, 23, 24, 25, |
| | | AIELAKEKRSEEALKVVLEIARA | AKLALKVLEEAIELAKEKRSEEALKV | 27, 28, 30, |
| | | ALAAAQAAEEGFTDVAKMALEVL | VLEIARAALAAAQAAEEGFTDVAKMA | 31, 32, 34, |
| | | ERAIELAKDDRSEEALKEVLEIA | LEVLERAIELAKDDRSEEALKEVLEI | 35, 38 |
| | | RAALAAAQLAKKGRDDEARKILM | ARAALAAAQLAKKGRDDEARKILMKL | |
| | | KLRIRITLRKLEESLRELRRILE | RIRITLRKLEESLRELRRILEELKEM | |
| | | ELKEMLERLEKNPDKDVIVKVLK | LERLEKNPDKDVIVKVLKVIVKAIEA | |
| | | VIVKAIEASVENQRISAENQKAL | SVENQRISAENQKALAELA** | |
| | | AELA** | SEQ ID NO: 2 | |
| | | SEQ ID NO: 1 | | |
| 18 | I3_HF_ | (MHHHHHHGGSEQKLISEEDLSG | (METDTLLLWVLLLWVPGSTGDGSHH | 11, 14, 15, 18, |
| | OG_18 | GGSWSGS)SDEEEAREWAERALK | HHHHGGSEQKLISEEDLSGGGSWSGS) | 21, 22, 25, |
| | | AALEAAEQALREGDEDAFKCAVE | SDEEEAREWAERALKAALEAAEQAL | 30, 33, 34, |
| | | LLEQALEARKKKDSEEAEAVYWA | REGDEDAFKCAVELLEQALEARKKKD | 37, 38, 40, 41, |
| | | ARAVLAALEALEQAKREGDEDAR | SEEAEAVYWAARAVLAALEALEQAKR | 44, 47, 48 |
| | | RCAEELLRLACEAARKKNSEQAR | EGDEDARRCAEELLRLACEAARKKNS | |
| | | AVYEAARAVLAALRALEAAKRAG | EQARAVYEAARAVLAALRALEAAKRA | |
| | | MEEARKEAEELLRRACEAARKQD | GMEEARKEAEELLRRACEAARKQDPE | |
| | | PELARAVRDKAELLKALADLFKA | LARAVRDKAELLKALADLFKALKELK | |
| | | LKELKKSLDELERSLEELEKNPS | KSLDELERSLEELEKNPSEDALVENN | |
| | | EDALVENNRLNVENNKIIVEVLR | RLNVENNKIIVEVLRIIAEVLRINAR | |
| | | IIAEVLRINARAV** | AV** | |
| | | SEQ ID NO: 3 | SEQ ID NO: 4 | |
| 46 | T3_HF_ | (MHHHHHHGGSEQKLISEEDLSG | (METDTLLLWVLLLWVPGSTGDGSHH | 7, 11, 14, 18, |
| | OG_01 | GGSWSGS)TEEKIEEARQAIKEA | HHHHGGSEQKLISEEDLSGGGSWSGS) | 21, 23, 25, |
| | | ERALREGDPRFAEMAVRIALALV | TEEKIEEARQAIKEAERALREGDPR | 26, 28, 29, |
| | | RMLERLARKTGSTEVLIEAARLA | FAEMAVRIALALVRMLERLARKTGST | 30, 32, 33, 35, |
| | | IEVARVALKVGSPETAREAVRTA | EVLIEAARLAIEVARVALKVGSPETA | 36, 37, 39, |
| | | LELVQELERQARKTGSTEVLIEA | REAVRTALELVQELERQARKTGSTEV | 40, 43, 44, 47 |
| | | ARLAIEVARVAFKVGSPETAKEA | LIEAARLAIEVARVAFKVGSPETAKE | |
| | | VRTALELVKELIQQALKTGSDEV | AVRTALELVKELIQQALKTGSDEVLE | |
| | | LERAAELAKEVARVAKEVGDPRA | RAAELAKEVARVAKEVGDPRAARKAD | |
| | | ARKADMVAKIADTLRELMESTRE | MVAKIADTLRELMESLRELRRILEEL | |
| | | LRRILEELKEMLERLEKNPDKDV | KEMLERLEKNPDKDVIVKVLKVIVKA | |
| | | IVKVLKVIVKAIEASVENQRISA | IEASVENQRISAENQAALASLA** | |
| | | ENQAALASLA** | SEQ ID NO: 6 | |
| | | SEQ ID NO: 5 | | |
| 47 | T3_HF_ | (M)ADELRAVAELQRENIELARK | (METDTLLLWVLLLWVPGSTGDGS)A | 165, 173, 215, |
| | OG_02 | LLEAVARLQELNIDLVRKTSELT | DELRAVAELQRLNIELARKLLEAVAR | 218, 219, |
| | | DEKTIREEIRKVKEESKRIVEEA | LQELNIDLVRKTSELTDEKTIREEIR | 220, 222, 223, |
| | | EEEIRRAKEDSKRIVTEALRRAR | KVKEESKRIVEEAEEEIRRAKEDSKR | 224, 226, |
| | | EQIREKWEELEERAKRAETPEEA | IVTEALRRAREQIREKWEELEERAKR | 227, 228, 273, |
| | | LRAAEEMVKLIEELIRIAEMLQR | AETPEEALRAAEEMVKLIEELIRIAE | 276, 277, |
| | | AGLKEEAEDVLREATELIKRATE | MLQRAGLKEEAEDVEREATELIKRAT | 280, 284, 287 |
| | | LLEKIAKNSDTPELALRAAELLV | ELLEKIAKNSDTPELALRAAELLVRL | |
| | | RLIKLLIEIAKLLQEQGNKEEAE | IKLLIEIAKLLQEQGNKEEAEKVLRE | |
| | | KVLREATELIKRVARLLLAIALL | ATELIKRVARLLLAIALLADTPELAK | |
| | | ADTPELAKRAAELLKRLIELLKE | RAAELLKRLIELLKEIAKLLEEEGNE | |
| | | IAKLLEEEGNEDEAEKVKEEAKE | DEAEKVKEEAKELEELVRWLEEQIRG | |
| | | LEELVRWLEEQIRG(GSWSGGGS | (GSWSGGGSEQKLISEEDIGGS) | |
| | | EQKLISEEDIGGS) | SEQ ID NO: 8 | |
| | | SEQ ID NO: 7 | | |
| 51 | T3_HF_ | (MHHHHHHGGSEQKLISEEDLSG | (METDTLLLWVLLLWVPGSTGDGSHH | 30, 31, 33, |
| | OG_06 | GGSWSGS)SDEEEAREWAERAEE | HHHHGGSEQKLISEEDLSGGGSWSGS | 34, 37, 38, 40, |
| | | AAKEALEQAKREGDEIARLCAKM | )SDEEEAREWAERAEEAAKEALEQAK | 41, 42, 45, |
| | | LEILAEEARRKKDSEEAEAVYWA | REGDEIARLCAKMLEILAEEARRKKD | 91, 92, 93, 96, |
| | | ARAVLAALEALEQAKREGDEDAR | SEEAEAVYWAARAVLAALEALEQAKR | 99, 100, 101, |
| | | RCAEELLRLACSAAARQDSEQAR | EGDEDARRCAEELLRLACSAAARQDS | 102 |
| | | AVYEAARAVLAALRALEAAKRAG | EQARAVYEAARAVLAALRALEAAKRA | |
| | | MEEARKEAEELLRRACEAARKQD | GMEEARKEAEELLRRACEAARKQDPE | |
| | | PELARAVRDKAELLKALADLFKA | LARAVRDKAELLKALADLFKALKELK | |
| | | LKELKKSLDELERSLEELEKNPS | KSLDELERSLEELEKNPSEDALVENN | |
| | | EDALVENNRENVENNKIIVEVLR | RLNVENNKIIVEVLRIIAEVLRINAR | |
| | | IIAEVERINARAV** | AV** | |
| | | SEQ ID NO: 9 | SEQ ID NO: 10 | |
| 58 | I3_HF_ | (M)DECERLETEVMKAAKELMKL | (METDTLLLWVLLLWVPGSTGDGS)D | 4, 7, 8, 11, |
| | ND_02 | ATQSTDKEVRKIAWEVADQLLRL | ECERLETEVMKAAKELMKLATQSTDK | 14, 15, 18, 19, |
| | | AEEACRSNSDECLRLASEVVKAV | EVRKIAWEVADQLLRLAEEACRSNSD | 21, 22, 27, |
| | | QELVKLAEQATDEEVIRVALEVA | ECLRLASEVVKAVQELVKLAEQATDE | 30, 31, 34, 35, |
| | | RELIRLAQEACRSNDDECERLAS | EVIRVALEVARELIRLAQEACRSNDD | 38, 41, 45 |
| | | EVVKAVQEAVKLAEQAKDERVIE | ECLRLASEVVKAVQEAVKLAEQAKDE | |
| | | VALEMARLLIELAQEACRRNDEE | RVIEVALEMARLLIELAQEACRRNDE | |
| | | ALRRASEIVKRVQELIKEAEKAT | EALRRASEIVKRVQELIKEAEKATDE | |
| | | DEEEIERLLRKAAIDITLAQLEI | EEIERLLRKAAIDITLAQLEISLKEL | |
| | | SLKELRRILEELKEMLERLEKNP | RRILEELKEMLERLEKNPDKDVIVKV | |
| | | DKDVIVKVLKVIVKAIEASVENQ | LKVIVKAIEASVENQRISAENQKMLA | |
| | | RISAENQKMLAELA(GSWSGGGS | ELA(GSWSGGGSEQKLISEEDIGGS) | |
| | | EQKLISEEDIGGS) | SEQ ID NO: 12 | |
| | | SEQ ID NO: 11 | | |
| 67 | I3_HF_ | (MHHHHHHGGSEQKLISEEDLSG | (METDTLLLWVLLLWVPGSTGDGSHH | 15, 19, 22, |
| | ND_11 | GGSWSGS)TEEKIAKEISRIAEE | HHHHGGSEQKLISEEDLSGGGSWSGS) | 23, 26, 29, 30, |
| | | SKKAIETLARLADKMTDENQVDT | TEEKIAKEISRIAEESKKAIETLAR | 31, 32, 34, |
| | | AIELIAKIAREAIKRIEDLAKNL | LADKMTDENQVDTAIELIAKIAREAI | 35, 38, 39, 41, |
| | | ASEEFMARAISAIAELAKKAIEA | KRIEDLAKNLASEEFMARAISAIAEL | 42, 45, 46, |
| | | IYRLAELHRTDTFMAKAIEAIAE | AKKAIEAIYRLAELHRTDTEMAKAIE | 90, 91, 93, |
| | | LAKEAIKAIADLAKKHTTEEFMA | AIAELAKEAIKAIADLAKKHTTEEFM | 149, 152, 153, |
| | | RAISAIAELAKKAIEAIWRLASL | ARAISAIAELAKKAIEAIWRLASLHK | 155, 207, |
| | | HKTDEFMDKAAEAIAELAEEAIR | TDEFMDKAAEAIAELAEEAIRAIRED | 211, 214, 215, |
| | | AIRELAKKHTTEEFVRKAESAVR | AKKHTTEEFVRKAESAVREISKKAKD | 217 |
| | | EISKKAKDAIRKLADAMRDPTAR | AIRKLADAMRDPTAREKAKKLEIKVE | |
| | | EKAKKLEIKVELAEALAELAVAL | LAEALAELAVALLKLKLSLDELERSL | |
| | | LKLKLSLDELERSLEELEKNPSE | EELEKNPSEDALVENNRLNVENNKII | |
| | | DALVENNRINVENNKIIVEVLRI | VEVLRIIAEVLDINAQLV** | |
| | | IAEVLDINAQLV** | SEQ ID NO: 14 | |
| | | SEQ ID NO: 13 | | |
| 70 | O3_HF_ | (M)DESVDLAVKLAEALRKEAEE | (METDTLLLWVLLLWVPGSTGDGS)D | 1, 2, 3, 4, 5, |
| | ND_02 | LIKKARKTGDPELLRKALEALEK | ESVDLAVKLAEALRKEAEELIKKARK | 6, 7, 9, 10, |
| | | AVKLVEDAIKRNPDNDEAVETAV | TGDPELLRKALEALEKAVKLVEDAIK | 12, 13, 14, 15, |
| | | RLARELKKVAEELQERAKKTGDP | RNPDNDEAVETAVRLARELKKVAEEL | 16, 17, 43, |
| | | ELLKLALRALEVAVRAVELAIKS | QERAKKTGDPELLKLALRALEVAVRA | 47, 50, 54 |
| | | NPDNDEAVKTAVELAKELEKVAR | VELAIKSNPDNDEAVKTAVELAKELE | |
| | | ELLERARKTGDDELLKLAKRALE | KVARELLERARKTGDDELLKLAKRAL | |
| | | VARRAVELALKSRPDAEEARRVY | EVARRAVELALKSRPDAEEARRVYIR | |
| | | IRLTEMELEISLTELRKILEELK | LTEMELEISLTELRKILEELKEMLER | |
| | | EMLERLEKNPDKDVIVKVLKVIV | LEKNPDKDVIVKVLKVIVKAIEASVE | |
| | | KAIEASVENQRISAENQKALAEL | NQRISAENQKALAELA(GSWSGGGSE | |
| | | A(GSWSGGGSEQKLISEEDIGGS) | QKLISEEDIGGS) | |
| | | SEQ ID NO: 15 | SEQ ID NO: 16 | |
| 75 | 03_HF_ | (MHHHHHHGGSEQKLISEEDLSG | (METDTLLLWVLLLWVPGSTGDGSHH | 1, 2, 5, 6, 9, |
| | ND_07 | GGSWSGS)DEEVWKAVIDAIELM | HHHHGGSEQKLISEEDLSGGGSWSGS) | 12, 13, 15, 16, |
| | | KEARELIKKARKTGDPELLRKAL | DEEVWKAVIDAIELMKEARELIKKA | 19, 20, 59, |
| | | EALEEAVRAVEEAIKRNPDNKIA | RKTGDPELLRKALEALEEAVRAVEEA | 60, 63, 64, |
| | | VIVAVLLARELKKVAEELQERAK | IKRNPDNKIAVIVAVLLARELKKVAE | 67, 118 |
| | | KTGDPELLKLALRALEVAVRAVE | ELQERAKKTGDPELLKLALRALEVAV | |
| | | LAIKSNPDNDEAVETAVRLAEEL | RAVELAIKSNPDNDEAVETAVRLAEE | |
| | | AKVAKELIERAKKTGDADLERLA | LAKVAKELIERAKKTGDADLLRLAKR | |
| | | KRAIEVARRAVELAKKSRPDAER | AIEVARRAVELAKKSRPDAERADEAY | |
| | | ADEAYKRLKELEREIRELLRKML | KRLKELEREIRELLRKMLTEALRKLE | |
| | | TEALRKLEKALQELREMLRKLKE | KALQELREMLRKLKESLEELKKNPSE | |
| | | SLEELKKNPSEDALVRNNELIVE | DALVRNNELIVEVLRVIVEVLSIIAE | |
| | | VLRVIVEVLSIIAEVLKINAALV | VLKINAALV** | |
| | | ** | SEQ ID NO: 18 | |
| | | SEQ ID NO: 17 | | |
| 100 | T3_HF_ | (MHHHHHHGGSEQKLISEEDLSG | (METDTLLLWVLLLWVPGSTGDGSHH | 7, 11, 14, 18, |
| | DG_01 | GGSWSGS)TEETIEMARQLIKEA | HHHHGGSEQKLISEEDLSGGGSWSGS) | 21, 23, 25, |
| | | ERALREGDPEEARMAVEMALAAV | TEETIEMARQLIKEAERALREGDPE | 26, 28, 29, 30, |
| | | RILERQARKTGSTEVLIEAARLA | EARMAVEMALAAVRILERQARKTGST | 32, 33, 35, |
| | | IEVARVALKVGSPETAREAVRTA | EVLIEAARLAIEVARVALKVGSPETA | 36, 37, 39, |
| | | LELVQELERQARKTGSTEVLIEA | REAVRTALELVQELERQARKTGSTEV | 40, 43, 44, 47 |
| | | ARLAIEVARVAFKVGSPETAKEA | LIEAARLAIEVARVAFKVGSPETAKE | |
| | | VRTALELVKELIQQALKTGSDEV | AVRTALELVKELIQQALKTGSDEVLE | |
| | | LERAAELAKEVARVAKEVGDPRA | RAAELAKEVARVAKEVGDPRAARKAD | |
| | | ARKADMVAKIADTLRELMESIRE | MVAKIADTERELMESTRELRRILEEL | |
| | | LRRILEELKEMLERLEKNPDKDV | KEMLERLEKNPDKDVIVKVLKVIVKA | |
| | | IVKVLKVIVKAIEASVENQRISA | IEASVENQRISAENQAALASLA** | |
| | | ENQAALASLA** | SEQ ID NO: 20 | |
| | | SEQ ID NO: 19 | | |
| 101 | T3_HF_ | (MHHHHHHGGSEQKLISEEDLSG | (METDTLLLWVLLLWVPGSTGDGSHH | 30, 31, 33, 34, |
| | DG_02 | GGSWSGS)SDEEEAREWAERAEE | HHHHGGSEQKLISEEDLSGGGSWSGS) | 37, 38, 40, |
| | | AAKEALEQAKREGDEIARLCAEM | SDEEEAREWAERAEEAAKEALEQAK | 41, 42, 45, |
| | | LEILAEEARRKKDSEEAEAVYWA | REGDEIARLCAEMLEILAEEARRKKD | 91, 92, 93, 96, |
| | | ARATLAALEALEQAKREGDEDAR | SEEAEAVYWAARATLAALEALEQAKR | 99, 100, 101, |
| | | RCAEELLRLACSAAARQDSEQAR | EGDEDARRCAEELLRLACSAAARQDS | 102 |
| | | AVYEAARAVLAALRALEAAKRAG | EQARAVYEAARAVLAALRALEAAKRA | |
| | | MEEARKEAEELLRRACEAARKQD | GMEEARKEAEELLRRACEAARKQDPE | |
| | | PELARAVRDKAELLKALADLFKA | LARAVRDKAELLKALADLFKALKELK | |
| | | LKELKKSIDELERSLEELEKNPS | KSLDELERSLEELEKNPSEDALVENN | |
| | | EDALVENNRINVENNKIIVEVLR | RLNVENNKIIVEVLRIIAEVLRINAR | |
| | | IIAEVLERINARAV** | AV** | |
| | | SEQ ID NO: 21 | SEQ ID NO: 22 | |
| 102 | T3_HF_ | (M)MTEEKIEEARQSIKEAERSL | (METDTLLLWVLLLWVPGSTGDGS)T | 25, 26, 28, 29, |
| | DG_03 | REGNPEKALDAVARALSLVNELE | EEKIEEARQSIKEAERSLREGNPEKA | 30, 32, 33, |
| | | RLARKTGSTEVLIEAARLAIEVA | LDAVARALSLVNELERLARKTGSTEV | 35, 36, 37, |
| | | RVALKVGSPEMAQLAVELALRLV | LIEAARLAIEVARVALKVGSPEMAQL | 39, 40, 42, 43, |
| | | QELERQARKTGSTEVLIEAARLA | AVELALRLVQELERQARKTGSTEVLI | 74, 75, 76, |
| | | IEVARVAFKVGSPETAREAARTA | EAARLAIEVARVAFKVGSPETAREAA | 78, 79, 82, |
| | | LELVEELERQARKTGSEEVLERA | RTALELVEELERQARKTGSEEVLERA | 83 |
| | | ARLAEEVARVAEEIGDPELARKA | ARLAEEVARVAEEIGDPELARKAMKV | |
| | | MKVAIRLTEELLKKSLRELRRIL | AIRLTEELLKKSLRELRRILEELKEM | |
| | | EELKEMLERLEKNPDKDVIVKVL | LERLEKNPDKDVIVKVLKVIVKAIEA | |
| | | KVIVKAIEASVENQRISADNQRA | SVENQRISADNQRALARLA(GSWSGG | |
| | | LARLA(GSWSGGGSEQKLISEED | GSEQKLISEEDLGGS) | |
| | | LGGS) | SEQ ID NO: 24 | |
| | | SEQ ID NO: 23 | | |
| 0 | I3_HF_ | (M)SDEVDRRVLELAIKASRATD | (METDTLLLWVLLLWVPGSTGDGS)S | 2, 5, 6, 9, 10, |
| | OG_00 | KEEVIEIVKELAELAKQSTDSRL | DEVDRRVLELAIKASRATDKEEVIEI | 13, 16, 17, |
| | | VERIVTLLALVAIDATDKELVIY | VKELAELAKQSTDSRLVERIVTLLAL | 42, 45, 46, |
| | | IVKILAELAKQSTDSELVKKIVE | VAIDATDKELVIYIVKILAELAKQST | 49, 50, 53, 56, |
| | | MLAQVARFATDKELVEYIARILL | DSELVKKIVEMLAQVARFATDKELVE | 57, 85, 86, |
| | | ELAKQADDATLVAFIAEMLAEVR | YIARILLELAKQADDATLVAFIAEML | 89, 90, 93, 96, |
| | | KEAKDKELKEKIDEILKELAKIT | AEVRKEAKDKELKEKIDEILKELAKI | 97, 126, 133 |
| | | LKALEDSLRELRRILEELKEMLE | TLKALEDSLRELRRILEELKEMLERL | |
| | | RLEKNPDKDVIVKVLKVIVKAIE | EKNPDKDVIVKVLKVIVKAIEASVKN | |
| | | ASVKNQEISAANQKALALLG(LE | QEISAANQKALALLG(GSEQKLISEE | |
| | | HHHHHH) | DL) | |
| | | SEQ ID NO: 25 | SEQ ID NO: 26 | |

TABLE 2

| | | | | interface |
|---|---|---|---|---|
| | | | | residues |
| | | | | Residue 1 |
| | | | | is the |
| | | | | first non- |
| KWOCA | Other | Bacterially-expressed | Mammalian-expressed | optional |
| # | name | sequence | sequence | residue |
| 60 | I3_HF_ | (M)IMSKIAETAKRLADSLRELR | (METDTLLLWVLLLWVPGSTGDGS) | 32, 36, 40, 41, |
| | ND_04 | RILEELKEMLERLEKRPDKKVIV | IMSKIAETAKRLADSLRELRRIL | 44, 232, 233, |
| | | DVLKVIVKAIEASVENQRISASN | EELKEMLERLEKRPDKKVIVDVLK | 235, 236, 238, |
| | | QAALALAIAAEAVKEIEEDIDRA | VIVKAIEASVENQRISASNQAALA | 239, 240, |
| | | RKLKDEGNKEEAEKVLRKAREKI | LAIAAEAVKEIEEDIDRARKLKDE | 242, 250, 252, |
| | | REVRDALDAIAKGAGTPDIALKA | GNKEEAEKVLRKAREKIREVRDAL | 254, 255, 289, |
| | | AELLVRLIKLLIEIAKLLQDAGN | DAIAKGAGTPDIALKAAELLVRLI | 290, 292, 293, |
| | | KEEAEKVLREATELIKRVTELLE | KLLIEIAKLLQDAGNKEEAEKVLR | 294, 296, |
| | | KIAKNSDTPELALRAAELLVRLI | EATELIKRVTELLEKIAKNSDTPE | 297, 298, 300, |
| | | KLLIEIAKLLQEQGNKEEAEKVL | LALRAAELLVRLIKLLIEIAKLLQ | 301, 302, 303, |
| | | REATKMIIRVAQLLVKIAKNSDE | EQGNKEEAEKVLREATKMIIRVAQ | 304, 305, 306, |
| | | PELAKRAAELLKRLIELLKEIAK | LIVKIAKNSDEPELAKRAAELLKR | 307 |
| | | LLEEEGNEDEAEKVKEIAKILEE | LIELLKEIAKLLEEEGNEDEAEKV | |
| | | AVRELEERIIG(GSWSGGGSEQK | KEIAKILEEAVRELEERIIG(GSW | |
| | | LISEEDLGGS) | SGGGSEQKLISEEDLGGS) | |
| | | SEQ ID NO: 27 | SEQ ID NO: 28 | |
| 64 | I3_HF_ | (M)AELAIEMARQSIREAERSLL | (METDTLLLWVLLLWVPGSTGDGS) | 1, 2, 3, 4, 7, |
| | ND_08 | EGNPEKAREDVRRALELVRLLEK | AELAIEMARQSIREAERSLLEGN | 13, 14, 16, 17, |
| | | IARREGSTEVLIEAARLAIEVAR | PEKAREDVRRALELVRLLEKIARR | 19, 20, 21, 37, |
| | | VALWVGSPETAREAVRTALELVQ | EGSTEVLIEAARLAIEVARVALWV | 40, 44, 47, 48, |
| | | ELERQARKTGSTEVLIEAARLAI | GSPETAREAVRTALELVQELERQA | 66, 70, 120, |
| | | EVARVAFEVGSPETAREAARTAL | RKTGSTEVLIEAARLAIEVARVAF | 209, 212, 213, |
| | | ELVEELDRQAEKTGSKEVLERAA | EVGSPETAREAARTALELVEELDR | 216, 219, |
| | | RLAKEVARVAKEIGDPELARKAD | QAEKTGSKEVLERAARLAKEVARV | 220, 224, 225, |
| | | EVAERLDIKRTLLDLEDSLRELR | AKEIGDPELARKADEVAERLDIKR | 227, 228 |
| | | RILEELKRALEMLEKLPDKEMIR | TLLDLEDSLRELRRILEELKRALE | |
| | | DVLKVIVKAIEASVENQRISAEN | MLEKLPDKEMIRDVLKVIVKAIEA | |
| | | QKALARLA(GSWSGGGSEQKLIS | SVENQRISAENQKALARLA(GSWS | |
| | | EEDIGGS) | GGGSEQKLISEEDLGGS) | |
| | | SEQ ID NO: 29 | SEQ ID NO: 30 | |
| 65 | I3_HF_ | (MHHHHHHGGSEQKLISEEDLSG | (METDTLLLWVLLLWVPGSTGDGS | 2, 5, 6, 9, 10, |
| | ND_09 | GGSWSGS)SKEVTERVAELAAEA | HHHHHHGGSEQKLISEEDLSGGGS | 13, 14, 16, 17, |
| | | VRATDKEEVIEIVKELAELAKQS | WSGS)SKEVTERVAELAAEAVRAT | 19, 46, 49, 50, |
| | | TDSELVNFIVRALAAVAIAAQDK | DKEEVIEIVKELAELAKQSTDSEL | 53, 56, 57, |
| | | ELVIYIVKILAELAKQSTDSELV | VNFIVRALAAVAIAAQDKELVIYI | 59, 90, 93, 96, |
| | | NEIVKALAEVAKAATDKELVKYI | VKILAELAKQSTDSELVNEIVKAL | 97 |
| | | VDILLELAKQADDATLVAKIAEQ | AEVAKAATDKELVKYIVDILLELA | |
| | | LAEVREEAKDKELQERIDRVLKK | KQADDATLVAKIAEQLAEVREEAK | |
| | | LIEITLRALEESLRELRRILEEL | DKELQERIDRVLKKLIEITLRALE | |
| | | KEMLERLEKNPDKDVIVKVLKVI | ESLRELRRILEELKEMLERLEKNP | |
| | | VKAIEASVRNQQISAANQKALAL | DKDVIVKVLKVIVKAIEASVRNQQ | |
| | | LA** | ISAANQKALALLA** | |
| | | SEQ ID NO: 31 | SEQ ID NO: 32 | |
| 69 | O3_HF_ | (M)SSEEAERIARILEEVWSPDP | (METDTLLLWVLLLWVPGSTGDGS) | 1, 2, 4, 5, 6, |
| | ND_01 | ENIREAVRKAEELLRENPSRQAE | SSEEAERIARILEEVWSPDPENI | 7, 8, 9, 10, 12, |
| | | ELLREAIEAAVRAPDPEAIREAV | REAVRKAEELLRENPSRQAEELLR | 13, 14, 15, 16, |
| | | RAAEELLRENPSTEAEELLRRAI | EAIEAAVRAPDPEAIREAVRAAEE | 17, 39, 40, 41, |
| | | EAAVRAPDPEAIREAVRAARELE | LLRENPSTEAEELLRRAIEAAVRA | 44, 48 |
| | | KENPSEEAEELLKRAADSALKAP | PDPEAIREAVRAARELFKENPSEE | |
| | | DPRAIREALEALLELLEAALRRL | AEELLKRAADSALKAPDPRAIREA | |
| | | KKSLDELERSLEELEKNPSEDAL | LEALLELLEAALRRLKKSLDELER | |
| | | VENNRLNVENNKIIVKVLEIIAR | SLEELEKNPSEDALVENNRLNVEN | |
| | | VLKANARLV(GSWSGGGSEQKLI | NKIIVKVLEIIARVLKANARLV(G | |
| | | SEEDLGGS) | SWSGGGSEQKLISEEDLGGS) | |
| | | SEQ ID NO: 33 | SEQ ID NO: 34 | |
| 73 | O3_HF_ | (M)ALEKDRRALEALKRAQEAEK | (METDTLLLWVLLLWVPGSTGDGS) | 1, 2, 3, 4, 5, |
| | ND_05 | KGDVEEAVRAAQEAVRAAKESGA | ALEKDRRALEALKRAQEAEKKGD | 6, 7, 9, 10, 13, |
| | | SWILRLVAEQALRIAKEAEKQGN | VEEAVRAAQEAVRAAKESGASWIL | 16, 43, 44, 45, |
| | | VEVAVKAARVAVEAAKQAGDNDV | RLVAEQALRIAKEAEKQGNVEVAV | 46, 48, 49 |
| | | LRKVAEQALRIAKEAEKQGNVDV | KAARVAVEAAKQAGDNDVLRKVAE | |
| | | AAKAAQVAAEAAKQAGDKDMLEK | QALRIAKEAEKQGNVDVAAKAAQV | |
| | | VAKVAEQIAKAAEKEGDKKVSID | AAEAAKQAGDKDMLEKVAKVAEQI | |
| | | ATRIALEASLAALEIILEELKEM | AKAAEKEGDKKVSIDATRIALEAS | |
| | | LERLEKNPDKDVIVKVLKVIVKA | LAALEIILEELKEMLERLEKNPDK | |
| | | IEASVKNQKISAKNQKALAELA | DVIVKVLKVIVKAIEASVKNQKIS | |
| | | (GSWSGGGSEQKLISEEDLGGS) | AKNQKALAELA(GSWSGGGSEQKL | |
| | | SEQ ID NO: 35 | ISEEDIGGS) | |
| | | | SEQ ID NO: 36 | |
| 77 | T3_HF_ | (MHHHHHHGGSEQKLISEEDLSG | (METDTLLLWVLLLWVPGSTGDGS | 30, 31, 32, 33, |
| | ND_02 | GGSWSGS)PRERLEEAKERVEEI | HHHHHHGGSEQKLISEEDLSGGGS | 35, 36, 37, 39, |
| | | RELIDKARKLQEQGDRIRATAVL | WSGS)PRERLEEAKERVEEIRELI | 40, 42, 43, |
| | | MEARAQIEEVTRELEEIAKNSDT | DKARKLQEQGDRIRATAVLMEARA | 46, 91, 92, 93, |
| | | PELALRAAELLVRLIKLLIEIAK | QIEEVTRELEEIAKNSDTPELALR | 94, 96, 97, 101 |
| | | LLQEQGQTQSAEDVLRQATELIK | AAELLVRLIKLLIEIAKLLQEQGQ | |
| | | RVTELLEKIAKNSDTPELALRAA | TQSAEDVLRQATELIKRVTELLEK | |
| | | ELLVRLIKLLIEIAKLLQEQGNK | IAKNSDTPELALRAAELLVRLIKL | |
| | | EEATKVLREAEELIERVFELLKK | LIEIAKLLQEQGNKEEATKVLREA | |
| | | IAENSDTPELAKRAEELIERLIE | EELIERVFELLKKIAENSDTPELA | |
| | | LLEEIAKLLEEAGRRKEALRVLL | KRAEELIERLIELLEEIAKLLEEA | |
| | | KALELLLRLLKKSLDELERSLEE | GRRKEALRVLLKALELLLRLLKKS | |
| | | LEKNPSEDALVENNRLNVKNNRI | LDELERSLEELEKNPSEDALVENN | |
| | | IVKVLEMIAKVLKMNAKAV** | RLNVKNNRIIVKVLEMIAKVLKMN | |
| | | SEQ ID NO: 37 | AKAV** | |
| | | | SEQ ID NO: 38 | |
| 95 | O3_HF_ | (M)MALEKDRRALEALRRAQEAE | (METDTLLLWVLLLWVPGSTGDGS) | 1, 2, 3, 4, 5, 6, |
| | DG_04 | KKGDVEEAVRAAQEAVRAAKESG | ALEKDRRALEALRRAQEAEKKGD | 7, 9, 10, 13, |
| | | ASWILRLVAEQALRIAKEAEKQG | VEEAVRAAQEAVRAAKESGASWIL | 16, 43, 44, 45, |
| | | NVEVAVKAARVAVEAAKQAGDND | RLVAEQALRIAKEAEKQGNVEVAV | 46, 48, 49 |
| | | VLRKVAEQALRIAKEAEKQGNVD | KAARVAVEAAKQAGDNDVLRKVAE | |
| | | VAAKAAQVAAEAAKQAGDKDMLE | QALRIAKEAEKQGNVDVAAKAAQV | |
| | | KVAKVAEQIAKAAEKEGDKKVSI | AAEAAKQAGDKDMLEKVAKVAEQI | |
| | | DATRIALEASLAALEIILEELKE | AKAAEKEGDKKVSIDATRIALEAS | |
| | | MLERLEKNPDKDVIVKVLKVIVK | LAALEIILEELKEMLERLEKNPDK | |
| | | AIEASVKNQKI SAKNQKALAELA | DVIVKVLKVIVKAIEASVKNQKIS | |
| | | (GSWSGGGSEQKLISEEDIGGS) | AKNQKALAELA(GSWSGGGSEQKL | |
| | | SEQ ID NO: 39 | ISEEDLGGS) | |
| | | | SEQ ID NO: 40 | |
| 96 | O3_HF_ | (MHHHHHHGGSEQKLISEEDLSG | (METDTLLLWVLLLWVPGSTGDGS | 1, 3, 4, 5, 7, 8, |
| | DG_05 | GGSWSGS)GRELKQLAEVLEEIQ | HHHHHHGGSEQKLISEEDLSGGGS | 9, 11, 12, 15, |
| | | RLAEEARKLMTDEEEAKKIQEEA | WSGS)GRELKQLAEVLERIORLAE | 16, 39, 40, 42, |
| | | ERAKRMLASAVWAVTDNEVIEKL | EARKLMTDEEEAKKIQEEAERAKR | 43, 46, 47, |
| | | LEVVKEIIRLAEEAMKKMTDEEE | MLASAVWAVTDNEVIEKLLEVVKE | 49, 50, 51, 237, |
| | | AAKIAKEALEAIKMLARAVEEVT | IIRLAEEAMKKMTDEEEAAKIAKE | 240, 241, 243, |
| | | DNEVIEKLLEVVKEIIRAAEEAM | ALEAIKMLARAVEEVTDNEVIEKL | 245, 246, |
| | | KLMRNEEEAAKIAKKALEATKAL | LEVVKEIIRAAEEAMKLMRNEEEA | 249 |
| | | AEAVEAIKNKEEIERLLTLVKEL | AKIAKKALEAIKALAEAVEAIKNK | |
| | | IRKAEEEARRMSDREKAAEIIER | EEIERLLTLVKELIRKAEEEARRM | |
| | | ALEKIKKLAKLAKALADLEKALR | SDREKAAEIIERALEKIKKLAKLA | |
| | | ELKKSLDELERSLEELERNPSEA | KALADLEKALRELKKSLDELERSL | |
| | | ALVENNRINVENNKIIVEVLRII | EELERNPSEAALVENNRLNVENNK | |
| | | AEVLKINAELA** | IIVEVLRIIAEVLKINAELA** | |
| | | SEQ ID NO: 41 | SEQ ID NO: 42 | |
| 99 | O3_HF_ | (MHHHHHHGGSEQKLISEEDLSG | (METDTLLLWVLLLWVPGSTGDGS | 23, 24, 26, 27, |
| | DG_08 | GGSWSGS)TKEDARSTCEKAARK | HHHHHHGGSEQKLISEEDLSGGGS | 30, 31, 33, 34, |
| | | AAESNDEEVAKRAAIECARVAME | WSGS)TKEDARSTCEKAARKAAES | 35, 37, 38, |
| | | AGMPTKEAARSFCEAAARAAAES | NDEEVAKRAAIECARVAMEAGMPT | 43, 76, 79, 80 |
| | | NDEEVAKIAAKACLIVARAAGMP | KEAARSFCEAAARAAAESNDEEVA | |
| | | TEEAARSFCEAAAKAAAEAGDAR | KIAAKACLIVARAAGMPTEEAARS | |
| | | VAKIAEKACREVARQAGMPEKDA | FCEAAAKAAAEAGDARVAKIAEKA | |
| | | DRAFKEAMKQAIEETLKRLEDSL | CREVARQAGMPEKDADRAFKEAMK | |
| | | RELRRILEELKEMLERLEKNPDK | QAIEETLKRLEDSLRELRRILEEL | |
| | | DVIVKVLKVIVKAIEASVENQRI | KEMLERLEKNPDKDVIVKVLKVIV | |
| | | SAKNQAALAALA** | KAIEASVENQRISAKNQAALAALA | |
| | | SEQ ID NO: 43 | ** | |
| | | | SEQ ID NO: 44 | |

In another embodiment, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:45-58, wherein residues in parentheses are optional (see Table 3), and may be present or absent; wherein any N-terminal methionine residues are optional and may be present or absent; wherein some or all of the optional residues may be absent and not included for determining percent identity.

The isolated polypeptides of this embodiment form trimers that can be used to trimerize molecules (such as protein antigens) fused to them. The reference sequences are shown in Table 3, and include bacterially-expressed and mammalian-expressed versions.

TABLE 3

| Name | Bacterially-expressed | Mammalian-expressed |
|---|---|---|
| I3_HF_DG_02 | (MHHHHHHGGSEQKLISEEDLSGG | (METDTLLLWVLLLWVPGSTGDGSH |
| | GSWSGS)PRLLLEEAKERVETIER | HHHHHGGSEQKLISEEDLSGGGSWS |
| | LIFTAKALQAKGNKEEAEKVLREA | GS)PRLLLEEAKERVETIERLIFTA |
| | REQIREVTLILELIAKDSDTPELA | KALQAKGNKEEAEKVLREAREQIRE |
| | TRAAELLVRLIKLLIEIAKLLQEQ | VTLILELIAKDSDTPELATRAAELL |
| | GNKEEAEKVLREATELIKRVTELL | VRLIKLLIEIAKLLQEQGNKEEAEK |
| | EKIAKNSDTPELALRAAELLVRLI | VEREATELIKRVTELLEKIAKNSDT |
| | KLLIEIAKLLQEQGNKEEAEKVER | PELALRAAELLVRLIKLLIEIAKLL |
| | EAEELIKRVLELLEKIAENADTEE | QEQGNKEEAEKVLREAEELIKRVLE |
| | LARRAAELIKRLIELLKEIAKLLE | LLEKIAENADTEELARRAAELIKRL |
| | EAGKKDEAEKVKEKAKEMKERVDI | IELLKEIAKLLEEAGKKDEAEKVKE |
| | LETLIELERSLRELRRILEELKEM | KAKEMKERVDILETLIELERSLREL |
| | LERLERNPDKDVIVKVLKVIVKAI | RRILEELKEMLERLERNPDKDVIVK |
| | EASVENQRISAENQKALARLA** | VLKVIVKAIEASVENQRISAENQKA |
| | SEQ ID NO: 45 | LARLA** |
| | | SEQ ID NO: 46 |
| I3_HF_DG_09 | MSYDERARKAVKRYVKEEGGSEEE | (METDTLLLWVLLLWVPGSTGDGS)SY |
| | AEREAEKVREEIRKKASDKYLIQA | DERARKAVKRYVKEEGGSEEEAEREAE |
| | AAAVVAYVIELGGSPDEAVKLAEA | KVREEIRKKASDKYLIQAAAAVVAYVI |
| | VVRAIKAAADDSYLEQAAAAVVAF | ELGGSPDEAVKLAEAVVRAIKAAADDS |
| | VIRKGGSPMEAVIKAKEVVDRIKE | YLEQAAAAVVAFVIRKGGSPMEAVIKA |
| | AADSREATRKAARMVATVIQAGGS | KEVVDRIKEAADSREATRKAARMVATV |
| | PEEAVKDAKKLVDLLRALRELEKA | IQAGGSPEEAVKDAKKLVDLLRALREL |
| | LRELKKSLDELERSLEELEKNPSE | EKALRELKKSLDELERSLEELEKNPSE |
| | DALVENNRLNVENNKIIVEVLRII | DALVENNRLNVENNKIIVEVLRIIAEV |
| | AEVLKINAELV(GSWSGGGSEQKL | LKINAELV(GSWSGGGSEQKLISEEDL |
| | ISEEDLGGS) | GGS) |
| | SEQ ID NO: 47 | SEQ ID NO: 48 |
| O3_HF_DG_01 | (MHHHHHHGGSEQKLISEEDLSGG | (METDTLLLWVLLLWVPGSTGDGSHHHH |
| | GSWSGS)PELEEWIRRAKEVAKEV | HHGGSEQKLISEEDLSGGGSWSGS)PEL |
| | EKVAQRAEEEGNPDLRDSAKELRK | EEWIRRAKEVAKEVEKVAQRAEEEGNPD |
| | AVELAILIAKMLGNPELVEWVARA | LRDSAKELRKAVELAILIAKMLGNPELV |
| | AKVAAEVIKVAIQAEKEGNRDLER | EWVARAAKVAAEVIKVAIQAEKEGNRDL |
| | AALELVRAVIEAINIAVVLGDPRL | FRAALELVRAVIEAINIAVVLGDPRLVE |
| | VEAVARAAKVAAEVIKVAILAEKM | AVARAAKVAAEVIKVAILAEKMGAREMF |
| | GAREMFRKALELVRKVIEAIETAV | RKALELVRKVIEAIETAVIEGDPEKVER |
| | IEGDPEKVERVAREATKEALKILL | VAREATKEALKILLWLLEKLLRELKKSL |
| | WLLEKLLRELKKSLDELERSLEEL | DELERSLEELEKNPSEDALVENNRINVE |
| | EKNPSEDALVENNRLNVENNKIIV | NNKIIVKVLEMIARVLKMNAKAV** |
| | KVLEMIARVLKMNAKAV** | SEQ ID NO: 50 |
| | SEQ ID NO: 49 | |
| O3_HF_DG_02 | (MHHHHHHGGSEQKLISEEDLSGG | (METDTLLLWVLLLWVPGSTGDGSHHHH |
| | GSWSGS)PELEEWIRRAKEVAKEV | HHGGSEQKLISEEDLSGGGSWSGS)PEL |
| | EKVAQRAEEEGNPDLRDSAKELRK | EEWIRRAKEVAKEVEKVAQRAEEEGNPD |
| | AVELAIEIARWLGNPELVEWVARA | LRDSAKELRKAVELAIEIARWLGNPELV |
| | AKVAAEVIKVAIQAEKEGNRDLER | EWVARAAKVAAEVIKVAIQAEKEGNRDL |
| | AALELVRAVIEAINIAVVLGDPRL | FRAALELVRAVIEAINIAVVLGDPRLVE |
| | VEAVARAAKVAAEVIKVAIDAEKA | AVARAAKVAAEVIKVAIDAEKAGAREME |
| | GAREMFRRALELVREVIEAIEEAV | RRALELVREVIEAIEEAVIEGDPERVER |
| | IEGDPERVERVARKATKEALDIAL | VARKATKEALDIALKLLEMLLQRLREML |
| | KLLEMLLQRLREMLRKLKESLEEL | RKLKESLEELKKNPSEDALVRNNELIVE |
| | KKNPSEDALVRNNELIVEVLRVIV | VLRVIVEVLSMIAKVLKLNAKLV** |
| | EVLSMIAKVLKLNAKLV** | SEQ ID NO: 52 |
| | SEQ ID NO: 51 | |
| O3_HF_DG_08 | (MHHHHHHGGSEQKLISEEDLSGG | (METDTLLLWVLLLWVPGSTGDGSHHHH |
| | GSWSGS)TKEDARSTCEKAARKAA | HHGGSEQKLISEEDLSGGGSWSGS)TKE |
| | ESNDEEVAKRAAIECARVAMEAGM | DARSTCEKAARKAAESNDEEVAKRAATE |
| | PTKEAARSFCEAAARAAAESNDEE | CARVAMEAGMPTKEAARSFCEAAARAAA |
| | VAKIAAKACLIVARAAGMPTEEAA | ESNDEEVAKIAAKACLIVARAAGMPTEE |
| | RSFCEAAAKAAAEAGDARVAKIAE | AARSFCEAAAKAAAEAGDARVAKIAEKA |
| | KACREVARQAGMPEKDADRAFKEA | CREVARQAGMPEKDADRAFKEAMKQAIE |
| | MKQAIEETLKRLEDSLRELRRILE | ETLKRLEDSLRELRRILEELKEMLERLE |
| | ELKEMLERLEKNPDKDVIVKVLKV | KNPDKDVIVKVLKVIVKAIEASVENQRI |
| | IVKAIEASVENQRISAKNQAALAA | SAKNQAALAALA** |
| | LA** | SEQ ID NO: 54 |
| | SEQ ID NO: 53 | |
| T3_HF_ DG_04 | (MHHHHHHGGSEQKLISEEDLSGG | (METDTLLLWVLLLWVPGSTGDGSHHHHHH |
| | GSWSGS)SEKEKVEELAQRIREQL | GGSEQKLISEEDLSGGGSWSGS)SEKEKVE |
| | PDTRLALMAQALANLANALDDSEA | ELAQRIREQLPDTRLALMAQALANLANALD |
| | LKVVYLALRIVQOLPDTELAREAL | DSEALKVVYLALRIVQQLPDTELAREALEL |
| | ELALDAAKSTDSKALEVVKLALRI | ALDAAKSTDSKALEVVKLALRIVQLLPDTE |
| | VQLLPDTEDAREALELAKEAVKST | DAREALELAKEAVKSTDEEERKKVKIKLKL |
| | DEEERKKVKIKLKLLEALAELEKA | LEALAELEKALRELKKSLDELERSLEELEK |
| | LRELKKSLDELERSLEELEKNPSE | NPSEDALVENNRLNVENNKIIVEVLRIIAE |
| | DALVENNRLNVENNKIIVEVLRII | VIKINAKLA** |
| | AEVLKINAKLA** | SEQ ID NO: 56 |
| | SEQ ID NO: 55 | |
| T3_HF_ DG_05 | (MHHHHHHGGSEQKLISEEDLSGG | (METDTLLLWVLLLWVPGSTGDGSHHHHHHG |
| | GSWSGS)PRERLEEAKERVEEIRE | GSEQKLISEEDLSGGGSWSGS)PRERLEEAK |
| | LIDKARKLQEQGNRVDATAVLMEA | ERVEEIRELIDKARKLQEQGNRVDATAVLME |
| | RSQIREVTRELEEIAKNSDTPELA | ARSQIREVTRELEEIAKNSDTPELATRAAEL |
| | TRAAELLVRLIKLLIEIAKLLQEQ | LVRLIKLLIEIAKLLQEQGQTQSAEDVLREA |
| | GQTQSAEDVLREATELIKRVTELL | TELIKRVTELLEKIAKNSDTPELALRAAELL |
| | EKIAKNSDTPELALRAAELLVRLI | VRLIKLLIEIAKLLQEQGNKEEATKVLREAE |
| | KLLIEIAKLLQEQGNKEEATKVLR | ELIERVFELLKKIAENSDTPELAKRAEELIE |
| | EAEELIERVFELLKKIAENSDTPE | RLIELLEEIAKLLEEAGRRKEALRVLLKALE |
| | LAKRAEELIERLIELLEEIAKLLE | LLLRLLKKSLDELERSLEELEKNPSEDALVE |
| | EAGRRKEALRVLLKALELLLRLLK | NNRLNVKNNRIIVKVLEMIAKVLKMNAKAV* |
| | KSLDELERSLEELEKNPSEDALVE | * |
| | NNRLNVKNNRIIVKVLEMIAKVLK | SEQ ID NO: 58 |
| | MNAKAV** | |
| | SEQ ID NO: 57 | |

In another embodiment, the disclosure provides fusion proteins, comprising:
(a) the polypeptide of any embodiment or combination of embodiments of the disclosure; and
(b) one or more additional polypeptides.

The fusion proteins of the disclosure can be used, for example, to display the one or more additional polypeptides on nanoparticles formed by the polypeptides based on SEQ ID NO: 1-44, or on trimers formed by the polypeptides based on SEQ ID NO: 45-58. Any one or more additional polypeptides may be used in the fusion proteins as suitable for an intended purpose. In various embodiments, the one or more additional polypeptides may comprise a diagnostic polypeptide, a therapeutic polypeptide, a detectable polypeptide, an antigen, etc.

The fusion protein may further comprise optional amino acid linkers between the polypeptide and the one or more additional polypeptides.

In one embodiment, the one or more additional polypeptides comprise an antigen. Any antigen may be used as appropriate for an intended purpose. In some embodiments, the antigen comprises a bacterial or viral antigen. In another embodiment, the bacterial or viral antigen comprises a coronavirus antigen, including but not limited to a SARS COV-2 antigen. In certain non-limiting embodiments, the coronavirus antigen comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 59-70.

TABLE 4
Rpk9_RBD_SARS-CoV-2
RFPNITNLCPFGEVFNATRFASVYAWNRKRISNCVADESVLYNSASFSTFKCYGVSPTKLNDLCWINIYADSF
VIRGDEVRQIAPGQTGKIADYNYKLPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPATVCGPKKST SEQ ID NO: 59
WT_RBD_SARS-CoV-2
RFPNITNLCPFGEVFNATRFASVYAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPATVCGPKKST SEQ ID NO: 60
Rpk4_RBD_SARS-CoV-2
RFPNITNLCPFGEVFNATRFASVYAWNRKRISNCVADYSVLYNSASFSTFKCYGVSPTKLNDLCWTNVYADSF
VIRGDEVRQIAPGQTGKIADYNYKLPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPATVCGPKKST SEQ ID NO: 61
WT_RBD_SARS-CoV
RFPNITNLCPFGEVFNATKFPSVYAWERKKISNCVADYSVLYNSTFFSTFKCYGVSATKLNDLCFSNVYADSF
VVKGDDVRQIAPGQTGVIADYNYKLPDDFMGCVLAWNTRNIDATSTGNYNYKYRYLRHGKLRPFERDISNVPF
SPDGKPCTPPALNCYWPLNDYGFYTTTGIGYQPYRVVVLSFELLNAPATVCGPKLST SEQ ID NO: 62
Rpk4_RBD_SARS-CoV
RFPNITNLCPFGEVFNATKFPSVYAWERKKISNCVADYSVLYNSTFFSTFKCYGVSATKLNDLCWSNVYADSF
VVKGDDVRQIAPGQTGVIADYNYKLPDDFMGCVLAWNTRNIDATSTGNYNYKYRYLRHGKLRPFERDISNVPF
SPDGKPCTPPALNCYWPLNDYGFYTTTGIGYQPYRVVVLSFELLNAPATVCGPKLST SEQ ID NO: 63
Rpk9_RBD_SARS-CoV
RFPNITNLCPFGEVFNATKFPSVYAWERKKISNCVADFSVLYNSTFFSTFKCYGVSATKLNDLCWSNIYADSF
VVKGDDVRQIAPGQTGVIADYNYKLPDDFMGCVLAWNTRNIDATSTGNYNYKYRYLRHGKLRPFERDISNVPF
SPDGKPCTPPALNCYWPLNDYGFYTTTGIGYQPYRVVVLSFELLNAPATVCGPKLST SEQ ID NO: 64
WT_RBD_BtKY72
RFPNITNLCPFGQVFNASNFPSVYAWERLRISDCVADYAVLYNSSSSFSTFKCYGVSPTKLNDLCFSSVYADY
FVVKGDDVRQIAPAQTGVIADYNYKLPDDFTGCVLAWNTNSVDSKSGNNFYYRLFRHGKIKPYERDISNVLYN
SAGGTCSSISQLGCYEPLKSYGFTPTVGVGYQPYRVVVLSFELLNAPATVCGPKKST SEQ ID NO: 65
Rpk4_RBD_BtKY72
RFPNITNLCPFGQVFNASNFPSVYAWERLRISDCVADYAVLYNSSSSFSTFKCYGVSPTKLNDLCWSSVYADY
FVVKGDDVRQIAPAQTGVIADYNYKLPDDFTGCVLAWNTNSVDSKSGNNFYYRLFRHGKIKPYERDISNVLYN
SAGGTCSSISQLGCYEPLKSYGFTPTVGVGYQPYRVVVLSFELLNAPATVCGPKKST SEQ ID NO: 66
Rpk9_RBD_BtKY72
RFPNITNLCPFGQVFNASNFPSVYAWERLRISDCVADFAVLYNSSSSFSTFKCYGVSPTKLNDLCWSSIYADY
FVVKGDDVRQIAPAQTGVIADYNYKLPDDFTGCVLAWNTNSVDSKSGNNFYYRLFRHGKIKPYERDISNVLYN
SAGGTCSSISQLGCYEPLKSYGFTPTVGVGYQPYRVVVLSFELLNAPATVCGPKKST SEQ ID NO: 67
WT_RBD_RmYN02
RFPNITNFCPFDKVFNATRFPNVYAWQRTKISDCIADYTVLYNSTSFSTFKCYGVSPSKLIDLCFTSVYADTF
LIRFSEVRQIAPGETGVIADYNYKLPDDFTGCVLAWNTAQQDIGSYFYRSHRAVKLKPFERDLSSDENGVRTL
STYDFNPNVPLDYQATRVVVLSFELLNAPATVCGPKLST SEQ ID NO: 68
Rpk4_RBD_RmYN02
RFPNITNFCPFDKVFNATRFPNVYAWQRTKISDCIADYTVLYNSTSFSTFKCYGVSPSKLIDLCWTSVYADTF
LIRFSEVRQIAPGETGVIADYNYKLPDDFTGCVLAWNTAQQDIGSYFYRSHRAVKLKPFERDLSSDENGVRTL
STYDFNPNVPLDYQATRVVVLSFELLNAPATVCGPKLST SEQ ID NO: 69
Rpk9_RBD_RmYN02
RFPNITNFCPFDKVFNATRFPNVYAWQRTKISDCIADFTVLYNSTSFSTFKCYGVSPSKLIDLCWTSIYADTF
LIRFSEVRQIAPGETGVIADYNYKLPDDFTGCVLAWNTAQQDIGSYFYRSHRAVKLKPFERDLSSDENGVRTL
STYDFNPNVPLDYQATRVVVLSFELLNAPATVCGPKLST SEQ ID NO: 70

In various further embodiments, the fusion proteins comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 72, 74, 76, 78, 80, 82, 84, 86, 88, and 90 (see Table 5), wherein residues in parentheses are optional and may be present or deleted. These fusion proteins display the Rpk9 RBD_SARS-COV-2 antigen (SEQ ID NO: 59). The name of each fusion protein listed in table 5 indicates which polypeptide forms part of the fusion protein. For example, SEQ ID NO:74 is named Rpk9_RBD_SARS-COV-2_KWOCA-18, which is a fusion between Rpk9_RBD_SARS-COV-2 (SEQ ID NO:59) and KWOCA-18, which is also named I3_HF_OG_18 (see Table 1; SEQ ID NO: 3 or 4). All of these designs were shown to retain antigenicity of the antigen. A number were tested and shown to both secrete and assemble; see the Examples for further details. For reference, an example amino acid sequence and DNA sequence to be used for nucleoside modified mRNA synthesis using, by way of non-limiting example, NI-Methylpseudouridine-5′-Triphosphate are also provided. The sequence of SEQ ID NO:74 is shown below, with optional residues highlighted and in parentheses, including a linker positioned between the two domains (i.e., signal sequence-additional polypeptide antigen-linker-polypeptide).

(MGILPSPGMPALLSLVSLLSVLLMGCVAETGT)RFPNITNLCPFGEVFN
ATRFASVYAWNRKRISNCVADFSVLYNSASFSTFKCYGVSPTKLNDLCWT
NIYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQ
SYGFQPTNGVGYQPYRVVVLSFELLHAPATVCGPKKST(GGSGGSGSGGS
GGSGS)SDEEEAREWAERALKAALEAAEQALREGDEDAFKCAVELLEQAL
EARKKKDSEEAEAVYWAARAVLAALEALEQAKREGDEDARRCAEELLRLA
CEAARKKNSEQARAVYEAARAVLAALRALEAAKRAGMEEARKEAEELLRR
ACEAARKQDPELARAVRDKAELLKALADLFKALKELKKSLDELERSLEEL
EKNPSEDALVENNRLNVENNKIIVEVLRIIAEVLRINARAV

In the DNA sequences shown below, the arrangement is (optional initiator) (optional S′UTR)-fusion protein open reading frame-(stop codons) (optional 3′UTR) (optional PolyA tail)

TABLE 5
Rpk9_RBD_SARS-CoV-2_13-01-NS
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKLNDLCWTNIYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQPYRVVV
LSFELLHAPATVCGPKKST(GGSGGSGSGGSGGSGSEKAAKAREAAR)KMEELFKEHKIVAVERANSVEEAKK
KALAVFLGGVDLIEITFTVPDADTVIKELSFLKEMGAIIGAGTVTSVEQAREAVESGAEFIVSPHLDEEISQF
AKEEGVFYMPGVMTPTELVKAMKLGHTILKLFPGEVVGPQFVEAMKGPFPNVKFVPTGGVNLDNVAEWFEAGV
QAVGVGEALNEGTPVEVAEKAKAFVEKIEGATE (SEQ ID NO: 72)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCTTTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTTCACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCGAGAAGGCCGCCAAGGCCGAGGAGGCCGCCAGGAAGATGGAGGAGCTCTTTAAGGAGCACAA
AATCGTGGCGGTGCTCAGGGCAAATAGCGTAGAAGAGGCCAAAAAGAAGGCACTCGCCGTCTTTCTCGGAGGC
GTGGACCTGATAGAGATCACATTCACGGTGCCAGATGCTGATACGGTCATTAAGGAGCTGAGTTTTCTCAAAG
AGATGGGCGCCATCATAGGCGCCGGCACCGTGACATCCGTGGAGCAGGCCAGAGAAGCCGTCGAGTCCGGAGC
CGAATTCATAGTGTCCCCTCATCTCGATGAGGAGATCAGTCAGTTTGCCAAGGAGGAGGGAGTGTTTTATATG
CCCGGAGTGATGACGCCGACGGAGCTAGTGAAGGCCATGAAACTGGGCCACACCATCCTCAAGCTGTTTCCTG
GAGAAGTGGTTGGTCCCCAATTTGTCGAAGCCATGAAGGGACCATTTCCAAACGTGAAATTCGTGCCGACCGG
AGGAGTTAACTTAGACAACGTTGCCGAGTGGTTTGAGGCAGGCGTTCAGGCGGTCGGGGTCGGAGAGGCCCTC
AACGAAGGAACACCAGTGGAAGTCGCCGAGAAGGCCAAAGCATTTGTGGAGAAAATCGAGGGAGCCACTGAA
(TGATGA)(CTCGAGCTGGTACTGCATGCACGCAATGCTAGCTGCCCCTTTCCCGTCCTGGGTACCCCGAGTCT
CCCCCGACCTCGGGTCCCAGGTATGCTCCCACCTCCACCTGCCCCACTCACCACCTCTGCTAGTTCCAGACAC
CTCCCAAGCACGCAGCAATGCAGCTCAAAACGCTTAGCCTAGCCACACCCCCACGGGAAACAGCAGTGATTAA
CCTTTAGCAATAAACGAAAGTTTAACTAAGCTATACTAACCCCAGGGTTGGTCAATTTCGTGCCAGCCACACC
CTGGAGCTAGC){AAAAAAAAAAAAAAAAAAAAAAAAAAAAAAGCATATGACTAAAAAAAAAAAAAAAAAAAA
AAAAAAAAAAAAAAAAAAAAAAAAAAAAAAAAAAAAAAAAAAAAAAAAAA) (SEQ ID NO: 73)
Rpk9_RBD_SARS-CoV-2_KWOCA-18
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKLNDLCWTNIYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQPYRVVV
LSFELLHAPATVCGPKKST(GGSGGSGSGGSGGSGS)SDEEEAREWAERALKAALEAAEQALREGDEDAFKCA
VELLEQALEARKKKDSEEAEAVYWAARAVLAALEALEQAKREGDEDARRCAEELLRLACEAARKKNSEQARAV
YEAARAVLAALRALEAAKRAGMEEARKEAEELLRRACEAARKQDPELARAVRDKAELLKALADLFKALKELKK
SLDELERSLEELEKNPSEDALVENNRLNVENNKIIVEVERITAEVERINARAV(SEQ ID NO: 74)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCTTTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTTCACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCAGTGATGAGGAGGAAGCTCGCGAATGGGCTGAACGGGCACTCAAAGCCGCTCTGGAGGCCGC
AGAACAAGCACTGCGCGAAGGAGACGAGGACGCTTTTAAATGCGCTGTTGAACTTCTGGAACAGGCCCTCGAA
GCTCGGAAGAAAAAGGATAGCGAGGAAGCTGAAGCCGTCTATTGGGCAGCTAGAGCTGTCCTCGCCGCGCTGG
AAGCTCTGGAACAAGCAAAACGCGAGGGAGATGAGGATGCTAGACGCTGTGCCGAGGAATTGCTGAGGCTGGC
CTGCGAGGCCGCGAGAAAGAAGAACAGCGAACAGGCTAGGGCCGTCTATGAAGCAGCTAGGGCTGTTTTGGCC
GCACTGAGGGCTCTGGAAGCCGCAAAACGCGCCGGAATGGAGGAAGCACGCAAGGAAGCCGAGGAGTTGCTCC
GAAGAGCATGCGAAGCCGCCCGCAAGCAGGATCCTGAGCTCGCTAGAGCAGTTCGGGATAAAGCTGAACTCCT
CAAAGCTCTCGCCGACTTGTTCAAGGCCCTCAAGGAACTTAAGAAGTCCCTTGACGAGCTCGAGCGGTCCCTC
GAAGAGCTGGAGAAGAACCCCAGCGAGGATGCTCTGGTCGAGAATAATCGGCTTAACGTGGAGAACAATAAGA
TTATCGTCGAAGTGCTCCGCATTATCGCTGAAGTGCTCCGAATTAATGCACGCGCCGTT(TGATGA)(CTCGA
GCTGGTACTGCATGCACGCAATGCTAGCTGCCCCTTTCCCGTCCTGGGTACCCCGAGTCTCCCCCGACCTCGG
GTCCCAGGTATGCTCCCACCTCCACCTGCCCCACTCACCACCTCTGCTAGTTCCAGACACCTCCCAAGCACGC
AGCAATGCAGCTCAAAACGCTTAGCCTAGCCACACCCCCACGGGAAACAGCAGTGATTAACCTTTAGCAATAA
ACGAAAGTTTAACTAAGCTATACTAACCCCAGGGTTGGTCAATTTCGTGCCAGCCACACCCTGGAGCTAGC)
(AAAAAAAAAAAAAAAAAAAAAAAAAAAAAAGCATATGACTAAAAAAAAAAAAAAAAAAAAAAAAAAAAAAAAA
AAAAAAAAAAAAAAAAAAAAAAAAAAAAAAAAAAAAA)(SEQ ID NO: 75)
Rpk9_RBD_SARS-CoV-2_KWOCA-4
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKLNDLCWTNIYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPINGVGYQPYRVVV
LSFELLHAPATVCGPKKST(GGSGGSGSGGSGGSGS)TEVEKKAREVAKEAVELASLLRSETAIRVAQAILEA
AEAAKRAAEQGKTEVAKLALKVLEEAIELAKEKRSEEALKVVLEIARAALAAAQAAEEGFTDVAKMALEVLER
AIELAKDDRSEEALKEVLEIARAALAAAQLAKKGRDDEARKILMKLRIRITLRKLEESLRELRRILEELKEML
ERLEKNPDKDVIVKVLKVIVKAIEASVENQRISAENQKALAELA (SEQ ID NO: 76)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCTTTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTTCACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCACTGAAGTCGAGAAGAAGGCCAGAGAGGTCGCTAAGGAAGCGGTTGAGCTGGCATCTCTTCT
GCGTAGCGAAACAGCAATTCGAGTAGCCCAAGCTATCCTTGAAGCCGCCGAGGCAGCCAAACGGGCAGCTGAA
CAGGGGAAGACCGAAGTTGCAAAACTCGCCCTGAAGGTACTCGAGGAAGCCATTGAACTGGCTAAAGAAAAAC
GGAGCGAGGAAGCCCTCAAAGTTGTTCTGGAAATCGCCAGGGCTGCACTGGCCGCCGCACAAGCAGCTGAGGA
AGGGTTCACTGATGTCGCTAAAATGGCCCTCGAGGTCCTGGAAAGAGCAATAGAGCTGGCAAAGGATGACCGC
TCCGAAGAGGCTCTGAAGGAAGTTCTGGAGATCGCCAGAGCAGCCCTCGCTGCGGCGCAGCTGGCCAAAAAAG
GCCGCGATGATGAAGCTCGGAAAATTCTGATGAAACTGAGGATTCGCATCACACTGCGGAAATTGGAGGAGTC
ACTTCGCGAGTTGCGGAGGATCCTTGAGGAGCTCAAAGAAATGCTTGAGCGCTTGGAGAAAAATCCTGACAAA
GACGTCATTGTAAAGGTCCTCAAAGTGATTGTCAAGGCTATTGAGGCCTCCGTGGAGAATCAACGCATTTCCG
CTGAGAATCAGAAGGCCCTTGCTGAATTGGCA(TGATGA)(CTCGAGCTGGTACTGCATGCACGCAATGCTAG
CTGCCCCTTTCCCGTCCTGGGTACCCCGAGTCTCCCCCGACCTCGGGTCCCAGGTATGCTCCCACCTCCACCT
GCCCCACTCACCACCTCTGCTAGTTCCAGACACCTCCCAAGCACGCAGCAATGCAGCTCAAAACGCTTAGCCT
AGCCACACCCCCACGGGAAACAGCAGTGATTAACCTTTAGCAATAAACGAAAGTTTAACTAAGCTATACTAAC
CCCAGGGTTGGTCAATTTCGTGCCAGCCACACCCTGGAGCTAGC)(AAAAAAAAAAAAAAAAAAAAAAAAAAA
AAAGCATATGACTAAAAAAAAAAAAAAAAAAAAAAAAAAAAAAAAAAAAAAAAAAAAAAAAAAAAAAAAAAAA
AAAAAAAAAA) (SEQ ID NO: 77)
Rpk9_RBD_SARS-CoV-2_KWOCA-46
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKLNDLCWTNIYADSFVIRGDEVRQIAPGQTGKIADYNYKLPDDFTGCVIAWNSN
NLDSKVGGNYNYLYRLFRKSNLKPFERDISTEIYOAGSTPCNGVEGFNCYFPLQSYGFQPTNGVGYQPYRVVV
LSFELLHAPATVCGPKKST(GGSGGSGSGGSGGSGS)TEEKIEEARQAIKEAERALREGDPREAEMAVRIALA
LVRMLERLARKTGSTEVLIEAARLAIEVARVALKVGSPETAREAVRTALELVQELERQARKTGSTEVLIEAAR
LAIEVARVAFKVGSPETAKEAVRTALELVKELIQQALKTGSDEVLERAAELAKEVARVAKEVGDPRAARKADM
VAKIADTLRELMESLRELRRILEELKEMLERLEKNPDKDVIVKVLKVIVKAIEASVENQRISAENQAALASLA
SEQ ID NO: 78)
DNA
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCTTTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTTCACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCACCGAGGAGAAAATCGAAGAGGCACGGCAGGCAATCAAGGAGGCTGAACGGGCACTGCGCGA
AGGAGACCCTCGATTTGCTGAAATGGCTGTCAGAATCGCCCTTGCCTTGGTTCGGATGCTGGAACGCCTCGCC
CGCAAGACCGGATCCACTGAGGTTCTCATTGAGGCAGCAAGGCTTGCTATTGAGGTGGCTCGGGTGGCCCTCA
AGGTCGGTTCGCCGGAGACCGCTAGAGAAGCAGTCAGAACAGCCCTTGAACTGGTCCAAGAGTTGGAGCGGCA
AGCACGCAAGACCGGGTCTACTGAGGTGCTGATTGAAGCTGCCAGGCTCGCTATTGAGGTCGCTAGGGTCGCT
TTCAAAGTTGGTTCTCCTGAGACCGCTAAGGAAGCTGTTAGAACAGCTCTGGAACTGGTTAAGGAACTGATCC
AACAAGCGCTTAAGACCGGCTCTGACGAAGTTCTGGAGCGGGCTGCTGAACTTGCTAAGGAAGTTGCCAGGGT
GGCCAAAGAGGTTGGCGATCCTCGGGCTGCCCGCAAGGCAGATATGGTGGCCAAGATCGCCGATACACTCCGC
GAGCTTATGGAATCCCTGCGCGAGCTCAGGCGGATCCTTGAGGAGCTCAAGGAAATGCTGGAGAGACTCGAGA
AGAATCCCGATAAAGACGTGATCGTCAAAGTGCTGAAAGTGATCGTTAAGGCTATTGAAGCAAGCGTTGAGAA
CCAGCGCATTTCTGCTGAGAATCAAGCAGCTCTTGCTAGTCTGGCT(TGATGA)(CTCGAGCTGGTACTGCAT
GCACGCAATGCTAGCTGCCCCTTTCCCGTCCTGGGTACCCCGAGTCTCCCCCGACCTCGGGTCCCAGGTATGC
TCCCACCTCCACCTGCCCCACTCACCACCTCTGCTAGTTCCAGACACCTCCCAAGCACGCAGCAATGCAGCTC
AAAACGCTTAGCCTAGCCACACCCCCACGGGAAACAGCAGTGATTAACCTTTAGCAATAAACGAAAGTTTAAC
TAAGCTATACTAACCCCAGGGTTGGTCAATTTCGTGCCAGCCACACCCTGGAGCTAGC)(AAAAAAAAAAAAA
AAAAAAAAAAAAAAAAAGCATATGACTAAAAAAAAAAAAAAAAAAAAAAAAAAAAAAAAAAAAAAAAAAAAAA
AAAAAAAAAAAAAAAAAAAAAAAA) (SEQ ID NO: 79)
KWOCA-47_Rpk9_RBD_SARS-CoV-2
Amino acid:
(MGILPSPGMPALLSLVSLLSVLLMGCVAETGT)
ADELRAVAELQRENIELARKLLEAVARLQELNIDLVRKTSELTDEKTIREEIRKVKEESKRIVEEAEEEIRRA
KEDSKRIVTEALRRAREQIREKWEELEERAKRAETPEEALRAAEEMVKLIEELIRIAEMLQRAGLKEEAEDVL
REATELIKRATELLEKIAKNSDTPELALRAAELLVRLIKLLIEIAKLLQEQGNKEEAEKVLREATELIKRVAR
LLLAIALLADTPELAKRAAELLKRLIELLKEIAKLLEEEGNEDEAEKVKEEAKELEELVRWLEEQIRG(GGSG
GSGSGGSGGSGS)RFPNITNLCPFGEVFNATRFASVYAWNRKRISNCVADFSVLYNSASFSTFKCYGVSPTKL
NDLCWTNIYADSFVIRGDEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELLHAPATVCGPKKST
(SEQ ID NO: 80)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAGCCGATGAGCTACGCGCTGTAGCTGAGCTGCAGCGACTTAACATTGAACTGGCACGCAAACTTCT
CGAGGCCGTGGCCAGGCTGCAGGAGCTCAACATCGATCTGGTCCGCAAGACAAGCGAATTGACAGACGAGAAG
ACCATACGCGAGGAGATCAGAAAAGTCAAGGAGGAATCTAAACGCATTGTGGAAGAGGCAGAGGAAGAGATTA
GGCGCGCAAAGGAGGACAGCAAAAGAATCGTGACCGAGGCACTCAGGCGGGCCAGGGAACAGATAAGAGAGAA
ATGGGAAGAGCTTGAGGAGCGGGCAAAGCGAGCCGAAACACCCGAAGAGGCATTACGAGCGGCCGAGGAGATG
GTTAAGCTTATTGAAGAGCTGATTCGAATCGCAGAAATGTTGCAAAGGGGGGGGCTCAAGGAGGAGGCCGAAG
ACGTCCTCCGCGAGGCCACAGAACTCATCAAACGGGCCACCGAACTTCTCGAGAAAATCGCTAAGAACAGCGA
TACACCGGAACTGGCACTTCGGGCCGCCGAACTTTTGGTTCGTTTAATCAAGCTTTTGATCGAAATCGCAAAA
CTTCTCCAAGAACAGGGCAACAAGGAGGAGGCAGAGAAAGTCCTTCGCGAGGCAACCGAACTTATCAAAAGGG
TCGCCAGACTGCTCCTGGCCATCGCACTGCTGGCGGATACGCCAGAACTTGCAAAGCGCGCCGCCGAGCTGCT
GAAACGATTGATTGAATTGCTCAAGGAGATCGCCAAACTCCTGGAGGAGGAAGGTAACGAGGATGAGGCAGAG
AAAGTGAAGGAAGAGGCCAAGGAACTCGAGGAGCTGGTCCGTTGGCTGGAGGAACAAATCCGAGGTGGAGGCT
CTGGAGGCAGCGGCTCCGGAGGCTCTGGAGGCAGCGGCTCCAGGTTCCCTAACATCACCAATCTGTGCCCATT
CGGCGAGGTGTTTAACGCCACACGCTTTGCCTCCGTGTATGCCTGGAACCGGAAGAGAATCTCTAATTGCGTG
GCCGACTTCAGCGTGCTGTACAATAGCGCCTCCTTCTCTACCTTTAAGTGCTATGGCGTGAGCCCCACCAAGC
TGAACGATCTGTGCTGGACAAACATTTACGCCGACTCCTTTGTGATCCGGGGCGATGAAGTGAGACAGATCGC
ACCAGGACAGACCGGAAAGATCGCAGACTACAACTATAAGCTGCCTGACGATTTCACAGGCTGCGTGATCGCC
TGGAATAGCAACAATCTGGATTCCAAAGTGGGCGGCAACTACAATTATCTGTACAGGCTGTTCCGCAAGTCCA
ACCTGAAGCCATTTGAGCGGGACATCTCCACCGAGATCTACCAGGCCGGCTCTACACCCTGCAACGGCGTGGA
GGGCTTCAATTGTTATTTTCCCCTGCAGTCTTACGGCTTCCAGCCTACAAATGGCGTGGGCTATCAGCCATAC
CGGGTGGTGGTGCTGTCTTTTGAGCTGCTGCACGCACCAGCAACCGTGTGCGGCCCCAAGAAGAGCACA(TGA
TGA)(CTCGAGCTGGTACTGCATGCACGCAATGCTAGCTGCCCCTTTCCCGTCCTGGGTACCCCGAGTCTCCC
CCGACCTCGGGTCCCAGGTATGCTCCCACCTCCACCTGCCCCACTCACCACCTCTGCTAGTTCCAGACACCTC
CCAAGCACGCAGCAATGCAGCTCAAAACGCTTAGCCTAGCCACACCCCCACGGGAAACAGCAGTGATTAACCT
TTAGCAATAAACGAAAGTTTAACTAAGCTATACTAACCCCAGGGTTGGTCAATTTCGTGCCAGCCACACCCTG
GAGCTAGC)(AAAAAAAAAAAAAAAAAAAAAAAAAAAAAAGCATATGACTAAAAAAAAAAAAAAAAAAAAAAA
AAAAAAAAAAAAAAAAAAAAAAAAAAAAAAAAAAAAAAAAAAAAAAA) (SEQ ID NO: 81)
Rpk9_RBD_SARS-CoV-2_KWOCA-51
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKINDLCWTNIYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQPYRVVV
LSFELLHAPATVCGPKKST(GGSGGSGSGGSGGSGS)SDEEEAREWAERAEEAAKEALEQAKREGDEIARLCA
KMLEILAEEARRKKDSEEAEAVYWAARAVLAALEALEQAKREGDEDARRCAEELLRLACSAAARQDSEQARAV
YEAARAVLAALRALEAAKRAGMEEARKEAEELLRRACEAARKQDPELARAVRDKAELLKALADLFKALKELKK
SLDELERSLEELEKNPSEDALVENNRLNVENNKIIVEVLRIIAEVERINARAV (SEQ ID NO: 82)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCTTTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTTCACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCAGCGACGAGGAGGAAGCTCGCGAATGGGCCGAGCGCGCTGAGGAAGCTGCAAAGGAGGCTCT
CGAGCAAGCAAAGAGGGAAGGCGACGAAATCGCACGGTTATGCGCTAAAATGCTGGAAATCCTGGCCGAAGAG
GCCCGCCGCAAAAAGGATTCTGAGGAGGCTGAAGCTGTCTATTGGGCCGCTAGAGCTGTTCTGGCGGCATTGG
AGGCACTGGAGCAGGCTAAACGCGAAGGCGACGAGGATGCTAGGCGCTGTGCTGAGGAACTCCTGAGACTTGC
CTGTTCTGCCGCCGCTCGCCAAGATTCAGAGCAAGCCCGGGCTGTCTACGAGGCAGCTCGAGCTGTTCTGGCC
GCCCTGAGAGCTCTCGAAGCAGCAAAACGGGCAGGCATGGAGGAGGCTCGCAAGGAAGCTGAGGAGCTTCTTC
GCCGAGCATGCGAAGCCGCCCGCAAACAGGATCCCGAGCTGGCAAGAGCCGTCAGAGATAAGGCCGAGTTGCT
TAAGGCACTTGCTGACCTATTTAAAGCCCTGAAGGAGCTCAAGAAGTCCCTTGACGAACTGGAAAGGAGCCTC
GAAGAGCTCGAGAAGAATCCTTCCGAGGATGCACTGGTTGAGAACAATAGATTGAACGTGGAAAACAATAAGA
TTATCGTTGAGGTGCTTAGGATTATCGCCGAGGTGCTCAGAATTAATGCTAGAGCCGTC(TGATGA)(CTCGA
GCTGGTACTGCATGCACGCAATGCTAGCTGCCCCTTTCCCGTCCTGGGTACCCCGAGTCTCCCCCGACCTCGG
GTCCCAGGTATGCTCCCACCTCCACCTGCCCCACTCACCACCTCTGCTAGTTCCAGACACCTCCCAAGCACGC
AGCAATGCAGCTCAAAACGCTTAGCCTAGCCACACCCCCACGGGAAACAGCAGTGATTAACCTTTAGCAATAA
ACGAAAGTTTAACTAAGCTATACTAACCCCAGGGTTGGTCAATTTCGTGCCAGCCACACCCTGGAGCTAGC)
{AAAAAAAAAAAAAAAAAAAAAAAAAAAAAAGCATATGACTAAAAAAAAAAAAAAAAAAAAAAAAAAAAAAAAA
AAAAAAAAAAAAAAAAAAAAAAAAAAAAAAAAAAAAA) (SEQ ID NO: 83)
KWOCA-70_Rpk9_RBD_SARS-CoV-2
Amino acid:
(MGILPSPGMPALLSLVSLLSVLLMGCVAETGT)DESVDLAVKLAEALRKEAEELIKKARKTGDPELLRKALE
ALEKAVKLVEDAIKRNPDNDEAVETAVRLARELKKVAEELQERAKKTGDPELLKLALRALEVAVRAVELAIKS
NPDNDEAVKTAVELAKELEKVARELLERARKTGDDELLKLAKRALEVARRAVELALKSRPDAEEARRVYIRLT
EMELEISLTELRKILEELKEMLERLEKNPDKDVIVKVLKVIVKAIEASVENQRISAENQKALAELA(GGSGGS
GSGGSGGSGS)RFPNITNLCPFGEVFNATRFASVYAWNRKRISNCVADFSVLYNSASFSTFKCYGVSPTKLND
LCWTNIYADSFVIRGDEVRQIAPGQTGKIADYNYKLPDDFTGCVIAWNSNNLDSKVGGNYNYLYRLFRKSNLK
PFERDISTEIYQAGSTPCNGVEGFNCYFPLQSYGFQPTNGVGYQPYRVVVLSFELLHAPATVCGPKKST (SEQ
ID NO: 84)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAGATGAGTCTGTTGACCTCGCTGTAAAGCTAGCCGAAGCCCTCCGCAAGGAGGCCGAGGAGCTGAT
AAAGAAGGCTCGGAAGACAGGCGACCCCGAACTGCTTAGGAAGGCTCTCGAGGCCCTCGAGAAAGCTGTGAAG
CTTGTCGAGGACGCCATCAAACGGAATCCCGACAATGACGAGGCCGTGGAAACTGCAGTTAGGCTCGCTCGGG
AATTGAAGAAGGTCGCCGAGGAGCTGCAGGAACGGGCCAAAAAGACTGGTGACCCCGAGCTCCTCAAACTCGC
TCTGAGAGCCCTGGAGGTGGCAGTCCGCGCCGTAGAACTGGCCATCAAGTCTAATCCTGATAACGATGAGGCA
GTTAAGACTGCTGTGGAGCTTGCAAAGGAGCTCGAGAAAGTGGCACGCGAACTGTTGGAGAGAGCTCGCAAGA
CTGGGGATGACGAACTCCTGAAGCTGGCCAAGCGCGCTCTTGAAGTGGCAAGGCGCGCCGTGGAACTTGCCCT
TAAGAGCCGGCCCGATGCGGAGGAGGCAAGACGGGTCTACATTCGCCTGACCGAGATGGAGCTTGAGATTTCC
CTGACCGAACTCCGCAAAATTCTCGAGGAGCTCAAGGAAATGTTGGAACGGCTTGAGAAGAACCCAGATAAGG
ACGTCATCGTTAAGGTGCTCAAGGTTATTGTTAAAGCTATTGAAGCATCCGTCGAAAACCAGAGAATCTCAGC
CGAGAACCAGAAAGCCCTTGCTGAACTGGCAGGAGGCTCTGGAGGCAGCGGCTCCGGAGGCTCTGGAGGCAGC
GGCTCCAGGTTCCCTAACATCACCAATCTGTGCCCATTCGGCGAGGTGTTTAACGCCACACGCTTTGCCTCCG
TGTATGCCTGGAACCGGAAGAGAATCTCTAATTGCGTGGCCGACTTCAGCGTGCTGTACAATAGCGCCTCCTT
CTCTACCTTTAAGTGCTATGGCGTGAGCCCCACCAAGCTGAACGATCTGTGCTGGACAAACATTTACGCCGAC
TCCTTTGTGATCCGGGGCGATGAAGTGAGACAGATCGCACCAGGACAGACCGGAAAGATCGCAGACTACAACT
ATAAGCTGCCTGACGATTTCACAGGCTGCGTGATCGCCTGGAATAGCAACAATCTGGATTCCAAAGTGGGGGG
CAACTACAATTATCTGTACAGGCTGTTCCGCAAGTCCAACCTGAAGCCATTTGAGCGGGACATCTCCACCGAG
ATCTACCAGGCCGGCTCTACACCCTGCAACGGCGTGGAGGGCTTCAATTGTTATTTTCCCCTGCAGTCTTACG
GCTTCCAGCCTACAAATGGCGTGGGCTATCAGCCATACCGGGTGGTGGTGCTGTCTTTTGAGCTGCTGCACGC
ACCAGCAACCGTGTGCGGCCCCAAGAAGAGCACA(TGATGA)(CTCGAGCTGGTACTGCATGCACGCAATGCT
AGCTGCCCCTTTCCCGTCCTGGGTACCCCGAGTCTCCCCCGACCTCGGGTCCCAGGTATGCTCCCACCTCCAC
CTGCCCCACTCACCACCTCTGCTAGTTCCAGACACCTCCCAAGCACGCAGCAATGCAGCTCAAAACGCTTAGC
CTAGCCACACCCCCACGGGAAACAGCAGTGATTAACCTTTAGCAATAAACGAAAGTTTAACTAAGCTATACTA
ACCCCAGGGTTGGTCAATTTCGTGCCAGCCACACCCTGGAGCTAGC)(AAAAAAAAAAAAAAAAAAAAAAAAA
AAAAAGCATATGACTAAAAAAAAAAAAAAAAAAAAAAAAAAAAAAAAAAAAAAAAAAAAAAAAAAAAAAAAAA
AAAAAAAAAAAA) (SEQ ID NO: 85)
Rpk9_RBD_SARS-CoV-2_KWOCA-100
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKLNDLCWTNIYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQPYRVVV
LSFELLHAPATVCGPKKST(GGSGGSGSGGSGGSGS)TEETIEMARQLIKEAERALREGDPEEARMAVEMALA
AVRILERQARKTGSTEVLIEAARLAIEVARVALKVGSPETAREAVRTALELVQELERQARKTGSTEVLIEAAR
LAIEVARVAFKVGSPETAKEAVRTALELVKELIQQALKTGSDEVLERAAELAKEVARVAKEVGDPRAARKADM
VAKIADTLRELMESLRELRRILEELKEMLERLEKNPDKDVIVKVLKVIVKAIEASVENQRISAENQAALASLA
(SEQ ID NO: 86)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCTTTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTTCACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCACTGAGGAGACAATCGAAATGGCTAGACAACTCATAAAGGAAGCTGAGCGCGCTCTGAGAGA
AGGGGACCCCGAGGAGGCCAGAATGGCCGTGGAAATGGCTTTGGCCGCTGTCCGTATACTCGAGCGCCAAGCT
AGGAAGACCGGTTCCACCGAAGTCCTGATTGAGGCTGCTAGGCTGGCAATCGAGGTTGCCAGGGTGGCACTGA
AAGTCGGCAGCCCCGAGACAGCCAGAGAAGCTGTCAGGACAGCACTTGAACTTGTCCAAGAGTTGGAACGCCA
GGCCAGGAAAACTGGGTCTACCGAGGTGCTGATTGAGGCCGCACGGCTGGCTATTGAGGTCGCTAGGGTGGCT
TTCAAAGTCGGCAGTCCTGAAACTGCAAAGGAAGCAGTTCGCACCGCATTAGAACTTGTTAAGGAGCTGATAC
AACAGGCACTCAAGACCGGCAGTGACGAGGTTCTTGAGCGGGCTGCAGAGCTTGCTAAAGAGGTGGCAAGGGT
GGCTAAGGAAGTTGGAGATCCACGGGCCGCTCGCAAAGCAGATATGGTTGCCAAAATTGCCGATACCCTCCGC
GAACTTATGGAGTCCCTGAGGGAACTGCGCCGAATTCTCGAGGAACTTAAGGAAATGCTGGAGCGGCTCGAGA
AGAACCCTGACAAGGATGTGATTGTGAAGGTTCTTAAAGTGATCGTTAAAGCCATCGAAGCCTCTGTCGAAAA
CCAACGAATCTCCGCTGAGAACCAAGCTGCTTTGGCTTCATTGGCG(TGATGA)(CTCGAGCTGGTACTGCAT
GCACGCAATGCTAGCTGCCCCTTTCCCGTCCTGGGTACCCCGAGTCTCCCCCGACCTCGGGTCCCAGGTATGC
TCCCACCTCCACCTGCCCCACTCACCACCTCTGCTAGTTCCAGACACCTCCCAAGCACGCAGCAATGCAGCTC
AAAACGCTTAGCCTAGCCACACCCCCACGGGAAACAGCAGTGATTAACCTTTAGCAATAAACGAAAGTTTAAC
TAAGCTATACTAACCCCAGGGTTGGTCAATTTCGTGCCAGCCACACCCTGGAGCTAGC){AAAAAAAAAAAAA
AAAAAAAAAAAAAAAAAGCATATGACTAAAAAAAAAAAAAAAAAAAAAAAAAAAAAAAAAAAAAAAAAAAAAA
AAAAAAAAAAAAAAAAAAAAAAAA) (SEQ ID NO: 87)
Rpk9_RBD_SARS-CoV-2_KWOCA-96
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKLNDLCWTNIYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPLQSYGFQPTNGVGYQPYRVVV
LSFELLHAPATVCGPKKST(GGSGGSGSGGSGGSGS)GRELKQLAEVLEEIQRLAEEARKLMTDEEEAKKIQE
EAERAKRMLASAVWAVTDNEVIEKLLEVVKEIIRLAEEAMKKMTDEEEAAKIAKEALEAIKMLARAVEEVTDN
EVIEKLLEVVKEIIRAAEEAMKLMRNEEEAAKIAKKALEAIKALAEAVEAIKNKEEIERLLTLVKELIRKAEE
EARRMSDREKAAEIIERALEKIKKLAKLAKALADLEKALRELKKSIDELERSLEELERNPSEAALVENNRLNV
ENNKIIVEVLRIIAEVLKINAELA(SEQ ID NO: 88)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCTTTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTTCACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCGGCCGCGAACTGAAACAGCTTGCTGAGGTGCTCGAGGAAATTCAACGCCTGGCTGAGGAAGC
CCGCAAGCTGATGACAGACGAGGAGGAAGCCAAGAAGATTCAGGAGGAGGCCGAGCGCGCTAAGCGCATGCTG
GCATCCGCTGTTTGGGCCGTAACGGATAACGAAGTGATTGAGAAGCTCCTCGAGGTCGTGAAAGAGATCATCC
GCCTCGCCGAGGAGGCTATGAAGAAGATGACCGACGAGGAGGAAGCTGCCAAGATCGCAAAGGAGGCCCTCGA
GGCCATCAAAATGCTGGCTAGAGCCGTTGAGGAAGTGACAGATAATGAAGTAATTGAGAAGCTGCTCGAGGTG
GTCAAAGAGATTATCAGGGCCGCCGAGGAGGCCATGAAGCTCATGAGAAACGAAGAAGAGGCAGCTAAGATCG
CTAAGAAGGCTCTGGAGGCCATTAAAGCTCTCGCTGAGGCCGTGGAGGCTATCAAGAACAAGGAGGAGATTGA
GCGGCTTCTGACTCTTGTCAAGGAGCTGATCCGGAAAGCTGAGGAGGAAGCAAGGCGGATGAGCGACCGCGAG
AAAGCCGCTGAGATCATTGAGCGGGCACTCGAGAAAATTAAAAAGCTTGCAAAGCTGGCTAAAGCCCTGGCCG
ATCTTGAGAAAGCCCTGAGGGAACTCAAAAAGTCCCTCGACGAACTGGAGCGGAGTCTGGAAGAGCTCGAGCG
CAACCCTTCTGAGGCCGCCCTGGTTGAGAACAACCGCCTGAATGTCGAGAACAATAAGATCATCGTCGAGGTT
CTCCGGATCATTGCTGAGGTGCTCAAAATTAACGCCGAACTCGCG(TGATGA)(CTCGAGCTGGTACTGCATG
CACGCAATGCTAGCTGCCCCTTTCCCGTCCTGGGTACCCCGAGTCTCCCCCGACCTCGGGTCCCAGGTATGCT
CCCACCTCCACCTGCCCCACTCACCACCTCTGCTAGTTCCAGACACCTCCCAAGCACGCAGCAATGCAGCTCA
AAACGCTTAGCCTAGCCACACCCCCACGGGAAACAGCAGTGATTAACCTTTAGCAATAAACGAAAGTTTAACT
AAGCTATACTAACCCCAGGGTTGGTCAATTTCGTGCCAGCCACACCCTGGAGCTAGC)(AAAAAAAAAAAAAA
AAAAAAAAAAAAAAAAGCATATGACTAAAAAAAAAAAAAAAAAAAAAAAAAAAAAAAAAAAAAAAAAAAAAAA
AAAAAAAAAAAAAAAAAAAAAAA) (SEQ ID NO: 89)
Rpk9_RBD_SARS-CoV-2_KWOCA-101
Amino acid:
(MGILPSPGMPALLSLVSLLSVLLMGCVAETGT)RFPNITNLCPFGEVFNATRFASVYAWNRKRISNCVADFS
VLYNSASFSTFKCYGVSPTKLNDLCWTNIYADSFVIRGDEVRQIAPGQTGKIADYNYKLPDDFTGCVIAWNSN
NLDSKVGGNYNYLYRLFRKSNLKPFERDISTEIYOAGSTPCNGVEGFNCYFPLQSYGFQPTNGVGYQPYRVVV
LSFELLHAPATVCGPKKST(GGSGGSGSGGSGGSGS)SDEEEAREWAERAEEAAKEALEQAKREGDEIARLCA
EMLEILAEEARRKKDSEEAEAVYWAARATLAALEALEOAKREGDEDARRCAEELLRLACSAAARQDSEQARAV
YEAARAVLAALRALEAAKRAGMEEARKEAEELLRRACEAARKQDPELARAVRDKAELLKALADLFKALKELKK
SLDELERSLEELEKNPSEDALVENNRINVENNKIIVEVLRIIAEVLRINARAV (SEQ ID NO: 90)
DNA:
(AG)(GAATAAACTAGTATTCTTCTGGTCCCCACAGACTCAGAGAGAACCCGCCACC)ATGGGCATCCTGCCA
AGCCCTGGAATGCCTGCCCTGCTGAGCCTGGTGTCCCTGCTGTCTGTGCTGCTGATGGGATGCGTGGCAGAGA
CCGGCACAAGGTTCCCTAACATCACCAATCTGTGCCCATTCGGCGAGGTGTTTAACGCCACACGCITTGCCTC
CGTGTATGCCTGGAACCGGAAGAGAATCTCTAATTGCGTGGCCGACTTCAGCGTGCTGTACAATAGCGCCTCC
TTCTCTACCTTTAAGTGCTATGGCGTGAGCCCCACCAAGCTGAACGATCTGTGCTGGACAAACATTTACGCCG
ACTCCTTTGTGATCCGGGGCGATGAAGTGAGACAGATCGCACCAGGACAGACCGGAAAGATCGCAGACTACAA
CTATAAGCTGCCTGACGATTICACAGGCTGCGTGATCGCCTGGAATAGCAACAATCTGGATTCCAAAGTGGGC
GGCAACTACAATTATCTGTACAGGCTGTTCCGCAAGTCCAACCTGAAGCCATTTGAGCGGGACATCTCCACCG
AGATCTACCAGGCCGGCTCTACACCCTGCAACGGCGTGGAGGGCTTCAATTGTTATTTTCCCCTGCAGTCTTA
CGGCTTCCAGCCTACAAATGGCGTGGGCTATCAGCCATACCGGGTGGTGGTGCTGTCTTTTGAGCTGCTGCAC
GCACCAGCAACCGTGTGCGGCCCCAAGAAGAGCACAGGAGGCTCTGGAGGCAGCGGCTCCGGAGGCTCTGGAG
GCAGCGGCTCCTCCGACGAAGAGGAAGCTAGAGAATGGGCCGAAAGAGCAGAAGAGGCAGCCAAGGAGGCATT
GGAACAAGCTAAGCGCGAAGGGGATGAAATCGCTAGACTTTGCGCTGAAATGCTTGAGATCCTGGCCGAAGAG
GCTAGGCGCAAGAAGGACTCTGAGGAGGCCGAGGCAGTGTATTGGGCCGCTAGAGCCACCCTGGCTGCTCTGG
AAGCACTGGAACAGGCTAAACGCGAGGGCGATGAGGACGCTAGGCGCTGTGCTGAGGAACTCCTTAGACTCGC
CTGTAGTGCAGCCGCACGGCAGGACTCTGAGCAAGCAAGGGCAGTTTATGAGGCTGCTAGGGCTGTTTTGGCC
GCCCTGCGGGCTCTTGAGGCTGCTAAACGGGCTGGGATGGAGGAAGCCCGCAAGGAAGCTGAGGAACTGCTGA
GACGCGCCTGTGAAGCTGCTAGAAAGCAGGATCCCGAACTTGCGAGGGCTGTTCGCGATAAAGCAGAGCTTCT
GAAGGCACTGGCCGATCTCTTCAAGGCTCTCAAGGAACTCAAAAAGTCTCTGGATGAGCTCGAACGATCTCTG
GAGGAGCTGGAGAAGAATCCTTCCGAGGACGCACTCGTCGAGAACAATAGACTGAATGTTGAGAATAACAAGA
TCATTGTTGAAGTTCTACGGATTATCGCTGAGGTCTTGCGCATTAATGCAAGAGCCGTG(TGATGA)(CTCGA
GCTGGTACTGCATGCACGCAATGCTAGCTGCCCCTTTCCCGTCCTGGGTACCCCGAGTCTCCCCCGACCTCGG
GTCCCAGGTATGCTCCCACCTCCACCTGCCCCACTCACCACCTCTGCTAGTTCCAGACACCTCCCAAGCACGC
AGCAATGCAGCTCAAAACGCTTAGCCTAGCCACACCCCCACGGGAAACAGCAGTGATTAACCTTTAGCAATAA
ACGAAAGTTTAACTAAGCTATACTAACCCCAGGGTTGGTCAATTTCGTGCCAGCCACACCCTGGAGCTAGC)
{AAAAAAAAAAAAAAAAAAAAAAAAAAAAAAGCATATGACTAAAAAAAAAAAAAAAAAAAAAAAAAAAAAAAAA
AAAAAAAAAAAAAAAAAAAAAAAAAAAAAAAAAAAAA) (SEQ ID NO: 91)

In another aspect, the disclosure provides nucleic acids encoding a polypeptide or fusion protein of the disclosure. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In one embodiment, the nucleic acid comprises mRNA. The mRNA may be modified as appropriate, for example, for use as a vaccine. In one embodiment, the RNA comprises nucleoside-modified RNA, including but not limited to NI-methylpseudouridine-5′-triphosphate containing RNA. In another embodiment, the mRNA comprises self-amplifying mRNA.

In a further embodiment, the nucleic acid encodes a poly A tail (DNA) or comprises a poly A tail (RNA). In another embodiment, the nucleic acid encodes a 5′ UTR and/or a 3′ UTR (DNA) or comprises a S′ UTR and/or a 3′ UTR (RNA).

In exemplary embodiments, the nucleic acid comprises the sequence selected from SEQ ID NO: 73, 75, 77, 79, 81, 83, 85, 87, 89, and 91, wherein residues in parentheses are optional and may be present or may be deleted, or an RNA expression product thereof.

In another aspect, disclosure provides expression vectors comprising the nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence is still considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In one aspect, the present disclosure provides cells comprising the polypeptide, the nanoparticle, the composition, the nucleic acid, and/or the expression vector of any embodiment or combination of embodiments of the disclosure, wherein the cells can be cither prokaryotic or eukaryotic, such as mammalian cells. In one embodiment, the cells may be transiently or stably transfected with the nucleic acids or expression vectors of the disclosure. Such transfection of expression vectors into prokaryotic and eukaryotic cells can be accomplished via any technique known in the art. A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

In a further aspect, the disclosure provides nanoparticle comprising a plurality of the polypeptides and/or the fusion proteins of any embodiment or combination of embodiments of the polypeptides of the invention. As is disclosed herein, the polypeptides and fusion proteins of the disclosure are capable of self-assembling into trimers. The nanoparticles can be used for any purpose, including antigen display and as a vaccine. In some embodiments, all of the polypeptides or fusion proteins are fused to a polypeptide antigen, wherein the polypeptide antigen may be identical in all of the polypeptides or fusion proteins, or wherein the nanoparticle may present more than one polypeptide antigen. In other embodiments, only a portion of the polypeptides or fusion proteins are fused to a polypeptide antigen, wherein the polypeptide antigen present may be identical in all cases, or wherein the nanoparticle may present more than one polypeptide antigen.

In a further aspect, the disclosure provides pharmaceutical compositions/vaccines comprising
(a) the polypeptide, fusion protein, nanoparticle, nucleic acid, expression vector, and/or cell of any embodiment or combination of embodiments herein; and
(b) a pharmaceutically acceptable carrier.

The compositions/vaccines may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The compositions/vaccines may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen. Exemplary adjuvants include, but are not limited to, Adju-Phos™, Adjumer™, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin Al-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in peDNA3, IL-2/Ig plasmid, IL-2/Ig protein, IL-4, IL-4 in pcDNA3, Imiquimod™, Imm Ther™ Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT (R192G), LT-OA or LT Oral Adjuvant, LT-R192G, LTK63, LTK72, MF59, MONTANIDE ISA 51, MONTANIDE ISA 720, MPL. TM., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCATI, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

In some embodiments, the pharmaceutical composition or vaccine comprises a nucleic acid encoding a polypeptide or fusion protein of any embodiment herein. In some such embodiments, the pharmaceutically acceptable carrier comprises a cationic lipid such as a liposome, or a cationic protein such as protamine.

In a further aspect, the disclosure provides methods to treat or limit development of a infection, comprising administering to a subject in need thereof an amount effective to treat or limit development of the infection of the fusion protein, nanoparticle comprising the fusion protein, nucleic acid encoding a fusion protein, an expression vector comprising the nucleic acid, a cell comprising the fusion protein, nucleic acid, or expression vector; and/or a pharmaceutical composition or vaccine comprising the fusion protein, nucleic acid, expression vector, of any embodiment herein (referred to as the “immunogenic composition”). The subject may be any suitable mammalian subject, including but not limited to a human subject.

The infection may be any infection that the fusion protein includes an antigen that an immune response against could be used to treat or limit development of the infection. In one embodiment, the infection is a SARS COV-2 infection.

When the method comprises limiting a SARS-COV-2 infection, the immunogenic composition is administered prophylactically to a subject that is not known to be infected, but may be at risk of exposure to SARS-COV-2. As used herein, “limiting development” includes, but is not limited to accomplishing one or more of the following: (a) generating an immune response (antibody and/or cell-based) to of SARS-COV-2 in the subject; (b) generating neutralizing antibodies against SARS-COV-2 in the subject (b) limiting build-up of SARS-COV-2 titer in the subject after exposure to SARS-CoV-2; and/or (c) limiting or preventing development of SARS-COV-2 symptoms after infection. Exemplary symptoms of SARS-COV-2 infection include, but are not limited to, fever, fatigue, cough, shortness of breath, chest pressure and/or pain, loss or diminution of the sense of smell, loss or diminution of the sense of taste, and respiratory issues including but not limited to pneumonia, bronchitis, severe acute respiratory syndrome (SARS), and upper and lower respiratory tract infections.

In one embodiment, the methods generate an immune response in a subject in the subject not known to be infected with SARS-COV-2, wherein the immune response serves to limit development of infection and symptoms of a SARS-COV-2 infection. In one embodiment, the immune response comprises generation of neutralizing antibodies against SARS-COV-2. In a further embodiment, the immune response comprises generation of antibodies against multiple antigenic epitopes.

As used herein, an “effective amount” refers to an amount of the immunogenic composition that is effective for treating and/or limiting SARS-COV-2 infection. The polypeptide, nanoparticle, composition, nucleic acid, pharmaceutical composition, or vaccine of any embodiment herein are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 μg/kg-100 mg/kg body weight of the polypeptide or nanoparticle thereof. The composition can be delivered in a single bolus, or may be administered more than once (e.g., 2, 3, 4, 5, or more times) as determined by attending medical personnel.

In one embodiment, the administering comprises administering a first dose and a second dose of the immunogenic composition, wherein the second dose is administered about 2 weeks to about 12 weeks, or about 4 weeks to about 12 weeks after the first does is administered. In various further embodiments, the second dose is administered about 2, 3, 4, 5, 6, 7, 8, 9, 10, 11, or 12 weeks after the first dose. In another embodiment, three doses may be administered, with a second dose administered about 1, 2, 3, 4, 5, 6, 7, 8, 9, 10, 11, or 12 weeks after the first dose, and the third dose administered about 1, 2, 3, 4, 5, 6, 7, 8, 9, 10, 11, or 12 weeks after the second dose.

In another embodiment of the methods, the subject is infected with a severe acute respiratory (SARS) virus, including but not limited to SARS-COV-2, wherein the administering elicits an immune response against the SARS virus in the subject that treats a SARS virus infection in the subject. When the method comprises treating a SARS-COV-2 infection, the immunogenic compositions are administered to a subject that has already been infected with SARS-COV-2, and/or who is suffering from symptoms (as described above) indicating that the subject is likely to have been infected with SARS-COV-2.

As used herein, “treat” or “treating” includes, but is not limited to accomplishing one or more of the following: (a) reducing SARS-COV-2 titer in the subject; (b) limiting any increase of SARS-COV-2 titer in the subject; (c) reducing the severity of SARS-COV-2 symptoms; (d) limiting or preventing development of SARS-COV-2 symptoms after infection; (e) inhibiting worsening of SARS-COV-2 symptoms; (f) limiting or preventing recurrence of SARS-COV-2 symptoms in subjects that were previously symptomatic for SARS-COV-2 infection; and/or (e) survival.

In another aspect, the disclosure provides methods for generating an immune response in a subject, comprising administering to the subject an amount effective to generate an immune response of the fusion protein of any embodiment or combination of embodiments herein, a nucleic acid encoding the fusion protein, an expression vector comprising the nucleic acid, a cell comprising the fusion protein, nucleic acid, or expression vector; and/or a pharmaceutical composition comprising the fusion protein, nucleic acid, expression vector, or cell.

### EXAMPLE 1

### Introduction

Secreted proteins make up nearly 20% of the human proteome, and are the primary method of intercellular communication in animals (Uhlen et al. 2019; Farhan and Rabouille 2011). Due to their potent and wide-ranging functions, many secreted proteins, such as antibodies, hormones, cytokines, and growth factors, are of great interest for therapeutic applications. Furthermore, secreted or membrane-anchored proteins from pathogens are often targets for prophylactic or therapeutic interventions in infectious disease. Secretion from eukaryotic cells is required for the recombinant production of many protein biologics, as they often feature secretory pathway-specific post-translational modifications such as furin-mediated proteolytic cleavage (Braun and Sauter 2019), glycosylation (Ohtsubo and Marth 2006), and disulfide bond formation (Wittrup 1995). Understanding and controlling the secretion of a protein of interest is thus mandatory for the development of secreted protein technologies.

We developed a general computational protocol, named the Degreaser that specifically designs away cryptic transmembrane domains without sacrificing overall structural stability. We demonstrate the ability of the Degreaser to avoid the introduction of cryptic transmembrane domains during the design of a new set of robustly secreting designed protein nanoparticles.

### Results

### De Novo Design of Secretion-Optimized One-Component Protein Assemblies

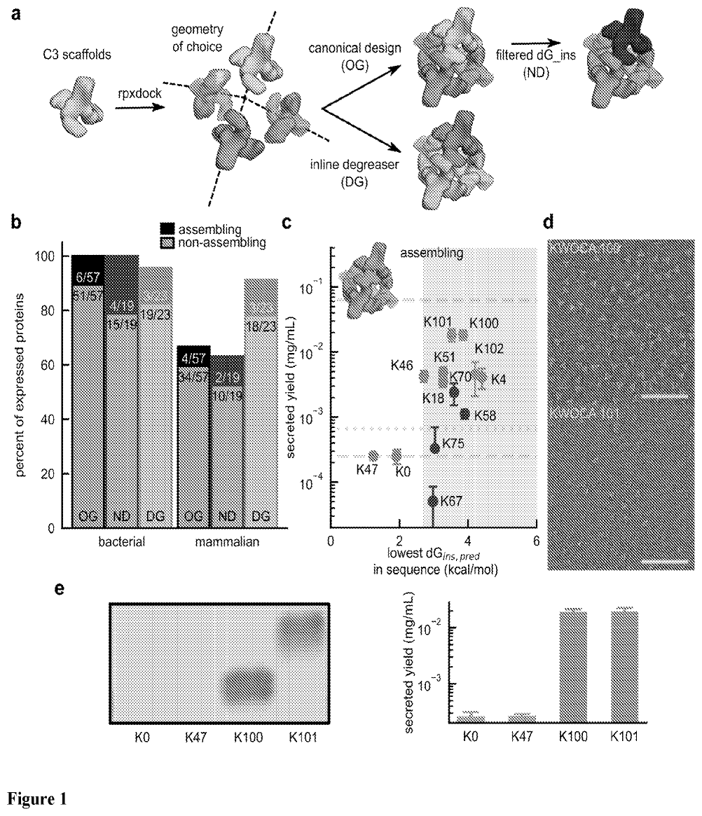

Given the success of the Degreaser in retroactively improving the secretion of several nanoparticle components and a computationally designed nanoparticle, we next tested its prospective use and compatibility with large-scale design protocols by incorporating it into the design of a set of new one-component nanoparticles intended to secrete robustly from mammalian cells. We used as building blocks a set of 1,094 models of trimeric proteins consisting of de novo helical bundles fused to designed helical repeat proteins as previously described (Hsia et al. 2021). These building blocks were docked as rigid bodies into three target architectures containing three-fold symmetry axes: icosahedral (13), octahedral (03), and tetrahedral (T3) (FIG. 1a). After docking, residues at interfaces with adjacent building blocks were designed using Rosetta™ to enable spontaneous self-assembly to the target architecture. Three fully automated design protocols were compared: OG, ND, and DG (FIG. 1a). The OG protocol used a conventional, dG_{ins,pred}-agnostic protocol and therefore generated designs that had dG_{ins,pred} values both above and below +2.7 kcal/mol. The ND protocol simply applied a post-design filter to the OG design set that rejected any designs with dG_{ins,pred} less than +2.7 kcal/mol. Finally, the DG protocol incorporated the Degreaser after the interface design step without changing any other protocol steps or design parameters, further filtering out any designs with dG_{ins,pred} less than +2.7 kcal/mol. Taking into account our previous finding that decreasing the hydrophobicity of the lowest dG_{ins,pred} segment in a given protein maximized improvement of protein secretion, only variants following this rule were considered. In this benchmark design set, we still used the conservative approach of allowing the Degreaser to change at maximum one residue per design. It is important to note that not all DG designs harbor a Degreaser mutation, as designs that do not have segments of low dG_{ins,pred} pass the Degreaser step and are accepted without modification.

Importantly, the incorporation of Degreaser-guided design into an otherwise conventional design protocol did not substantially perturb the structural metrics typically used to gauge the quality of designed nanoparticle interfaces. Within the DG design set, 420 of the 1,048 designs (40%) were actually mutated by the Degreaser, while mutations meeting the Degreaser criteria were not identified for 18 designs and these were rejected. After filtering, DG designs that were not mutated by the Degreaser had an average dG_{ins,pred} of +3.97 kcal/mol, while those bearing mutations had an average dG_{ins,pred} of +3.38 kcal/mol. Because sequences with originally high dG_{ins,pred} are not mutated, the lower average dG_{ins,pred} of sequences with mutations is due to the low original dG_{ins,pred} of those sequences. A slight shift in the distribution of ddG (the predicted energy of interface formation) was observed, which was expected due to Degreaser-introduced polar residues in what remained predominantly hydrophobic interfaces, accompanied by a small shift in the distribution of the interface shape complementarity (sc). On the other band, the buried solvent-accessible surface area (sasa) showed a nearly identical distribution to that of conventional (OG) and dG_{ins,pred} filtered (ND) designs. After filtering on several structural metrics and visual inspection of the top-scoring designs by ddG, we selected 99 KWOCAs (Khmelinskaia-Wang one-component assemblies) for experimental characterization. These included 57 OG, 19 ND, and 23 DG designs, which differed in dG_{ins,pred} but not other structural metrics (data not shown). 8 of the selected DG designs included mutations introduced by the Degreaser.

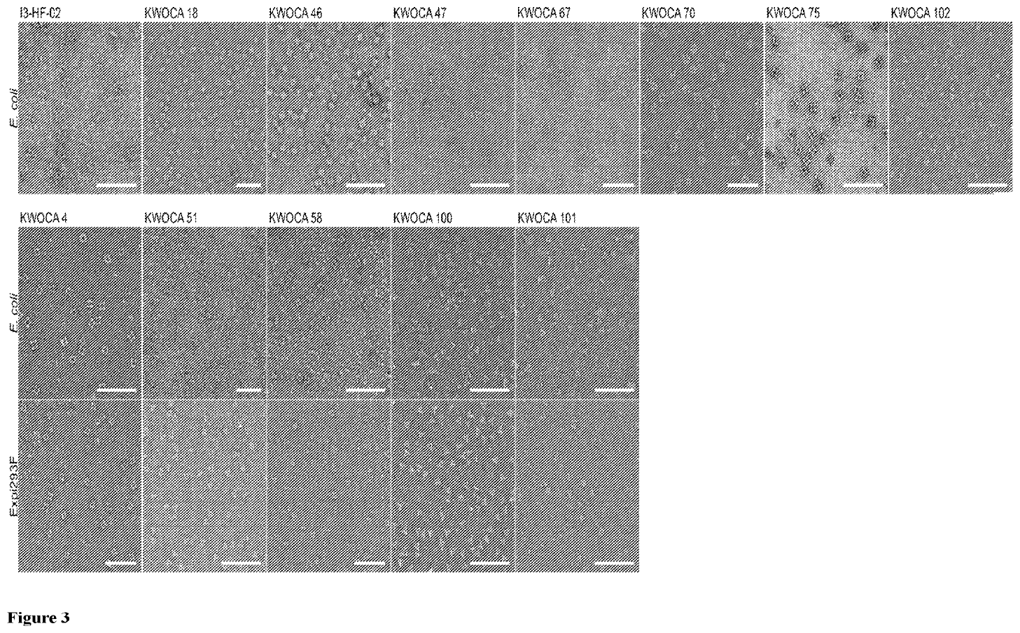

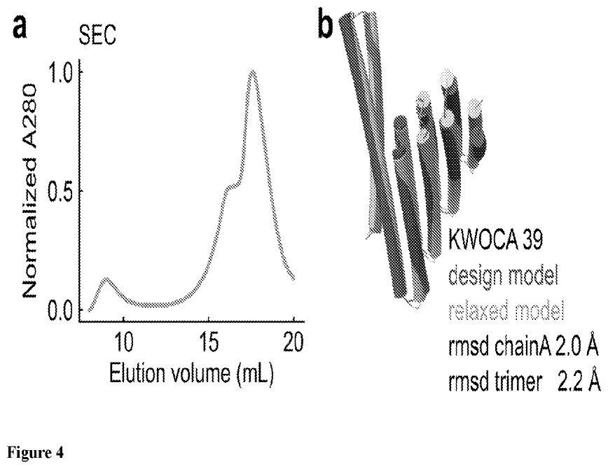

We first expressed the KWOCAs in the cytoplasm of E. coli to determine which ones successfully assembled to the intended structures regardless of secretion from mammalian cells. All KWOCAs but one yielded sufficient protein for purification and characterization in the soluble fraction of clarified E. coli lysates (FIG. 1c). During SEC purification, 22 of 99 KWOCAs yielded a peak with an elution volume corresponding to a protein complex larger than that expected for a trimer, but smaller than unbounded aggregates. Most of these also showed peaks at elution volumes corresponding to unassembled trimeric protein. DLS of fractions from the early peaks indicated that the majority of these 22 designs formed monodisperse assemblies, and nsEM confirmed that 13 assembled to homogeneous nanoparticle structures (FIG. 1c and FIG. 3). Crystallization of several KWOCAs resulted in high-resolution structures of trimeric scaffolds of one non-assembling (KWOCA 39), three non-confirmed assemblies (KWOCA 60, 65 and 73) and one confirmed assembling KWOCA (KWOCA 102) (FIG. 4). The obtained crystal structures matched closely the monomeric subunit of the design models (1.0 to 2.2 Å Ca rmsd), with larger deviations observed on the trimer level (2.2 to 4.0 Å Ca rmsd). These differences suggest that various design regions and structural features, such as the helical bundle interface, the flexibility in the designed helical repeat domains and their fusion to the trimeric helical bundles, are the source of subtle structural errors that propagate across the assembly. Surprisingly, the degree of error does not seem to correlate with the assembly success.

We next evaluated secretion of the KWOCAs from transfected Expi293F cells by measuring the levels of myc-tagged protein in clarified harvest fluid by western blot (see Methods). A majority of the KWOCAs (68%) secreted with greater yield than 13-01, our benchmark modestly secreted protein nanoparticle (FIG. 1e). The higher success in secretion within the DG KWOCA set (87% compared to 67% and 48% in the OG and ND sets, respectively) confirm the utility of the Degreaser in predicting and improving protein secretion. For further analysis, we separated the experimentally characterized KWOCAs into two categories: non-assembling proteins (FIG. 4) and confirmed assemblies (FIG. 3). The non-assembling proteins show only a weak trend of higher secretion yield with higher dG_{ins,pred}, though this is confounded by the varying overall expression levels of these proteins. Although we did not observe consistent differences in secretion yield among the OG, ND, and DG sets of non-assembling proteins, inline application of the Degreaser (DG) tended to provide a greater benefit to secreted yield than filtering on dG_{ins,pred} after conventional design (ND). Of the 13 EM-validated assembling designs (FIG. 3), 8 secrete at higher levels than the original 13-01 design, and the highest secreted yield (KWOCA 101) was within two-fold of the highest-secreting redesigned variant of 13-01 (13-01NS). Characterization of these 8 KWOCAs by SEC, DLS and nsEM revealed that each was indistinguishable from its bacterially produced counterpart (FIG. 3). All but one of the verified assemblies (KWOCA 47, from the OG design set) have no segments of low dG_{ins,pred} (<2.7 kcal/mol), suggesting that avoiding cryptic transmembrane domains is more important for the productive secretion of self-assembling compared to non-assembling proteins. Intriguingly, inline application of the Degreaser led to the highest yield of secreted nanoparticles (KWOCAs 100, 101, and 102), although the proportion of confirmed assemblies was not significantly different between conventionally designed proteins (including those filtered on dG_{ins,pred}) and Degreaser-designed candidates (12/76 (16%) vs. 3/23 (13%), respectively; FIG. 1c). These data indicate that the Degreaser can be applied to improve secreted yield from mammalian cells while maintaining a similar success rate in design outcome.

Comparison of several pairs of closely related designs yielded additional insights into secretion determinants. For example, KWOCA 51 and 101, which form closely related tetrahedral assemblies, used the same input scaffold for design and differ by only two residues. However, KWOCA 101 has a higher lowest dG_{ins,pred} and secreted with a roughly four-fold greater yield than KWOCA 51, substantiating the idea that small changes in designed protein sequences can lead to considerable changes in secreted yield. Also related are the Degreased KWOCA 100 and the conventionally-designed KWOCA 46, both confirmed assemblies (FIG. 3), in which a one-residue difference led to a +1.13 kcal/mol change in dG_{ins,pred} and a five-fold increase in secretion. In both of these cases, the conventional design pipeline would have resulted in assemblies that secrete poorly, requiring retrospective application of the Degreaser. Two other pairs of designs suggested that assembly state may affect secreted yield. The non-assembling KWOCA 88 differs from the octahedral KWOCA 4 by only two residues, but KWOCA 88 secretes with a roughly four-fold higher yield. Finally, even though KWOCA 73 differs from KWOCA 41 by only two residues, the former showed higher-order material by SEC and DLS whereas the latter did not (FIG. 4), and KWOCA 73 secretes at about half the yield of KWOCA 41 even though its lowest dG_{ins,pred} value is much higher. Together our data suggest that, although there appears to be a general secretion penalty for self-assembling proteins, in-line use of the Degreaser during design can improve secreted nanoparticle yield.

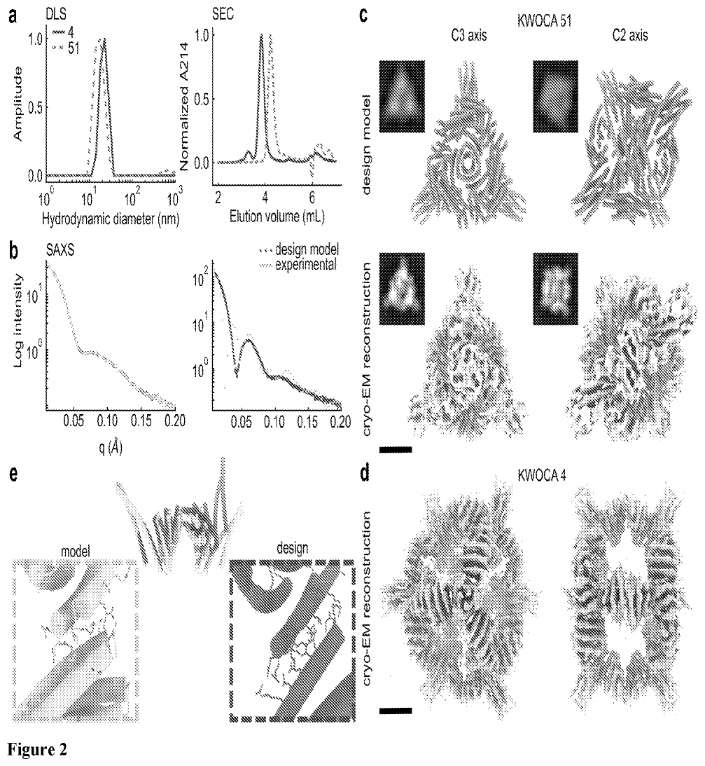

We obtained single-particle cryoEM reconstructions of two highly secreted assemblies, KWOCA 51 and KWOCA 4, to evaluate our design protocol at high resolution. DLS and SEC indicated that both designs assemble into monodisperse nanoparticles, with KWOCA 51 forming a smaller particle than KWOCA 4 (˜19 and 26 nm hydrodynamic diameter, respectively) as expected by design (˜17 and 32 nm, respectively) (FIG. 2b,c). Comparing calculated to experimental SAXS profiles further revealed that KWOCA 51 homogeneously assembles into the intended tetrahedral geometry, while KWOCA 4 significantly deviates from the design model (FIG. 2d). Indeed a single-particle cryo-EM reconstruction of KWOCA 51 at 5.1 Å resolution closely matched the design model, and relaxing the model into the density led to only minor deviations within each subunit (1.3 Å Ca rmsd) that mainly reflect slight structural flexibility of the helical repeat domain (FIG. 2 a,d). In contrast, a cryo-EM map of KWOCA 4 at 6.6 Å resolution revealed that the protein does not form the computationally designed icosahedral assembly, instead identifying an octahedral nanoparticle as the only species present in the assembly fraction from SEC. Accordingly, a SAXS profile calculated from a cryo-EM model obtained by fitting and relaxing trimeric building blocks into the density closely matched the experimental data. Interestingly, only minor structural deviations within the trimeric building blocks were observed when comparing the computational design model to the relaxed cryo-EM model (1,3 Å Ca rmsd), indicating that the off-target assembly must be due to differences in the computationally designed interface between the trimers. Indeed, the angle between two contiguous subunits in the cryo-EM model is rotated by 27°, resulting in a deviation of 18 Å of the contiguous subunit compared to the design model. This rotation is further accompanied by a 3 Å transverse translation of the center of mass of the designed interface past the C2 symmetry axis, suggesting that the edge residues of the originally designed interface were loosely packed and only weakly contributing to the interface energy. To our knowledge, this is the first report of a high-resolution structure of a de novo computationally designed protein nanoparticle that forms a well-defined architecture distinct from the one intended. Nevertheless, the two structures together establish that KWOCAs 4 and 51, which secrete from mammalian culture at higher levels than lumazine synthase and 13-01, are highly ordered monodisperse de novo designed nanoparticles.

### DISCUSSION

Computational protein design methodologies are advancing rapidly, enabling the exploration of previously unexplored spaces in protein structure and function (Huang, Boyken, and Baker 2016; Baek and Baker 2022). In addition to increasing our fundamental understanding of proteins, these advances have brought commercial application of computationally designed proteins within reach. For example, computationally designed cytokine mimetics (Silva et al. 2019), enzymes for gluten degradation (Gordon et al. 2012), and nanoparticle vaccines (Marcandalli et al. 2019; Boyoglu-Barnum et al. 2021; Walls et al. 2020) have recently advanced to clinical trials. As designed proteins become increasingly useful, methods for optimizing various phenotypes other than structure and stability become more important. The Degreaser was explicitly constructed to be modular—as showcased by our redesign of existing proteins as well as our application of the Degreaser in-line during the design of new secretable protein assemblies—while preserving structural stability and integrity. These features enable its application to any protein. Furthermore, application of the Degreaser in-line during design is minimally invasive: it only mutates proteins that require elimination of cryptic transmembrane domains, and it identifies the minimal sufficient perturbation. As we showed during KWOCA design, this approach allows in-line implementation of the Degreaser that should eliminate the requirement for retroactive redesign of poorly secreting proteins.

More broadly, any method for improving the yield of recombinant biologics is valuable. For example, the decades of effort invested in optimizing and industrializing the production of monoclonal antibodies now underpins the biologics industry (Kelley 2009). Methods like the Degreaser that encode improved yield or performance in the sequence of the molecule itself are especially desirable, as they make the improvements “automatic”: they do not require other actions like the use of specialized cell culture media or co-transfection of chaperones. The Degreaser and the new highly secretable KWOCAs we describe here can be used in mRNA-launched nanoparticle vaccines with atomic-level accuracy. This approach enables structural and functional optimization of the nanoparticle scaffolds in ways that are not possible when relying on naturally occurring scaffolds.

### Methods

In the examples of Degreaser-guided protein nanoparticle (re) design here discussed, we allowed only one mutation per input structure. Although only interfacial residues were allowed to design within the conventional design protocol, the Degreaser is allowed to change any of the residues it identifies to be within hydrophobic segments. However, the Degreaser can be specified to only operate on a subset of residues within a given model, much as any other Mover can be. By allowing only one mutation, our goal was to minimally distrub the interfaces resulting from conventional design, which contain between 7 and 24 residues that participate in the hydrophobic interface and may not be able to easily accommodate several mutations. However, the Degreaser is amenable to allowing an arbitrary number of mutations per hydrophobic segment. Furthermore, not every hydrophobic segment identified is in the vicinity of the designed interface. Both considerations warrant further investigation.

Computational design of protein nanoparticles Trimeric scaffolds were generated by helical fusion of previously designed trimeric helical bundles (Boyken et al. 2019) and de novo helical repeat domains (Brunette et al. 2015), following the protocol described in (Hsia et al. 2021). Symmetrical docking of the top scoring 1094 trimmers was performed using the rpdock protocol. Briefly, the 3-fold symmetry axis of the trimeric scaffolds was aligned with that of one of the target symmetries: I, O, or T. These aligned trimers may rotate around and translate along their respective symmetry axis while maintaining the symmetry of the complex (King et al. 2012, 2014). These two degrees of freedom, radial displacement and axial rotation, were sampled in increments of 1 A and 1°, respectively. For each docked configuration in which no clashes between the backbone and beta carbon atoms of adjacent building blocks were present, an RPX designability score was calculated (Fallas et al. 2017). High-scoring docked configurations with intermediately sized interfaces (ncontacts <75) were selected for full-atom interface design using Rosetta™ scripts as previously described (King et al. 2014; Hsia et al. 2016). Briefly, the design protocol took a single-chain input pdb and a symmetry definition file containing information for a specified cubic point group symmetry (DiMaio et al. 2011). The oligomers were then aligned to the corresponding axes of the symmetry using the Rosetta™ SymDofMover, taking into account the rigid body translations and rotations retrieved from the pickle file output from the docking protocol (King et al. 2014; Hsia et al. 2016). The conventional symmetric interface design protocol was modified for Degreaser in-line design, by adding the Degreaser Mover step after the final step of conventional design, before any filters were applied to a particular docked model. Individual design trajectories were filtered by the following criteria: difference between Rosetta™ energy of bound and unbound states less than −20.0 REU interface surface area greater than 500 A2, sc greater than 0.6, and at least three helices at the interface. Designs arising from the conventional design protocol were further filtered with dG_{ins,pred} >2.7, making up the ND pool. Designs that passed these criteria were manually inspected and a set of 99 designs selected for experimental characterization: 57 OG, 19 ND, and 23 DG.

### Cell Culture, Protein Expression and Purification

All bacterial protein expression was performed with Lemo21 (DE3) competent E. coli (NEB), all bacterial plasmid propagation with NEB 5-alpha competent E. coli (NEB), and all mammalian protein expression with Expi293F cells (ThermoFisher Scientific). All bacterial expression was performed from a pET29b (+) vector with genes between the Ndel and Xhol restriction sites. All mammalian expression was performed from a pCMV/R vector (Barouch et al. 2005) with genes between the Xbal and AvrII restriction sites, and all constructs used the same IgGx secretion signal. 6xHis tags for purification, myc tags for detection, as well as GS linkers and a photoactive Trp were placed at N-or C-termini of constructs as determined by available 3D space after manual inspection of design models (complete lists of gene and protein sequences can be found in Tables 1-2).

For purification of plasmid DNA for transfection, bacteria were cultured and plasmids were harvested according to the QIAGEN Plasmid Plus™ Maxi Kit protocol (QIAGEN). For bacterial expression and purification of previously-described nanoparticle component proteins, see previously-described methods (Bale et al. 2016; Hsia et al. 2016; Ueda et al. 2020b). For bacterial expression and purification of KWOCAs, proteins were expressed by autoinduction using TB11 media (Mpbio) supplemented with 50×5052, 20 mM MgSO4 and trace metal mix, under antibiotics selection at 18 degrees for 24 h after initial growth for 6-8 h at 37° C. Cells were harvested by centrifugation at 4000×g and lysed by sonication or microfluidization after resuspension in lysis buffer (50 mM Tris pH 8.0, 250 mM NaCl, 20 mM imidazole, 5% glycerol), followed by addition of Bovine pancreas DNasel (Sigma-Aldrich) and protease inhibitors (Thermo Scientific). Cells were lysed by sonication or by microfluidization. Clarified lysate supernatants were batch bound with equilibrated Ni-NTA resin (QIAGEN). Washes were performed with 5-10 column volumes of lysis buffer, then eluted with 3 column volumes of the same buffer containing 500 mM imidazole. Concentrated or unconcentrated eluted fractions were further purified using a Superose™ 6 Increase 10/300 GL (Cytiva) on an AKTA Pure™ (Cytiva) into 25 mM Tris pH 8.0, 150 mM NaCl, 5% glycerol. Instrument control and elution profiles analysis were performed with Cytiva software (Cytiva).

For mammalian expression and purification of KWOCAs and other secreted proteins, Expi293F cells were passaged according to manufacturer protocols (ThermoFisher Scientific). Cells at 3.0×10⁶ cells/mL were transfected with 1 μg/mL cell culture of purified plasmid DNA with 3 μg/μg PEI-MAX in 70 μL/mL of culture. For secretion yield measurements, cells were harvested at 72 h post-transfection by centrifugation for 5 minutes at 1,500 g. For protein purification, cells were harvested at 120 h post-transfection by centrifugation of cells and subsequent sterile filtering of supernatant. Filtered supernatant was adjusted to 50 mM Tris pH 8.0 and 500 mM NaCl, then bound to Ni Sepharose™ Excel (Cytiva) with agitation overnight. Pelleted resin was washed with 50 mM Tris pH 8.0, 500 mM NaCl, 30 mM imidazole, then eluted with the same buffer containing 300 mM imidazole. Concentrated elution fractions were purified by size-exclusion chromatography as described above.

Protein content and purity at each step of expression and purification were analyzed by SDS-PAGE using Criterion precast gels and electrophoresis systems (BIO-RAD). Purified protein concentration measurements were measured using UV absorbance at 280 nm, and calculated using theoretical molar extinction coefficients (ExPasy). Proteins were concentrated with 30,000 MWCO concentrators (Millipore). Purified, concentrated, and buffer-exchanged proteins were snap-frozen in liquid nitrogen and stored at −80° C. only if aggregates were absent as detected by DLS.

### Secretion Yield Quantification

Cells were harvested at 72 h post-transfection because maximal signal was present in cell supernatant while cell viability was still high (data not shown). Cells were centrifuged to separate medium from cells, and pelleted cells were resuspended in the same volume of removed medium in phosphate-buffered saline (PBS). All samples were then treated for min at 37° C. with 0.05% Triton-X™ 100 (Sigma) containing a 1:400 dilution of Benzonase endonuclease (EMD Millipore) to permeate membranes and prevent nucleic acid aggregation, which makes quantitative gel loading difficult. Internal myc-tag protein standard was also added at this point at 0.06 mg/mL. Treated samples were then diluted into 4X SDS 10 loading buffer (200 mM Tris pH 6.8, 40% glycerol, 8% SDS, bromophenol blue, 4 mM DTT) and incubated at 95° C. for 5 min. 14.3 μL of boiled samples were loaded onto Criterion 4-20% precast polyacrylamide gels (BIO-RAD). Precision Plus™ WesternC standards were included in each gel (BIO-RAD). Gels were run using BIO-RAD Criterion™ gel boxes and power supplies, then transferred using the Trans-Blot Turbo™ system onto 0.2 μm nitrocellulose membranes according to manufacturer instructions (BIO-RAD). Transferred blots were blocked in 3% milk in wash buffer (10 mM Tris pH 8.0, 150 mM NaCl, 0.1% Tween-20) for 30 min, then incubated with a 1:20,000 dilution of mouse anti-myc tag antibody (9B11, Cell Signaling Technology) with agitation, either 75 min at room temperature or 16 h at 4° C. Blots were then washed three times with wash buffer, then incubated 75 min at room temperature with a 1:10,000 dilution of goat anti-mouse HRP conjugated antibody (Cell Signaling Technology). After three washes with wash buffer, blots were developed with Clarity ECL substrates according to manufacturer directions on a Gel Doc™ XR+Imager with Image Lab software (BIO-RAD).

Gel images were analyzed using ImageJ/FIJI software for quantification. Calibration curves of known myc-tagged protein were used to establish a linear range (data not shown), and four points for each blot were included to allow absolute concentration determination. Three transfection replicates were included for each construct. For some constructs, the measured cellular level of protein was higher than the linear range of the calibration curve. However, for nearly all measurements, the secretion yield measurement was within linear range.

### Protein Biochemical Characterization

Dynamic light scattering measurements (DLS) were performed using the default Sizing and Polydispersity method on the UNcle™ (Unchained Labs). 8.8 μL of SEC-purified elution fractions were pipetted into the provided glass cuvettes. DLS measurements were run with ten replicates at 25° C. with an incubation time of 1 s; results were averaged across runs and plotted using Python. Other DLS measurements were also obtained using a DynaPro NanoStar™ (Wyatt) DLS setup with ten acquisitions per measurement, and three measurements per protein sample.

Samples were diluted to 0.1-0.02 mg/mL and 3 μL was negatively stained using Gilder Grids overlaid with a thin layer of carbon and 2% uranyl formate as previously described (Veesler et al. 2014). Data were collected on an Talos L120C 120 kV electron microscope equipped with a CETA camera.

To identify the molecular mass of each protein, intact mass spectra was obtained via reverse-phase LC/MS on an Agilent 6230B TOF on an AdvanceBio RP-Desalting column, and subsequently deconvoluted by way of Bioconfirm using a total entropy algorithm. For LC, buffers are water with 0.1% formic acid and acetonitrile with 0.1% formic acid; the proteins are eluted from a gradient of 10% to 100% in 2 min.

Except for KWOCA 39 (FIG. 4), for which the purification profile according to the described above is shown, SEC profiles for the described KWOCAS (FIG. 2) were obtained by high pressure liquid chromatography on an Agilent Bio SEC-5 column (Agilent) at a flow rate of 0.35 mL/min by injection of 10 μL of purified eluate ran in Tris-buffer saline (50 mM Tris pH 8, 150 mM NaCl, 5% v/v Glycerol).

### High-Resolution Structural Determination

Protein concentration was determined by A280 and using calculated molar extinction coefficients. Buffers, unless otherwise specified, are 50 mM Tris pH 8.0, 150 mM NaCl, and 5% glycerol.

Samples were prepared for small-angle X-ray scattering (SAXS) analysis after expression, purification, and size-exclusion chromatography as described above. Selected SEC fractions were concentrated to 1-5 mg/ml into buffer containing 2% glycerol. The flow-through was used as a blank for buffer subtraction during SAXS analysis. Samples were then centrifuged (13,000 g) and passed through a 0.22 μm syringe filter (Millipore). These proteins and buffer blanks were shipped to the SIBYLS™ High Throughput SAXS ALS Advanced Light Source in Berkeley, California to obtain scattering data (Putnam et al. 2007; Hura et al. 2009; Classen et al. 2013; Dyer et al. 2014). Scattering traces were analysed and fit to theoretical models using the FOXS™ 15 server (Schneidman-Duhovny et al. 2013, 2016).

All crystallization trials were carried out at 20° C. in 96-well format using the sitting-drop method. Crystal trays were set up using Mosquito LCP™ by SPT Labtech and monitored by JANSi UVEX imaging system. Drop volumes ranged from 200 to 400 nL and contained protein to crystallization solution in ratios of 1:1, 2:1, and 1:2. Diffraction quality crystals appeared in 0.2 M NH₄CH₃CO₂ 0.1 M Na₃C₆H₅O₇ pH 5.6 30% (v/v) MPD for KWOCA 39. Diffraction quality crystals appeared in 0.1 M Tris pH 7.0 20% (w/v) PEG-2000 MME for KWOCA 73. Diffraction quality crystals appeared in 0.2 M MgCl₂, 0.1 M Tris pH 7.0, 10% (w/v) PEG-8000 for KWOCA 60. Diffraction quality crystals appeared in 0.2 M MgCl₂, 0.1 M Imidazole pH 8.0, 35% (v/v) MPD for KWOCA 65. Diffraction quality crystals appeared in 0.1 M NaCH₄CO₂ pH 4.5, 35% (v/v) MPD for KWOCA 102. Crystals were subsequently harvested in a cryo-loop and flash frozen directly in liquid nitrogen for synchrotron data collection.

Data collection from crystals KWOCA 39, 60 and 65 were performed with synchrotron radiation at the Advanced Photon Source (APS) on 24ID-C. Data collection from crystals KWOCA 73, and 102 were performed with synchrotron radiation at the Advanced Light Source (ALS) on 8.2.1/8.2.2.

X-ray intensities and data reduction were evaluated and integrated using XDS (Kabsch 2010) and merged/scaled using Pointless/Aimless in the CCP4 program suite (Winn et al. 2011). Structure determination and refinement starting phases were obtained by molecular replacement using Phaser (McCoy et al. 2007) using the designed model for the structures. Following molecular replacement, the models were improved using phenix autobuild (Adams et al. 2010); efforts were made to reduce model bias by setting rebuild-in-place to false, and using simulated annealing and prime-and-switch phasing. Structures were refined in Phenix (Adams et al. 2010). Model building was performed using COOT (Emsley and Cowtan 2004). The final model was evaluated using MolProbity (Williams et al. 2018). Data collection and refinement statistics are recorded in Table 3.

TABLE 3
Crystallographic data collection and refinement statistics.

| | KWOCA_39 | | KWOCA_60 | | KWOCA_65 | | KWOCA_73 | | KWOCA_102 | |
|---|---|---|---|---|---|---|---|---|---|---|
| Data collection | | | | | | | | | | |
| Space group | H 3 | | H 3 | | P 63 2 2 | | P 2₁ | | P 4₃ 2₁ 2 | |
| Cell dimensions | | | | | | | | | | |
| a, b, c (Å) | 115.66, 115.66, 72.08 | | 90.53, 90.53, 125.52 | | 70.97, 70.97, 200.56 | | 72.00, 170.81, 72.15 | | 89.40, 89.40, 273.03 | |
| α, β, γ (°) | 90, 90, 120 | | 90, 90, 120 | | 90, 90, 120 | | 90, 117.83, 90 | | 90, 90, 90 | |
| Resolution (Å) | 58.51-3.61 | | 48.99-3.25 | | 61.27-3.15 | | 42.70-3.04 | | 50.0-3.80 | |
| | (3.96-3.61)* | | (3.51-3.25)* | | (3.37-3.15)* | | (3.11-3.04)* | | (3.87-3.8)* | |
| R_{merge} | 0.212 | (0.663) | 0.119 | (2.61) | 0.266 | (2.286) | 0.151 | (1.631) | 0.086 | (0.803) |
| I/σI | 4.2 | (1.8) | 10.2 | (5.5) | 6.0 | (1.1) | 4.8 | (0.8) | 22.6 | (2.7) |
| Completeness (%) | 100.0 | (100.0) | 100.0 | (100.0) | 100.0 | (100.0) | 99.2 | (99.9) | 100.0 | (100.0) |
| Unique reflections | 4127 | (983) | 52176 | (11095) | 65007 | (12073) | 29424 | (4770) | 11536 | (2668) |
| Redundancy | 5.4 | (5.6) | 8.6 | (8.6) | 11.3 | (12.1) | 3.7 | (3.8) | 16.5 | (15.6) |
| CC½ | 0.978 | (0.665) | 0.998 | (0.975) | 0.995 | (0.648) | 0.988 | (0.293) | 0.998 | (0.981) |
| Refinement | | | | | | | | | | |
| Resolution (Å) | 58.51-3.61 | | 41.63-3.25 | | 61.27-3.15 | | 42.70-3.04 | | 46.42-3.76 | |
| | (3.97-3.61) | | (4.09-3.25) | | (3.47-3.15)* | | (3.11-3.04) | | (3.85-3.76) | |
| No. reflections | 4126 | (1031) | 5938 | (2981) | 5660 | (1360) | 29378 | (2075) | 10455 | (1275) |

| | | | | | |
|---|---|---|---|---|---|
| R_{work}/R_{free} | 0.23/0.27 | 0.24/0.28 | 0.27/0.30 | 0.26/0.30 | 0.25/0.30 |
| | (0.36/0.38) | (0.24/0.30) | (0.31/0.38) | (0.39/0.43) | (0.32/0.40) |
| No. atoms | 2141 | 2386 | 1710 | 10153 | 5952 |
| Protein | 2141 | 2386 | 1710 | 10153 | 5952 |
| B-factors (Å²) | 183 | 29 | 96 | 109 | 78 |
| Protein | 183 | 29 | 96 | 109 | 78 |
| Bond lengths (Å) | 0.001 | 0.002 | 0.002 | 0.002 | 0.008 |
| Bond angles (°) | 0.326 | 1.336 | 0.347 | 0.371 | 1.309 |

*Data collected from a single crystal.
*Values in parentheses are for the highest-resolution shell.

For KWOCA 51, we applied 2 μL of 3 mg/mL of protein in 25 mM Tris, 150 mM NaCl, pH 8.0, 100 mM glycine to glow-discharged C-flat CF-2/2 C-T-grids (TED PELLA). KWOCA 51 data collection was performed on an FEI Titan Krios™ Electron Microscope operating at 300 kV. The microscope was equipped with a Gatan Quantum GIF energy filter and a K3 Summit direct electron detector (Li et al. 2013) operating in electron-counting mode. Nominal exposure magnification was 105,000 with the resulting pixel size at the 10 specimen plane of 0.85 A. Automated data collection was performed using Leginon software (Carragher et al. 2000; Suloway et al. 2005).

For KWOCA 51, all data processing was carried out in CryoSPARC. Alignment of movie frames was carried out using Patch Motion with an estimated B-factor of 500 Å². Defocus and astigmatism values were estimated using Patch CTF. ˜800,000 particles were picked in a reference-free manner using Blob Picker and extracted with a box size of 440 Å. An initial round of 2D classification was performed in CryoSPARC™ using 100 classes and a maximum alignment resolution of 6 A. ˜ 398,000 selected particles were re-centered and re-extracted with a box size of 360 Å. An ab initio reconstruction was generated using a Cl symmetry operator on the dose-weighted and re-centered particles. A homogeneous refinement was next performed using this ab initio model as a starting reference. Tetrahedral symmetry was applied during this refinement, leading to an initial estimated map resolution of 5.95 Å. Local motion within single movies was corrected using an estimated B-factor of 500 Å, and particles were re-extracted with a box size of 360 Å. A second round of homogeneous refinement was performed, resulting in an improved resolution estimate of 5.8 Å. Particles were next split into separate optics groups and re-refined to a final estimated resolution of 5.6 Å.

To calculate the root-mean-square (rms) deviation of the experimentally obtained crystal structures or relaxed cryo-EM model (experimental models) to the design model, the pair_fit function of PyMol was used on the common Ca carbons of the monomeric subunit of the pair of models to be compared. Additionally, the rms of the whole trimer was calculated using the rms_cur function on the common Ca carbons.

### Other Methods

Images for figures sourced from BioRender. Figures created using Inkscape. Data processing and plotting were performed with LibreOffice Calc and Python. Protein structure rendering was performed in PyMol or ChimeraX (Pettersen et al. 2021).

### REFERENCES

Abrescia, Nicola G. A., Dennis H. Bamford, Jonathan M. Grimes, and David I. Stuart. 2012. “Structure Unifies the Viral Universe.” Annual Review of Biochemistry 81 (April): 795-822.
Adams, Paul D., Pavel V. Afonine, Gábor Bunkóczi, Vincent B. Chen, Ian W. Davis, Nathaniel Echols, Jeffrey J. Headd, et al. 2010. “PHENIX: A Comprehensive Python-Based System for Macromolecular Structure Solution.” Acta Crystallographica. Section D, Biological Crystallography 66 (Pt 2): 213-21.
Akopian, David, Kuang Shen, Xin Zhang, and Shu-Ou Shan. 2013. “Signal Recognition Particle: An Essential Protein-Targeting Machine.” Annual Review of Biochemistry 82 (February): 693-721.
Baek, Minkyung, and David Baker. 2022. “Deep Learning and Protein Structure Modeling.” Nature Methods 19 (1): 13-14.
Bale, Jacob B., Shane Gonen, Yuxi Liu, William Sheffler, Daniel Ellis, Chantz Thomas, Duilio Cascio, et al. 2016. “Accurate Design of Megadalton-Scale Two-Component Icosahedral Protein Complexes.” Science 353 (6297): 389-94.
Barouch, Dan H., Zhi-Yong Yang, Wing-Pai Kong, Birgit Korioth-Schmitz, Shawn M. Sumida, Diana M. Truitt, Michael G. Kishko, et al. 2005. “A Human T-Cell Leukemia Virus Type 1 Regulatory Element Enhances the Immunogenicity of Human Immunodeficiency Virus Type 1 DNA Vaccines in Mice and Nonhuman Primates.” Journal of Virology 79 (14): 8828-34.
Baycin-Hizal, Deniz, David L. Tabb, Raghothama Chaerkady, Lily Chen, Nathan E. Lewis, Harish Nagarajan, Vishaldeep Sarkaria, et al. 2012. “Proteomic Analysis of Chinese Hamster Ovary Cells.” Journal of Proteome Research 11 (11): 5265-76.
Bobik, Thomas A., Brent P. Lehman, and Todd O. Yeates. 2015. “Bacterial Microcompartments: Widespread Prokaryotic Organelles for Isolation and Optimization of Metabolic Pathways.” Molecular Microbiology 98 (2): 193-207.
Boone, Morgane, Pathmanaban Ramasamy, Jasper Zuallaert, Robbin Bouwmeester, Berre Van Moer, Davy Maddelein, Demet Turan, et al. 2021. “Massively Parallel Interrogation of Protein Fragment Secretability Using SECRIFY Reveals Features Influencing Secretory System Transit.” Nature Communications 12 (1): 6414.
Boyken, Scott E., Mark A. Benhaim, Florian Busch, Mengxuan Jia, Matthew J. Bick, Heejun Choi, Jason C. Klima, et al. 2019. “De Novo Design of Tunable, pH-Driven Conformational Changes.” Science 364 (6441): 658-64.
Boyoglu-Barnum, Seyhan, Daniel Ellis, Rebecca A. Gillespie, Geoffrey B. Hutchinson, Young-Jun Park, Syed M. Moin, Oliver J. Acton, et al. 2021. “Quadrivalent Influenza Nanoparticle Vaccines Induce Broad Protection.” Nature 592 (7855): 623-28.
Braun, Elisabeth, and Daniel Sauter. 2019. “Furin-Mediated Protein Processing in Infectious Diseases and Cancer.” Clinical & Translational Immunology 8 (8): e1073.
Brunette, T. J., Fabio Parmeggiani, Po-Ssu Huang, Gira Bhabha, Damian C. Ekiert, Susan E. Tsutakawa, Greg L. Hura, John A. Tainer, and David Baker. 2015. “Exploring the Repeat Protein Universe through Computational Protein Design.” Nature 528 (7583): 580-84.
Bruun, Theodora U. J., Anne-Marie C. Andersson, Simon J. Draper, and Mark Howarth. 2018. “Engineering a Rugged Nanoscaffold To Enhance Plug-and-Display Vaccination.” ACS Nano 12 (9): 8855-66.
Burley, Stephen K., Charmi Bhikadiya, Chunxiao Bi, Sebastian Bittrich, Li Chen, Gregg V. Crichlow, Cole H. Christie, et al. 2021. “RCSB Protein Data Bank: Powerful New Tools for Exploring 3D Structures of Biological Macromolecules for Basic and Applied Research and Education in Fundamental Biology, Biomedicine, Biotechnology, Bioengineering and Energy Sciences.” Nucleic Acids Research 49 (D1): D437-51.
Campeotto, Ivan, Adi Goldenzweig, Jack Davey, Lea Barfod, Jennifer M. Marshall, Sarah E. Silk, Katherine E. Wright, Simon J. Draper, Matthew K. Higgins, and Sarel J. Fleishman. 2017. “One-Step Design of a Stable Variant of the Malaria Invasion Protein RH5 for Use as a Vaccine Immunogen.” Proceedings of the National Academy of Sciences of the United States of America 114 (5): 998-1002
Carragher, B., N. Kisseberth, D. Kriegman, R. A. Milligan, C. S. Potter, J. Pulokas, and A. Reilein. 2000. “Leginon: An Automated System for Acquisition of Images from Vitreous Ice Specimens.” Journal of Structural Biology 132 (1): 33-45.
Chaudhary, Namit, Drew Weissman, and Kathryn A. Whitehead. 2021. “mRNA Vaccines for Infectious Diseases: Principles, Delivery and Clinical Translation.” Nature Reviews. Drug Discovery 20 (11): 817-38.
Classen, Scott, Greg L. Hura, James M. Holton, Robert P. Rambo, Ivan Rodic, Patrick J. McGuire, Kevin Dyer, et al. 2013. “Implementation and Performance of SIBYLS: A Dual Endstation Small-Angle X-Ray Scattering and Macromolecular Crystallography Beamline at the Advanced Light Source.” Journal of Applied Crystallography 46 (Pt 1): 1-13.
Cohen, Alexander A., Priyanthi N. P. Gnanapragasam, Yu E. Lee, Pauline R. Hoffman, Susan Ou, Leesa M. Kakutani, Jennifer R. Keeffe, et al. 2021. “Mosaic Nanoparticles Elicit Cross-Reactive Immune Responses to Zoonotic Coronaviruses in Mice.” Science 371 (6530): 735-41.
Dalvie, Neil C., Joseph R. Brady, Laura E. Crowell, Mary Kate Tracey, Andrew M. Biedermann, Kawaljit Kaur, John M. Hickey, et al. 2021, “Molecular Engineering Improves Antigen Quality and Enables Integrated Manufacturing of a Trivalent Subunit Vaccine Candidate for Rotavirus.” Microbial Cell Factories 20 (1): 94.
Dalvie, Neil C., Sergio A. Rodriguez-Aponte, Brittany L. Hartwell, Lisa H. Tostanoski, Andrew M. Biedermann, Laura E. Crowell, Kawaljit Kaur, et al. 2021. “Engineered SARS-COV-2 Receptor Binding Domain Improves Manufacturability in Yeast and Immunogenicity in Mice.” Proceedings of the National Academy of Sciences of the United States of America 118 (38). doi.org/10.1073/pnas.2106845118.
DiMaio, Frank, Andrew Leaver-Fay, Phil Bradley, David Baker, and Ingemar André. 2011. “Modeling Symmetric Macromolecular Structures in Rosetta3.” PloS One 6 (6): €20450.
DiMaio, Frank, Yifan Song, Xueming Li, Matthias J. Brunner, Chunfu Xu, Vincent Conticello, Edward Egelman, Thomas Marlovits, Yifan Cheng, and David Baker. 2015. “Atomic-Accuracy Models from 4.5-A Cryo-Electron Microscopy Data with Density-Guided Iterative Local Refinement.” Nature Methods 12 (4): 361-65.
Douglas, Trevor, and Mark Young. 2006. “Viruses: Making Friends with Old Foes.” Science 312 (5775): 873-75.
Dou, Jiayi, Anastassia A. Vorobieva, William Sheffler, Lindsey A. Doyle, Hahnbeom Park, Matthew J. Bick, Binchen Mao, et al. 2018. “De Novo Design of a Fluorescence-Activating β-Barrel.” Nature 561 (7724): 485-91.
Dyer, Kevin N., Michal Hammel, Robert P. Rambo, Susan E. Tsutakawa, Ivan Rodic, Scott Classen, John A. Tainer, and Greg L. Hura. 2014. “High-Throughput SAXS for the Characterization of Biomolecules in Solution: A Practical Approach.” Methods in Molecular Biology 1091:245-58.
Edwardson, Thomas G. W., Stephan Tetter, and Donald Hilvert. 2020. “Two-Tier Supramolecular Encapsulation of Small Molecules in a Protein Cage.” Nature Communications 11 (1): 5410.
Egea, Pascal F., Robert M. Stroud, and Peter Walter, 2005. “Targeting Proteins to Membranes: Structure of the Signal Recognition Particle.” Current Opinion in Structural Biology 15 (2): 213-20.
Ellis, Daniel, Natalie Brunette, Katharine H. D. Crawford, Alexandra C. Walls, Minh N. Pham, Chengbo Chen, Karla-Luise Herpoldt, et al. 2021. “Stabilization of the SARS-CoV-2 Spike Receptor-Binding Domain Using Deep Mutational Scanning and Structure-Based Design.” Frontiers in Immunology 12 (June): 710263.
Emsley, Paul, and Kevin Cowtan. 2004, “Coot: Model-Building Tools for Molecular Graphics.” Acta Crystallographica. Section D, Biological Crystallography 60 (Pt 12 Pt 1): 2126-32.
Fallas, Jorge A., George Ueda, William Sheffler, Vanessa Nguyen, Dan E. McNamara, Banumathi Sankaran, Jose Henrique Pereira, et al. 2017. “Computational Design of Self-Assembling Cyclic Protein Homo-Oligomers.” Nature Chemistry 9 (4): 353-60.
Farhan, Hesso, and Catherine Rabouille. 2011. “Signalling to and from the Secretory Pathway.” Journal of Cell Science 124 (Pt 2): 171-80.
Goodsell, D. S., and A. J. Olson. 2000. “Structural Symmetry and Protein Function.” Annual Review of Biophysics and Biomolecular Structure 29:105-53.
Gordon, Sydney R., Elizabeth J. Stanley, Sarah Wolf, Angus Toland, Sean J. Wu, Daniel Hadidi, Jeremy H. Mills, David Baker, Ingrid Swanson Pultz, and Justin B. Siegel. 2012. “Computational Design of an α-Gliadin Peptidase.” Journal of the American Chemical Society 134 (50): 20513-20.
Güler-Gane, Gülin, Sara Kidd, Sudharsan Sridharan, Tristan J. Vaughan, Trevor C. I. Wilkinson, and Natalie J. Tigue. 2016. “Overcoming the Refractory Expression of Secreted Recombinant Proteins in Mammalian Cells through Modification of the Signal Peptide and Adjacent Amino Acids.” PloS One 11 (5): e0155340.
Heijne, G. von. 1990. “The Signal Peptide.” The Journal of Membrane Biology 115 (3): 195-201
He, Linling, Xiaohe Lin, Ying Wang, Ciril Abraham, Cindy Sou, Timothy Ngo, Yi Zhang, Ian A. Wilson, and Jiang Zhu. 2021. “Single-Component, Self-Assembling, Protein Nanoparticles Presenting the Receptor Binding Domain and Stabilized Spike as SARS-CoV-2 Vaccine Candidates.” Science Advances 7 (12): cabf1591.
Hessa, Tara, Hyun Kim, Karl Bihlmaier, Carolina Lundin, Jorrit Boekel, Helena Andersson, Ingmarie Nilsson, Stephen H. White, and Gunnar von Heijne. 2005. “Recognition of Transmembrane Helices by the Endoplasmic Reticulum Translocon.” Nature 433 (7024): 377-81.
Hessa, Tara, Nadja M. Meindl-Beinker, Andreas Bernsel, Hyun Kim, Yoko Sato, Mirjam Lerch-Bader, Ingmarie Nilsson, Stephen H. White, and Gunnar von Heijne. 2007. “Molecular Code for Transmembrane-Helix Recognition by the Sec61 Translocon,” Nature 450 (7172): 1026-30.
Hsia, Yang, Jacob B. Bale, Shane Gonen, Dan Shi, William Sheffler, Kimberly K. Fong, Una Nattermann, et al. 2016. “Design of a Hyperstable 60-Subunit Protein Icosahedron.” Nature 535 (7610): 136-39.
Hsia, Yang, Rubul Mout, William Sheffler, Natasha I. Edman, Ivan Vulovic, Young-Jun Park, Rachel L. Redler, et al. 2021. “Design of Multi-Scale Protein Complexes by Hierarchical Building Block Fusion.” Nature Communications 12 (1): 2294.
Hsieh, Ching-Lin, Jory A. Goldsmith, Jeffrey M. Schaub, Andrea M. DiVenere, Hung-Che Kuo, Kamyab Javanmardi, Kevin C. Le, et al. 2020. “Structure-Based Design of Prefusion-Stabilized SARS-COV-2 Spikes.” Science 369 (6510): 1501-5.
Huang, Po-Ssu, Scott E. Boyken, and David Baker. 2016. “The Coming of Age of de Novo Protein Design.” Nature 537 (7620): 320-27.
Hura, Greg L., Angeli L. Menon, Michal Hammel, Robert P. Rambo, Farris L. Poole 2nd, Susan E. Tsutakawa, Francis E. Jenney Jr, et al. 2009. “Robust, High-Throughput Solution Structural Analyses by Small Angle X-Ray Scattering (SAXS).” Nature Methods 6 (8): 606-12.
Janin, Joël, Ranjit P. Bahadur, and Pinak Chakrabarti. 2008. “Protein-Protein Interaction and Quaternary Structure.” Quarterly Reviews of Biophysics 41 (2): 133-80.
Jardine, Joseph, Jean-Philippe Julien, Sergey Menis, Takayuki Ota, Oleksandr Kalyuzhniy, Andrew McGuire, Devin Sok, et al. 2013. “Rational HIV Immunogen Design to Target Specific Germline B Cell Receptors.” Science 340 (6133): 711-16.
Kabsch, Wolfgang. 2010. “XDS.” Acta Crystallographica. Section D, Biological Crystallography 66 (Pt 2): 125-32.
Kanekiyo, Masaru, Chih-Jen Wei, Hadi M. Yassine, Patrick M. McTamney, Jeffrey C. Boyington, James R. R. Whittle, Srinivas S. Rao, Wing-Pui Kong, Lingshu Wang, and Gary J. Nabel. 2013. “Self-Assembling Influenza Nanoparticle Vaccines Elicit Broadly Neutralizing HIN1 Antibodies.” Nature 499 (7456): 102-6.
Kelley, Brian. 2009i. “Industrialization of mAb Production Technology: The Bioprocessing Industry at a Crossroads.” mAbs 1 (5): 443-52.
Khmelinskaia, Alena, Adam Wargacki, and Neil P. King. 2021. “Structure-Based Design of Novel Polyhedral Protein Nanomaterials.” Current Opinion in Microbiology 61 (June): 51-57.
King, Neil P., Jacob B. Bale, William Sheffler, Dan E. McNamara, Shane Gonen, Tamir Gonen, Todd O. Yeates, and David Baker. 2014. “Accurate Design of Co-Assembling Multi-Component Protein Nanomaterials.” Nature 510 (7503): 103-8.
King, Neil P., William Sheffler, Michael R. Sawaya, Breanna S. Vollmar, John P. Sumida, Ingemar André, Tamir Gonen, Todd O. Yeates, and David Baker. 2012. “Computational Design of Self-Assembling Protein Nanomaterials with Atomic Level Accuracy.” Science 336 (6085): 1171-74.
Klima, Jason C., Lindsey A. Doyle, Justin Daho Lee, Michael Rappleye, Lauren A. Gagnon, Min Yen Lee, Emilia P. Barros, et al. 2021. “Incorporation of Sensing Modalities into de Novo Designed Fluorescence-Activating Proteins.” Nature Communications 12 (1): 856.
Kober, Lars, Christoph Zehe, and Juergen Bode. 2013. “Optimized Signal Peptides for the Development of High Expressing CHO Cell Lines,” Biotechnology and Bioengineering 110 (4): 1164-73.
Konrath, Kylie M., Kevin Liaw, Yuanhan Wu, Xizhou Zhu, Susanne N. Walker, Ziyang Xu, Katherine Schultheis, et al. 2022. “Nucleic Acid Delivery of Immune-Focused SARS-CoV-2 Nanoparticles Drives Rapid and Potent Immunogenicity Capable of Single-Dose Protection.” Cell Reports, January, 110318.
Kuo, Chih-Chung, Austin Wt Chiang, Isaac Shamie, Mojtaba Samoudi, Jahir M. Gutierrez, and Nathan E. Lewis. 2018. “The Emerging Role of Systems Biology for Engineering Protein Production in CHO Cells.” Current Opinion in Biotechnology 51 (June): 64-69.
Lawrence, M. C., and P. M. Colman. 1993. “Shape Complementarity at Protein/protein Interfaces.” Journal of Molecular Biology 234 (4): 946-50.
Leaver-Fay, Andrew, Michael Tyka, Steven M. Lewis, Oliver F. Lange, James Thompson, Ron Jacak, Kristian Kaufman, et al. 2011. “ROSETTA3: An Object-Oriented Software Suite for the Simulation and Design of Macromolecules.” Methods in Enzymology 487:545-74.
Le Fourn, Valérie, Pierre-Alain Girod, Montse Buceta, Alexandre Regamey, and Nicolas Mermod. 2014. “CHO Cell Engineering to Prevent Polypeptide Aggregation and Improve Therapeutic Protein Secretion.” Metabolic Engineering 21 (January): 91-102.
Leman, Julia Kochler, Brian D. Weitzner, Steven M. Lewis, Jared Adolf-Bryfogle, Nawsad Alam, Rebecca F. Alford, Melanie Aprahamian, et al. 2020. “Macromolecular Modeling and Design in Rosetta: Recent Methods and Frameworks.” Nature Methods 17 (7): 665-80.
Liu, Yan, Anton Nguyen, Robert L. Wolfert, and Shaoqiu Zhuo. 2009. “Enhancing the Secretion of Recombinant Proteins by Engineering N-Glycosylation Sites.” Biotechnology Progress 25 (5): 1468-75.
Li, Xueming, Paul Mooney, Shawn Zheng, Christopher R. Booth, Michael B. Braunfeld, Sander Gubbens, David A. Agard, and Yifan Cheng. 2013. “Electron Counting and Beam-Induced Motion Correction Enable near-Atomic-Resolution Single-Particle Cryo-EM.” Nature Methods 10 (6): 584-90.
López-Sagaseta, Jacinto, Enrico Malito, Rino Rappuoli, and Matthew J. Bottomley. 2016. “Self-Assembling Protein Nanoparticles in the Design of Vaccines.” Computational and Structural Biotechnology Journal 14:58-68.
Malladi, Sameer Kumar, David Schreiber, Ishika Pramanick, Malavika Abhineshababu Sridevi, Adi Goldenzweig, Somnath Dutta, Sarel Jacob Fleishman, and Raghavan Varadarajan. 2020. “One-Step Sequence and Structure-Guided Optimization of HIV-1 Envelope gp140i.” Current Research in Structural Biology 2 (April): 45-55.
Marcandalli, Jessica, Brooke Fiala, Sebastian Ols, Michela Perotti, Willem de van der Schueren, Joost Snijder, Edgar Hodge, et al. 2019, “Induction of Potent Neutralizing Antibody Responses by a Designed Protein Nanoparticle Vaccine for Respiratory Syncytial Virus.” Cell 176 (6): 1420-31.e17.
McCoy, Airlie J., Ralf W. Grosse-Kunstleve, Paul D. Adams, Martyn D. Winn, Laurent C. Storoni, and Randy J. Read. 2007. “Phaser Crystallographic Software.” Journal of Applied Crystallography 40 (Pt 4): 658-74.
Mu, Zekun, Kevin Wiche, Kevin O. Saunders, Rory Henderson, Derek W. Cain, Robert Parks, Diana Martik, et al. 2021i. “Ability of Nucleoside-Modified mRNA to Encode HIV-1 Envelope Trimer Nanoparticles.” bioRxiv: The Preprint Server for Biology, August. doi.org/10.1101/2021.08.09.455714.
Ohtsubo, Kazuaki, and Jamey D. Marth. 2006. “Glycosylation in Cellular Mechanisms of Health and Disease.” Cell 126 (5): 855-67.
Padilla, J. E., C. Colovos, and T. O. Yeates. 2001. “Nanohedra: Using Symmetry to Design Self Assembling Protein Cages, Layers, Crystals, and Filaments.” Proceedings of the National Academy of Sciences of the United States of America 98 (5): 2217-21.
Pallesen, Jesper, Nianshuang Wang, Kizzmekia S. Corbett, Daniel Wrapp, Robert N. Kirchdoerfer, Hannah L. Turner, Christopher A. Cottrell, et al. 2017. “Immunogenicity and Structures of a Rationally Designed Prefusion MERS-COV Spike Antigen.” Proceedings of the National Academy of Sciences of the United States of America 114 (35): E7348-57.
Palombarini, Federica, Elisa Di Fabio, Alberto Boffi, Alberto Macone, and Alessandra Bonamore. 2020. “Ferritin Nanocages for Protein Delivery to Tumor Cells.” Molecules 25 (4). doi.org/10.3390/molecules25040825.
Pardi, Norbert, Michael J. Hogan, Frederick W. Porter, and Drew Weissman. 2018. “mRNA Vaccines—a New Era in Vaccinology.” Nature Reviews. Drug Discovery 17 (4): 261-79.
Peleg, Yoav, Renaud Vincentelli, Brett M. Collins, Kai-En Chen, Emma K. Livingstone, Saroja Weeratunga, Natalya Leneva, et al. 2021. “Community-Wide Experimental Evaluation of the PROSS Stability-Design Method.” Journal of Molecular Biology 433 (13): 166964.
Pettersen, Eric F., Thomas D. Goddard, Conrad C. Huang, Gregory S. Couch, Daniel M. Greenblatt, Elaine C. Meng, and Thomas E. Ferrin. 2004. “UCSF Chimera—a Visualization System for Exploratory Research and Analysis.” Journal of Computational Chemistry 25 (13): 1605-12.
Pettersen, Eric F., Thomas D. Goddard, Conrad C. Huang, Elaine C. Meng, Gregory S. Couch, Tristan I. Croll, John H. Morris, and Thomas E. Ferrin. 2021. “UCSF ChimeraX: Structure Visualization for Researchers, Educators, and Developers.” Protein Science: A Publication of the Protein Society 30 (1): 70-82.
Punjani, Ali, John L. Rubinstein, David J. Fleet, and Marcus A. Brubaker. 2017. “cryoSPARC: Algorithms for Rapid Unsupervised Cryo-EM Structure Determination.” Nature Methods 14 (3): 290-96.
Putnam, Christopher D., Michal Hammel, Greg L. Hura, and John A. Tainer. 2007. “X-Ray Solution Scattering (SAXS) Combined with Crystallography and Computation: Defining Accurate Macromolecular Structures, Conformations and Assemblies in Solution.” Quarterly Reviews of Biophysics 40 (3): 191-285.
Rajoo, Sasikumar, Pascal Vallotton, Evgeny Onischenko, and Karsten Weis. 2018. “Stoichiometry and Compositional Plasticity of the Yeast Nuclear Pore Complex Revealed by Quantitative Fluorescence Microscopy.” Proceedings of the National Academy of Sciences of the United States of America 115 (17): E3969-77.
Rakestraw, J. Andy, Stephen L. Sazinsky, Andrea Piatesi, Eugene Antipov, and K. Dane Wittrup. 2009. “Directed Evolution of a Secretory Leader for the Improved Expression of Heterologous Proteins and Full-Length Antibodies in Saccharomyces Cerevisiae.” Biotechnology and Bioengineering 103 (6): 1192-1201.
Rapoport, Tom A. 2007. “Protein Translocation across the Eukaryotic Endoplasmic Reticulum and Bacterial Plasma Membranes.” Nature 450 (7170): 663-69.
Rawi, Reda, Lucy Rutten, Yen-Ting Lai, Adam S. Olia, Sven Blokland, Jarek Juraszek, Chen-Hsiang Shen, et al. 2020. “Automated Design by Structure-Based Stabilization and Consensus Repair to Achieve Prefusion-Closed Envelope Trimers in a Wide Variety of HIV Strains.” Cell Reports 33 (8): 108432.
Rojas, Gertrudis, Tania Carmenate, Julio Felipe Santo-Tomás, Pedro A. Valiente, Marlies Becker, Annia Pérez-Riverón, Yaima Tundidor, et al. 2019. “Directed Evolution of Super-Secreted Variants from Phage-Displayed Human Interleukin-2i.” Scientific Reports 9 (1): 800.
Ruiter, M. V. de, R. M. van der Hee, A. J. M. Driessen, E. D. Keurhorst, M. Hamid, and J. J. L. M. Cornelissen. 2019. “Polymorphic Assembly of Virus-Capsid Proteins around DNA and the Cellular Uptake of the Resulting Particles.” Journal of Controlled Release; Official Journal of the Controlled Release Society 307 (August): 342-54.
Rupp, Oliver, Madolyn L. MacDonald, Shangzhong Li, Heena Dhiman, Shawn Polson, Sven Griep, Kelley Heffner, et al. 2018. “A Reference Genome of the Chinese Hamster Based on a Hybrid Assembly Strategy.” Biotechnology and Bioengineering 115 (8): 2087-2100
Sagt, C. M., B. Kleizen, R. Verwaal, M. D. de Jong, W. H. Müller, A. Smits, C. Visser, J. Boonstra, A. J. Verkleij, and C. T. Verrips. 2000. “Introduction of an N-Glycosylation Site Increases Secretion of Heterologous Proteins in Yeasts.” Applied and Environmental Microbiology 66 (11): 4940-44.
Sahasrabuddhe, Aniruddha, Yang Hsia, Florian Busch, William Sheffler, Neil P. King, David Baker, and Vicki H. Wysocki. 2018. “Confirmation of Intersubunit Connectivity and Topology of Designed Protein Complexes by Native MS.” Proceedings of the National Academy of Sciences of the United States of America 115 (6): 1268-73.
Sahtoe, Danny D., Florian Praetorius, Alexis Courbet, Yang Hsia, Basile I. M. Wicky, Natasha I. Edman, Lauren M. Miller, et al. 2022. “Reconfigurable Asymmetric Protein Assemblies through Implicit Negative Design.” Science 375 (6578): cabj7662.
Schneidman-Duhovny, Dina, Michal Hammel, John A. Tainer, and Andrej Sali. 2013. “Accurate SAXS Profile Computation and Its Assessment by Contrast Variation Experiments.” Biophysical Journal 105 (4): 962-74. 2016. “FoXS, FoXSDock and MultiFoXS: Single-State and Multi-State Structural Modeling of Proteins and Their Complexes Based on SAXS Profiles.” Nucleic Acids Research 44 (W1): W424-29.
Silva, Daniel-Adriano, Shawn Yu, Umut Y. Ulge, Jamie B. Spangler, Kevin M. Jude, Carlos Labão-Almeida, Lestat R. Ali, et al. 2019. “De Novo Design of Potent and Selective Mimics of IL-2 and IL-15i.” Nature 565 (7738): 186-91.
Suloway, Christian, James Pulokas, Denis Fellmann, Anchi Cheng, Francisco Guerra, Joel Quispe, Scott Stagg, Clinton S. Potter, and Bridget Carragher. 2005. “Automated Molecular Microscopy: The New Leginon System.” Journal of Structural Biology 151 (1): 41-60.
Tetter, Stephan, Naohiro Terasaka, Angela Steinauer, Richard J. Bingham, Sam Clark, Andrew J. P. Scott, Nikesh Patel, et al. 2021. “Evolution of a Virus-like Architecture and Packaging Mechanism in a Repurposed Bacterial Protein.” Science 372 (6547): 1220-24.
Ueda, George, Aleksandar Antanasijevic, Jorge A. Fallas, William Sheffler, Jeffrey Copps, Daniel Ellis, Geoffrey B. Hutchinson, et al. 2020a. “Tailored Design of Protein Nanoparticle Scaffolds for Multivalent Presentation of Viral Glycoprotein Antigens.” eLife 9 (August). doi.org/10.7554/eLife.57659. 2020b. “Tailored Design of Protein Nanoparticle Scaffolds for Multivalent Presentation of Viral Glycoprotein Antigens.” eLife 9 (August). doi.org/10.7554/eLife.57659.
Uhlen, Mathias, Max J. Karlsson, Andreas Hober, Anne-Sophie Svensson, Julia Scheffel, David Kotol, Wen Zhong, et al. 2019. “The Human Secretome.” Science Signaling 12 (609): caaz0274.
UniProt Consortium. 2021i. “UniProt: The Universal Protein Knowledgebase in 2021i.” Nucleic Acids Research 49 (DI): D480-89.
Veesler, David, Karolina Cupelli, Markus Burger, Peter Gräber, Thilo Stehle, and John E. Johnson. 2014. “Single-Particle EM Reveals Plasticity of Interactions between the Adenovirus Penton Base and Integrin aVB3i.” Proceedings of the National Academy of Sciences of the United States of America 111 (24): 8815-19.
Vieira Gomes, Antonio Milton, Talita Souza Carmo, Lucas Silva Carvalho, Frederico Mendonça Bahia, and Nádia Skorupa Parachin. 2018. “Comparison of Yeasts as Hosts for Recombinant Protein Production.” Microorganisms 6 (2). doi.org/10.3390/microorganisms6020038.
Walls, Alexandra C., Brooke Fiala, Alexandra Schäfer, Samuel Wrenn, Minh N. Pham, Michael Murphy, Longping V. Tse, et al. 2020. “Elicitation of Potent Neutralizing Antibody Responses by Designed Protein Nanoparticle Vaccines for SARS-COV-2i.” Cell 183 (5): 1367-82.e17.
Walsh, Gary. 2018i. “Biopharmaceutical Benchmarks 2018.” Nature Biotechnology 36 (12): 1136-45.
Wang, Dan, Phillip W. L. Tai, and Guangping Gao. 2019. “Adeno-Associated Virus Vector as a Platform for Gene Therapy Delivery.” Nature Reviews. Drug Discovery 18 (5): 358-78.
Wang, Ray Yu-Ruei, Yifan Song, Benjamin A. Barad, Yifan Cheng, James S. Fraser, and Frank DiMaio. 2016. “Automated Structure Refinement of Macromolecular Assemblies from Cryo-EM Maps Using Rosetta.” eLife 5 (September). doi.org/10.7554/eLife.17219.
Williams, Christopher J., Jeffrey J. Headd, Nigel W. Moriarty, Michael G. Prisant, Lizbeth L. Videau, Lindsay N. Deis, Vishal Verma, et al. 2018. “MolProbity: More and Better Reference Data for Improved All-Atom Structure Validation,” Protein Science: A Publication of the Protein Society 27 (1): 293-315.
Winn, Martyn D., Charles C. Ballard, Kevin D. Cowtan, Eleanor J. Dodson, Paul Emsley, Phil R. Evans, Ronan M. Keegan, et al. 2011. “Overview of the CCP4 Suite and Current Developments.” Acta Crystallographica. Section D, Biological Crystallography 67 (Pt 4): 235-42.
Wittrup, K. D. 1995. “Disulfide Bond Formation and Eukaryotic Secretory Productivity.” Current Opinion in Biotechnology 6 (2): 203-8.
Xiao, Su, Joseph Shiloach, and Michael J. Betenbaugh. 2014. “Engineering Cells to Improve Protein Expression.” Current Opinion in Structural Biology 26 (June): 32-38.
Xu, Xun, Harish Nagarajan, Nathan E. Lewis, Shengkai Pan, Zhiming Cai, Xin Liu, Wenbin Chen, et al. 2011. “The Genomic Sequence of the Chinese Hamster Ovary (CHO)-KI Cell Line.” Nature Biotechnology 29 (8): 735-41.
Xu, Ziyang, Megan C. Wise, Neethu Chokkalingam, Susanne Walker, Edgar Tello-Ruiz, Sarah T. C. Elliott, Alfredo Perales-Puchalt, et al. 2020. “In Vivo Assembly of Nanoparticles Achieved through Synergy of Structure-Based Protein Engineering and Synthetic DNA Generates Enhanced Adaptive Immunity.” Advancement of Science 7 (8): 1902802.
Zhang, Kai, 2016. “Getf: Real-Time CTF Determination and Correction.” Journal of Structural Biology 193 (1): 1-12.
Zhang, Shuguang. 2003. “Fabrication of Novel Biomaterials through Molecular Self-Assembly.” Nature Biotechnology 21 (10): 1171-78.
Zheng, Shawn Q., Eugene Palovcak, Jean-Paul Armache, Kliment A. Verba, Yifan Cheng, and David A. Agard. 2017. “MotionCor2: Anisotropic Correction of Beam-Induced Motion for Improved Cryo-Electron Microscopy.” Nature Methods 14 (4): 331-32.
Zivanov, Jasenko, Takanori Nakane, Björn O. Forsberg, Dari Kimanius, Wim Jh Hagen, Erik Lindahl, and Sjors Hw Scheres. 2018. “New Tools for Automated High-Resolution Cryo-EM Structure Determination in RELION-3i.” eLife 7 (November). doi.org/10.7554/eLife.42166.

### Example 2

### Characterization of Antigen-Bearing Secretion-Optimized Protein Nanoparticles

Given the success of the Degreaser in designing robustly secreting protein nanoparticles, we next used these nanoparticles (also referred to as KWOCAs) as scaffolds to display multiple copies of wild-type (WT) and stabilized (Rpk9) monomers of the severe acute respiratory syndrome coronavirus 2 (SARS-COV-2) receptor binding domain (RBD). Only KWOCAs that were verified by negative-stain electron microscopy (nsEM) and had preferred termini orientation (i.e., facing toward the outside of the nanoparticle) were considered for antigen display; the only exception being KWOCA 96, which had not been nsEM verified. For KWOCAs 4, 18, 46, 51, 96, 100, and 101, RBD monomers were genetically fused to the outward facing N termini of the nanoparticle subunits. For KWOCAs 47 and 70, RBD monomers were genetically fused to the outward facing C termini of the nanoparticle subunits. Additionally, a previously designed, retroactively degreased nanoparticle called 13-01-NS had RBD monomers genetically fused to the outward facing N termini of the nanoparticle subunits.

To retain glycosylation patterns and disulfide bonds in the RBD, we only expressed the antigen-bearing nanoparticles via transient transfection of Expi293F cells. We first expressed at a small scale (1 mL cultures) to determine which constructs were antigenically intact. During biolayer interferometry (BLI), 8 of the 10 supernatants bound the RBD-specific CV30 antibody tighter than purified monomeric RBD, indicating that those antigen-bearing nanoparticles were antigenically intact. The 2 supernatants that did not bind CV30 were the constructs where RBD monomers were genetically fused to the C termini of the nanoparticle. Of the 8 antigenically intact antigen-bearing nanoparticles, 5 were arbitrarily chosen to be expressed at a large scale (200 mL cultures) for further biochemical and biophysical characterization.

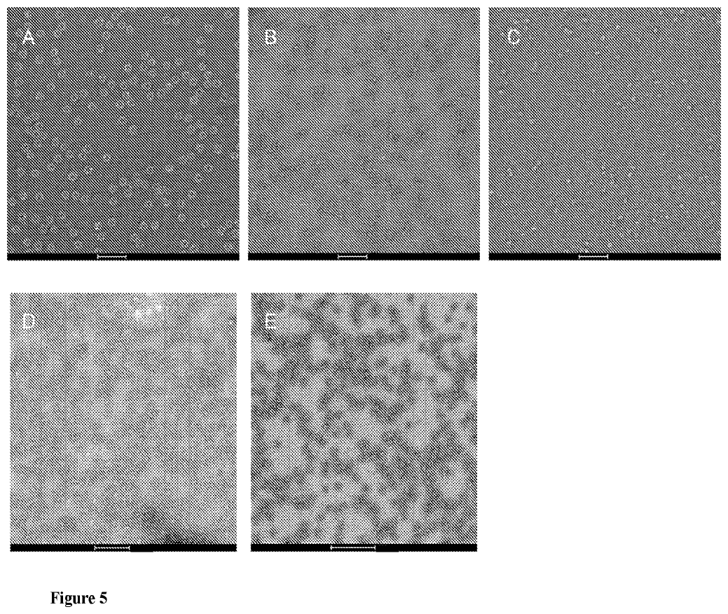

During size exclusion chromatography (SEC) purification, Rpk9_RBD_SARS-COV-2_13-01-NS, Rpk9_RBD_SARS-COV-2_KWOCA-51, and Rpk9_RBD_SARS-COV-2_KWOCA-101 yielded single peaks with elution volumes (˜10, 14, and 14 mL, respectively) corresponding to protein complexes (13, T3, and T3, respectively) of the expected molecular weights. Dynamic light scattering (DLS) of fractions from these peaks indicated the formation of monodisperse assemblies with expected hydrodynamic diameters (˜48, 21, and 21 nm, respectively). Further, nsEM confirmed the assembly of homogenous antigen-bearing nanoparticles (FIG. 5A-C). Additionally, to confirm that large scale expression and purification had not negatively impacted antigenicity, we used BLI to analyze binding of the final purified antigen-bearing nanoparticles. Rpk9_RBD_SARS-COV-2 13-01-NS, Rpk9_RBD_SARS-COV-2_KWOCA-51, and Rpk9_RBD_SARS-COV-2 KWOCA-101 bound CR3022 more tightly than purified monomeric RBD, indicating that the antigen-bearing nanoparticles remained antigenically intact.

During SEC purification, Rpk9_RBD_SARS-COV-2_KWOCA-18 yielded two peaks, one minor and one major. The elution volume of the minor peak (˜10 mL) corresponded to a protein complex with higher molecular weight than expected, but slightly lower than that of an unbounded aggregate. The elution volume of the major peak (˜13 mL) corresponded to a protein complex (D5) of the expected molecular weight. DLS of SEC fractions from each peak indicated the formation of aggregates in the minor peak and monodisperse assemblies with expected hydrodynamic diameters (˜ 33 nm) in the major peak. Further, nsEM of combined major peak fractions confirmed the assembly of homogenous antigen-bearing nanoparticles (FIG. 5D). Additionally, to confirm that large scale expression and purification bad not negatively impacted antigenicity, we used BLI to analyze binding of the final purified antigen-bearing nanoparticles. Rpk9_RBD_SARS-COV-2 KWOCA-18 bound CR3022 more tightly than purified monomeric RBD, indicating that the antigen-bearing nanoparticles remained antigenically intact.

During SEC purification, Rpk9_RBD_SARS-COV-2_KWOCA-4 yielded three peaks, two minor and one major. The elution volume of the first minor peak (˜10 mL) corresponded to a protein complex with higher molecular weight than expected, but slightly lower than that of an unbounded aggregate. The elution volume of the second minor peak (˜12 mL) corresponded to a protein complex (03) of the expected molecular weight. The elution volume of the major peak (˜14 mL) corresponded to a protein complex larger than that expected for a trimer. DLS of SEC fractions from each peak indicated the formation of aggregates in the first minor peak, monodisperse assemblies with expected hydrodynamic diameters (˜ 35 nm) in the second minor peak, and unassembled trimers in the major peak. Further, nsEM of combined second minor peak fractions confirmed the assembly of homogenous antigen-bearing nanoparticles (FIG. 5E). Additionally, to confirm that large scale expression and purification had not negatively impacted antigenicity, we used BLI to analyze binding of the final purified antigen-bearing nanoparticles. Rpk9_RBD_SARS-COV-2 KWOCA-4 bound CR3022 more tightly than purified monomeric RBD, indicating that the antigen-bearing nanoparticles remained antigenically intact.

Thus, we have provided genetically deliverable nanoparticle vaccines by thoroughly characterizing the biochemical, biophysical, and antigenic properties of mammalian expressed, antigen-bearing secretion-optimized protein nanoparticles.

### Methods

### Plasmid Construction

Wild-type and Rpk9 RBDs were genetically fused to nanoparticles using linkers of 16 glycine and serine residues. All sequences were cloned into pCMV/R using the Xbal and AvrlI restriction sites and Gibson assembly. All antigen-bearing nanoparticles contained an N-terminal mu-phosphatase signal peptide.

### Protein Production (Small Scale)

For small scale mammalian expression and purification of antigen-bearing nanoparticles, Expi293F cells were passaged according to manufacturer protocols (ThermoFisher Scientific). Cells at 3.0×10⁶ cells/mL were transfected with 1 μg/mL cell culture of plasmid DNA with 3 μg/μg PEI-MAX in 70 μL/mL of culture. Cells were harvested at 72 h post-transfection by centrifugation for 5 minutes at 4,100 g, addition of PDADMAC solution to a final concentration of 0.0375% (Sigma Aldrich), a second centrifugation at 5 minutes at 4,100 g, then sterile filtration of supernatant (0.22 μm, Millipore Sigma).

### Biolayer Interferometry (Small Scale)

Binding of CV30 IgG to antigen-bearing nanoparticles was analyzed for antigenicity using an Octet Red™ 96 System (Pall FortéBio/Sartorius) at ambient temperature with shaking at 1000 rpm. Monomeric RBD positive control samples were diluted to 100 nM in Kinetics buffer (Pall FortéBio/Sartorius). Buffer, antibody, receptor, positive control, and cell supernatants were applied to a black 96-well Greiner Bio-one microplate at 200 AL per well. Protein A biosensors were first hydrated for 10 min in Kinetics buffer, then dipped into CV30 diluted to 10 μg/mL in Kinetics buffer in the immobilization step. After 500 s, the tips were transferred to Kinetics buffer for 90 s to reach a baseline. The association step was performed by dipping the loaded biosensors into the immunogens for 300 s, and the subsequent dissociation steps was performed by dipping the biosensors back into Kinetics buffer for an additional 300 s.

### Protein Production and Purification (Large Scale)

For purification of plasmid DNA for large-scale transfection, bacteria were cultured and plasmids were harvested according to the QIAGEN Plasmid Plus™ Maxi Kit™ protocol (QIAGEN). For large scale mammalian expression and purification of antigen-bearing nanoparticles, Expi293F cells were passaged according to manufacturer protocols (ThermoFisher Scientific). Cells at 3.0×10⁶ cells/mL were transfected with 1 μg/mL cell culture of purified plasmid DNA with 3 μg/μg PEI-MAX in 70 μL/mL of culture. Cells were harvested at 72 h post-transfection by centrifugation for 5 minutes at 4,100 g, addition of PDADMAC solution to a final concentration of 0.0375% (Sigma Aldrich), a second centrifugation at 5 minutes at 4,100 g, then sterile filtration of supernatant (0.22 μm, Millipore Sigma). Before lectin affinity chromatography, the filtered supernatant was adjusted to 50 mM Tris (pH 8.0). For each litre of supernatant, 2 ml of Galanthus Nivalis Gel (GNA) immobilized lectin conjugated resin (EY Laboratories) was rinsed into PBS using a gravity column and then added to the supernatant, followed by overnight shaking at 4° C. The resin was collected 16-24 h later using a gravity column, then washed twice with 50 mM Tris (pH 8.0) 150 mM NaCl, 100 mM Arginine (pH 8.0), 5% v/v Glycerol, and 0.02% w/v Sodium azide before elution of antigen-bearing nanoparticles using 50 mM Tris (pH 8.0) 150 mM NaCl, 100 mM Arginine (pH 8.0), 5% v/v Glycerol, 0.02% w/v Sodium azide, and IM Methyl-α-D-mannopyranoside. Eluates were concentrated and applied to a Superose™ 6 Increase 10/300 GL column pre-equilibrated with 50 mM Tris (pH 8.0) 150 mM NaCl, 100 mM Arginine (pH 8.0), 5% v/v Glycerol for preparative size exclusion chromatography (SEC). Peaks corresponding to antigen-bearing nanoparticles were identified based on elution volume. Fractions containing pure antigen-bearing nanoparticles were pooled and quantified using a NanoDrop 8000 Spectrophotometer (ThermoFisher Scientific), then stored at 4° C. until use or flash-frozen in liquid nitrogen and stored at −80° C. Protein content and purity at each step of expression and purification were analyzed by SDS-PAGE using Criterion precast gels and electrophoresis systems (BIO-RAD).

### Dynamic Light Scattering

Dynamic Light Scattering (DLS) was used to measure hydrodynamic diameter (Dh) and % Polydispersity (% Pd) of antigen-bearing nanoparticles on an UNcle™ Nano-DSF (UNchained Laboratories). Sample was applied to a 8.8 μL quartz capillary cassette (UNi, UNchained Laboratories) and measured with 10 acquisitions of 5 s each, using auto-attenuation of the laser. Increased viscosity due to the inclusion of 5% v/v Glycerol in buffer was accounted for by the UNcle™ Client software.

### Negative Stain Electron Microscopy

To image antigen-bearing nanoparticles, protein samples were diluted to 0.050-0.100 mg/ml in 50 mM Tris (pH 8.0), with 150 mM NaCl, 100 mM Arginine (pH 8.0), 5% v/v Glycerol. 400 mesh copper grids (Ted Pella) were glow discharged immediately before use. 3-6 μl of sample was applied to the grid for 1 min, then briefly dipped in a droplet of water before blotting away excess liquid with Whatman no. I filter paper. Grids were stained with 3-6 μl of 0.75-2% w/v uranyl formate stain, immediately blotting away excess, then stained again with another 6 μl for 30 s. Grids were imaged on a Talos L120C transmission electron microscope with a Ceta 4K CCD camera.

### Biolayer Interferometry (Large Scale)

Binding of CR3022 IgG to antigen-bearing nanoparticles was analyzed for antigenicity using an Octet Red™ 96 System (Pall FortéBio/Sartorius) at ambient temperature with sbaking at 1000 rpm. Protein samples were diluted to 100 nM in Kinetics buffer (Pall FortéBio/Sartorius). Buffer, antibody, receptor, and immunogen were then applied to a black 96-well Greiner Bio-one microplate at 200 μL per well. Protein A biosensors were first hydrated for 10 min in Kinetics buffer, then dipped into CR3022 diluted to 10 μg/mL in Kinetics buffer in the immobilization step. After 500 s, the tips were transferred to Kinetics buffer for 90 s to reach a baseline. The association step was performed by dipping the loaded biosensors into the immunogens for 300 s, and the subsequent dissociation steps was performed by dipping the biosensors back into Kinetics buffer for an additional 300 s.

### REFERENCES

Bachmann M F, Jennings G T. Vaccine delivery: a matter of size, geometry, kinetics and molecular patterns. Nat Rev Immunol. 2010; 10:787-796.
Baek M, DiMaio F, Anishchenko I, Dauparas J, Ovchinnikov S, Lee G R, et al. Accurate prediction of protein structures and interactions using a three-track neural network. Science. 2021; 373:871-876.
Bale J B, Gonen S, Liu Y, Sheffler W, Ellis D, et al. Accurate design of megadalton-scale two-component icosahedral protein complexes. Science. 2016. 353, 389-394.
Boyoglu-Barnum S, Ellis D, Gillespie R A, Hutchinson G B, Park Y J, Moin S M, et al. Quadrivalent influenza nanoparticle vaccines induce broad protection. Nature. 2021; 592: 623-628.
Corbett K S, Edwards D K, Leist S R, Abiona O M, Boyoglu-Barnum S, Gillespie R A, et al. SARS-COV-2 mRNA vaccine design enabled by prototype pathogen preparedness. Nature. 2020; 586.
Dauparas J, Anishchenko I, Bennett N, Bai H, Ragotte R J, Milles L F, et al. Robust deep learning-based protein sequence design using ProteinMPNN. Science. 2022; 378:49-56.
Ellis D, Brunette N, Crawford K H D, Walls A C, Pham M N, Chen C, et al. Stabilization of the SARS-COV-2 Spike Receptor-Binding Domain Using Deep Mutational Scanning and Structure-Based Design. Front Immunol. 2021; 12:710263.
Gause K T, Wheatley A K, Cui J, Yan Y, Kent S J, Caruso F. Immunological Principles Guiding the Rational Design of Particles for Vaccine Delivery. ACS Nano. 2017; 11:54-68.
Hsia Y, Bale J B, Gonen S, Shi D, Sheffler W, Fong K K, et al. Design of a hyperstable 60-subunit protein dodecahedron. Nature. 2016; 535:136-139.
Irvine D J, Read B J. Shaping humoral immunity to vaccines through antigen-displaying nanoparticles. Curr Opin Immunol. 2020; 65:1-6.
Jumper J, Evans R, Pritzel A, Green T, Figurnov M, Ronneberger O, et al. Highly accurate protein structure prediction with AlphaFold. Nature. 2021; 596:583-589.
Kanekiyo M, Ellis D, King N P. New Vaccine Design and Delivery Technologies. J Infect Dis. 2019; 219: S88-S96.
King N P, Bale J B, Sheffler W, Mcnamara D E, Gonen S, Gonen T, et al. Accurate design of co-assembling multi-component protein nanomaterials. Nature. 2014. 510 (7503): 103-8
King N P, Sheffler W, Sawaya M R, Vollmar B S, Sumida J P, André I, et al. Computational design of self-assembling protein nanomaterials with atomic level accuracy. Science. 2012; 336:1171-1174.
López-Sagaseta J, Malito E, Rappuoli R, Bottomley M J. Self-assembling protein nanoparticles in the design of vaccines. Comput Struct Biotechnol J. 2016; 14:58-68.
Marcandalli J, Fiala B, Ols S, Perotti M, de van der Schueren W, Snijder J, et al. Induction of Potent Neutralizing Antibody Responses by a Designed Protein Nanoparticle Vaccine for Respiratory Syncytial Virus. Cell. 2019; 176:1420-1431.e17.
Vogel A B, Kanevsky I, Che Y, Swanson K A, Maik A, Vormehr M, et al. BNT162b vaccines protect rhesus macaques from SARS-COV-2. Nature. 2021; 592:283-289.
Walls A C, Fiala B, Schäfer A, Wrenn S, Pham M N, Murphy M, et al. Elicitation of Potent Neutralizing Antibody Responses by Designed Protein Nanoparticle Vaccines for SARS-COV-2. Cell. 2020; 183:1367-1382.e17.
Wang J Y, Khmelinskaia A, Sheffler W, Miranda M C, Antanasijevic A, Borst A J, et al. Improving the secretion of designed protein assemblies through negative design of cryptic transmembrane domains. PNAS. 2023;120 (11): e2214556120.

1. A polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to, and identical at least at one identified interface position, to the amino acid sequence selected from the group consisting of SEQ ID NO:1-44, wherein residues in parentheses are optional, and may be present or absent; wherein any N-terminal methionine residues are optional and may be present or absent; and wherein some or all of the optional residues may be absent and not included for determining percent identity.

2. The polypeptide of claim 1, wherein the polypeptide is identical at least at two identified interface positions relative to the reference amino acid sequence.

3-5. (canceled)

6. A polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:45-58, wherein residues in parentheses are optional, and may be present or absent; wherein any N-terminal methionine residues are optional and may be present or absent; wherein some or all of the optional residues may be absent and not included for determining percent identity.

7-11. (canceled)

12. A fusion protein, comprising:
(a) the polypeptide of claim 1;
(b) one or more additional polypeptides; and
(c) optional amino acid linkers between the polypeptide and the one or more additional polypeptides.

13. The fusion protein of claim 12, wherein the one or more additional polypeptides comprise an antigen.

14. The fusion protein of claim 13, wherein the antigen comprises a bacterial or viral antigen.

15. The fusion protein of claim 14, wherein the bacterial or viral antigen comprises a coronavirus antigen, including but not limited to a SARS COV-2 antigen.

16. The fusion protein of claim 15, wherein the coronavirus antigen comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 59-70.

17. (canceled)

18. A nucleic acid encoding the polypeptide of claim 1.

19-26. (canceled)

27. An expression vector, comprising the nucleic acid of claim 18 operatively linked to a suitable control sequence.

28. A host cell comprising the expression vector of claim 27.

29. A nanoparticle comprising a plurality of the polypeptides of claim 1.

30. The nanoparticle of claim 29, wherein all of the polypeptides are fused to a polypeptide antigen, wherein the polypeptide antigen may be identical in all of the polypeptides or fusion proteins, or wherein the nanoparticle may present more than one polypeptide antigen.

31. The nanoparticle of claim 29, wherein only a portion of the polypeptides are fused to a polypeptide antigen, wherein the polypeptide antigen present may be identical in all cases, or wherein the nanoparticle may present more than one polypeptide antigen.

32. A pharmaceutical composition or vaccine comprising
(a) the fusion protein of claim 13; and
(b) a pharmaceutically acceptable carrier.

33-35. (canceled)

36. A method for treating or limiting development of an infection, comprising administering to a subject an amount effective to treat or limiting development of the infection of the fusion protein of claim 13.

37-39. (canceled)


SEQUENCE LISTING (91 sequences)

SEQ ID NO: 1 - protein, 255 aa, synthetic construct
  REGION 1..29: note = optionally absent
MHHHHHHGGSEQKLISEEDLSGGGSWSGSTEVEKKAREVAKEAVELASLLRSETAIRVAQAILEAAEAAKRAAEQGKTEVAKLALKVLEEAIELAKEKRSEEALKVVLEIARAALAAAQAAEEGFTDVAKMALEVLERAIELAKDDRSEEALKEVLEIARAALAAAQLAKKGRDDEARKILMKLRIRITLRKLEESLRELRRILEELKEMLERLEKNPDKDVIVKVLKVIVKAIEASVENQRISAENQKALAELA

SEQ ID NO: 2 - protein, 277 aa, synthetic construct
  REGION 1..51: note = optionally absent
METDTLLLWVLLLWVPGSTGDGSHHHHHHGGSEQKLISEEDLSGGGSWSGSTEVEKKAREVAKEAVELASLLRSETAIRVAQAILEAAEAAKRAAEQGKTEVAKLALKVLEEAIELAKEKRSEEALKVVLEIARAALAAAQAAEEGFTDVAKMALEVLERAIELAKDDRSEEALKEVLEIARAALAAAQLAKKGRDDEARKILMKLRIRITLRKLEESLRELRRILEELKEMLERLEKNPDKDVIVKVLKVIVKAIEASVENQRISAENQKALAELA

SEQ ID NO: 3 - protein, 264 aa, synthetic construct
  UNSURE 1..29: note = optionally absent
MHHHHHHGGSEQKLISEEDLSGGGSWSGSSDEEEAREWAERALKAALEAAEQALREGDEDAFKCAVELLEQALEARKKKDSEEAEAVYWAARAVLAALEALEQAKREGDEDARRCAEELLRLACEAARKKNSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 4 - protein, 286 aa, synthetic construct
  UNSURE 1..51: note = optionally absent
METDTLLLWVLLLWVPGSTGDGSHHHHHHGGSEQKLISEEDLSGGGSWSGSSDEEEAREWAERALKAALEAAEQALREGDEDAFKCAVELLEQALEARKKKDSEEAEAVYWAARAVLAALEALEQAKREGDEDARRCAEELLRLACEAARKKNSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 5 - protein, 284 aa, synthetic construct
  UNSURE 1..29: note = optionally absent
MHHHHHHGGSEQKLISEEDLSGGGSWSGSTEEKIEEARQAIKEAERALREGDPRFAEMAVRIALALVRMLERLARKTGSTEVLIEAARLAIEVARVALKVGSPETAREAVRTALELVQELERQARKTGSTEVLIEAARLAIEVARVAFKVGSPETAKEAVRTALELVKELIQQALKTGSDEVLERAAELAKEVARVAKEVGDPRAARKADMVAKIADTLRELMESLRELRRILEELKEMLERLEKNPDKDVIVKVLKVIVKAIEASVENQRISAENQAALASLA

SEQ ID NO: 6 - protein, 306 aa, synthetic construct
  UNSURE 1..51: note = optionally absent
METDTLLLWVLLLWVPGSTGDGSHHHHHHGGSEQKLISEEDLSGGGSWSGSTEEKIEEARQAIKEAERALREGDPRFAEMAVRIALALVRMLERLARKTGSTEVLIEAARLAIEVARVALKVGSPETAREAVRTALELVQELERQARKTGSTEVLIEAARLAIEVARVAFKVGSPETAKEAVRTALELVKELIQQALKTGSDEVLERAAELAKEVARVAKEVGDPRAARKADMVAKIADTLRELMESLRELRRILEELKEMLERLEKNPDKDVIVKVLKVIVKAIEASVENQRISAENQAALASLA

SEQ ID NO: 7 - protein, 309 aa, synthetic construct
  UNSURE 1: note = optionally absent
  UNSURE 289..309: note = optionally absent
MADELRAVAELQRLNIELARKLLEAVARLQELNIDLVRKTSELTDEKTIREEIRKVKEESKRIVEEAEEEIRRAKEDSKRIVTEALRRAREQIREKWEELEERAKRAETPEEALRAAEEMVKLIEELIRIAEMLQRAGLKEEAEDVLREATELIKRATELLEKIAKNSDTPELALRAAELLVRLIKLLIEIAKLLQEQGNKEEAEKVLREATELIKRVARLLLAIALLADTPELAKRAAELLKRLIELLKEIAKLLEEEGNEDEAEKVKEEAKELEELVRWLEEQIRGGSWSGGGSEQKLISEEDLGGS

SEQ ID NO: 8 - protein, 331 aa, synthetic construct
  UNSURE 1..23: note = optionally absent
  UNSURE 311..331: note = optionally absent
METDTLLLWVLLLWVPGSTGDGSADELRAVAELQRLNIELARKLLEAVARLQELNIDLVRKTSELTDEKTIREEIRKVKEESKRIVEEAEEEIRRAKEDSKRIVTEALRRAREQIREKWEELEERAKRAETPEEALRAAEEMVKLIEELIRIAEMLQRAGLKEEAEDVLREATELIKRATELLEKIAKNSDTPELALRAAELLVRLIKLLIEIAKLLQEQGNKEEAEKVLREATELIKRVARLLLAIALLADTPELAKRAAELLKRLIELLKEIAKLLEEEGNEDEAEKVKEEAKELEELVRWLEEQIRGGSWSGGGSEQKLISEEDLGGS

SEQ ID NO: 9 - protein, 264 aa, synthetic construct
  UNSURE 1..29: note = optionally absent
MHHHHHHGGSEQKLISEEDLSGGGSWSGSSDEEEAREWAERAEEAAKEALEQAKREGDEIARLCAKMLEILAEEARRKKDSEEAEAVYWAARAVLAALEALEQAKREGDEDARRCAEELLRLACSAAARQDSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 10 - protein, 286 aa, synthetic construct
  UNSURE 1..51: note = optionally absent
METDTLLLWVLLLWVPGSTGDGSHHHHHHGGSEQKLISEEDLSGGGSWSGSSDEEEAREWAERAEEAAKEALEQAKREGDEIARLCAKMLEILAEEARRKKDSEEAEAVYWAARAVLAALEALEQAKREGDEDARRCAEELLRLACSAAARQDSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 11 - protein, 286 aa, synthetic construct
  UNSURE 1: note = optionally absent
  UNSURE 266..286: note = optionally absent
MDECERLETEVMKAAKELMKLATQSTDKEVRKIAWEVADQLLRLAEEACRSNSDECLRLASEVVKAVQELVKLAEQATDEEVIRVALEVARELIRLAQEACRSNDDECLRLASEVVKAVQEAVKLAEQAKDERVIEVALEMARLLIELAQEACRRNDEEALRRASEIVKRVQELIKEAEKATDEEEIERLLRKAAIDITLAQLEISLKELRRILEELKEMLERLEKNPDKDVIVKVLKVIVKAIEASVENQRISAENQKMLAELAGSWSGGGSEQKLISEEDLGGS

SEQ ID NO: 12 - protein, 308 aa, synthetic construct
  UNSURE 1..23: note = optionally absent
  UNSURE 288..308: note = optionally absent
METDTLLLWVLLLWVPGSTGDGSDECERLETEVMKAAKELMKLATQSTDKEVRKIAWEVADQLLRLAEEACRSNSDECLRLASEVVKAVQELVKLAEQATDEEVIRVALEVARELIRLAQEACRSNDDECLRLASEVVKAVQEAVKLAEQAKDERVIEVALEMARLLIELAQEACRRNDEEALRRASEIVKRVQELIKEAEKATDEEEIERLLRKAAIDITLAQLEISLKELRRILEELKEMLERLEKNPDKDVIVKVLKVIVKAIEASVENQRISAENQKMLAELAGSWSGGGSEQKLISEEDLGGS

SEQ ID NO: 13 - protein, 332 aa, synthetic construct
  UNSURE 1..29: note = optionally absent
MHHHHHHGGSEQKLISEEDLSGGGSWSGSTEEKIAKEISRIAEESKKAIETLARLADKMTDENQVDTAIELIAKIAREAIKRIEDLAKNLASEEFMARAISAIAELAKKAIEAIYRLAELHRTDTFMAKAIEAIAELAKEAIKAIADLAKKHTTEEFMARAISAIAELAKKAIEAIWRLASLHKTDEFMDKAAEAIAELAEEAIRAIRELAKKHTTEEFVRKAESAVREISKKAKDAIRKLADAMRDPTAREKAKKLEIKVELAEALAELAVALLKLKLSLDELERSLEELEKNPSEDALVENNRLNVENNKIIVEVLRIIAEVLDINAQLV

SEQ ID NO: 14 - protein, 354 aa, synthetic construct
  UNSURE 1..51: note = optionally absent
METDTLLLWVLLLWVPGSTGDGSHHHHHHGGSEQKLISEEDLSGGGSWSGSTEEKIAKEISRIAEESKKAIETLARLADKMTDENQVDTAIELIAKIAREAIKRIEDLAKNLASEEFMARAISAIAELAKKAIEAIYRLAELHRTDTFMAKAIEAIAELAKEAIKAIADLAKKHTTEEFMARAISAIAELAKKAIEAIWRLASLHKTDEFMDKAAEAIAELAEEAIRAIRELAKKHTTEEFVRKAESAVREISKKAKDAIRKLADAMRDPTAREKAKKLEIKVELAEALAELAVALLKLKLSLDELERSLEELEKNPSEDALVENNRLNVENNKIIVEVLRIIAEVLDINAQLV

SEQ ID NO: 15 - protein, 273 aa, synthetic construct
  UNSURE 1: note = optionally absent
  UNSURE 253..273: note = optionally absent
MDESVDLAVKLAEALRKEAEELIKKARKTGDPELLRKALEALEKAVKLVEDAIKRNPDNDEAVETAVRLARELKKVAEELQERAKKTGDPELLKLALRALEVAVRAVELAIKSNPDNDEAVKTAVELAKELEKVARELLERARKTGDDELLKLAKRALEVARRAVELALKSRPDAEEARRVYIRLTEMELEISLTELRKILEELKEMLERLEKNPDKDVIVKVLKVIVKAIEASVENQRISAENQKALAELAGSWSGGGSEQKLISEEDLGGS

SEQ ID NO: 16 - protein, 295 aa, synthetic construct
  UNSURE 1..23: note = optionally absent
  UNSURE 275..295: note = optionally absent
METDTLLLWVLLLWVPGSTGDGSDESVDLAVKLAEALRKEAEELIKKARKTGDPELLRKALEALEKAVKLVEDAIKRNPDNDEAVETAVRLARELKKVAEELQERAKKTGDPELLKLALRALEVAVRAVELAIKSNPDNDEAVKTAVELAKELEKVARELLERARKTGDDELLKLAKRALEVARRAVELALKSRPDAEEARRVYIRLTEMELEISLTELRKILEELKEMLERLEKNPDKDVIVKVLKVIVKAIEASVENQRISAENQKALAELAGSWSGGGSEQKLISEEDLGGS

SEQ ID NO: 17 - protein, 297 aa, synthetic construct
  UNSURE 1..29: note = optionally absent
MHHHHHHGGSEQKLISEEDLSGGGSWSGSDEEVWKAVIDAIELMKEARELIKKARKTGDPELLRKALEALEEAVRAVEEAIKRNPDNKIAVIVAVLLARELKKVAEELQERAKKTGDPELLKLALRALEVAVRAVELAIKSNPDNDEAVETAVRLAEELAKVAKELIERAKKTGDADLLRLAKRAIEVARRAVELAKKSRPDAERADEAYKRLKELEREIRELLRKMLTEALRKLEKALQELREMLRKLKESLEELKKNPSEDALVRNNELIVEVLRVIVEVLSIIAEVLKINAALV

SEQ ID NO: 18 - protein, 319 aa, synthetic construct
  UNSURE 1..51: note = optionally absent
METDTLLLWVLLLWVPGSTGDGSHHHHHHGGSEQKLISEEDLSGGGSWSGSDEEVWKAVIDAIELMKEARELIKKARKTGDPELLRKALEALEEAVRAVEEAIKRNPDNKIAVIVAVLLARELKKVAEELQERAKKTGDPELLKLALRALEVAVRAVELAIKSNPDNDEAVETAVRLAEELAKVAKELIERAKKTGDADLLRLAKRAIEVARRAVELAKKSRPDAERADEAYKRLKELEREIRELLRKMLTEALRKLEKALQELREMLRKLKESLEELKKNPSEDALVRNNELIVEVLRVIVEVLSIIAEVLKINAALV

SEQ ID NO: 19 - protein, 284 aa, synthetic construct
  UNSURE 1..29: note = optionally absent
MHHHHHHGGSEQKLISEEDLSGGGSWSGSTEETIEMARQLIKEAERALREGDPEEARMAVEMALAAVRILERQARKTGSTEVLIEAARLAIEVARVALKVGSPETAREAVRTALELVQELERQARKTGSTEVLIEAARLAIEVARVAFKVGSPETAKEAVRTALELVKELIQQALKTGSDEVLERAAELAKEVARVAKEVGDPRAARKADMVAKIADTLRELMESLRELRRILEELKEMLERLEKNPDKDVIVKVLKVIVKAIEASVENQRISAENQAALASLA

SEQ ID NO: 20 - protein, 306 aa, synthetic construct
  UNSURE 1..51: note = optionally absent
METDTLLLWVLLLWVPGSTGDGSHHHHHHGGSEQKLISEEDLSGGGSWSGSTEETIEMARQLIKEAERALREGDPEEARMAVEMALAAVRILERQARKTGSTEVLIEAARLAIEVARVALKVGSPETAREAVRTALELVQELERQARKTGSTEVLIEAARLAIEVARVAFKVGSPETAKEAVRTALELVKELIQQALKTGSDEVLERAAELAKEVARVAKEVGDPRAARKADMVAKIADTLRELMESLRELRRILEELKEMLERLEKNPDKDVIVKVLKVIVKAIEASVENQRISAENQAALASLA

SEQ ID NO: 21 - protein, 264 aa, synthetic construct
  UNSURE 1..29: note = optionally absent
MHHHHHHGGSEQKLISEEDLSGGGSWSGSSDEEEAREWAERAEEAAKEALEQAKREGDEIARLCAEMLEILAEEARRKKDSEEAEAVYWAARATLAALEALEQAKREGDEDARRCAEELLRLACSAAARQDSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 22 - protein, 286 aa, synthetic construct
  UNSURE 1..51: note = optionally absent
METDTLLLWVLLLWVPGSTGDGSHHHHHHGGSEQKLISEEDLSGGGSWSGSSDEEEAREWAERAEEAAKEALEQAKREGDEIARLCAEMLEILAEEARRKKDSEEAEAVYWAARATLAALEALEQAKREGDEDARRCAEELLRLACSAAARQDSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 23 - protein, 277 aa, synthetic construct
  UNSURE 1: note = optionally absent
  UNSURE 257..277: note = optionally absent
MMTEEKIEEARQSIKEAERSLREGNPEKALDAVARALSLVNELERLARKTGSTEVLIEAARLAIEVARVALKVGSPEMAQLAVELALRLVQELERQARKTGSTEVLIEAARLAIEVARVAFKVGSPETAREAARTALELVEELERQARKTGSEEVLERAARLAEEVARVAEEIGDPELARKAMKVAIRLTEELLKKSLRELRRILEELKEMLERLEKNPDKDVIVKVLKVIVKAIEASVENQRISADNQRALARLAGSWSGGGSEQKLISEEDLGGS

SEQ ID NO: 24 - protein, 298 aa, synthetic construct
  UNSURE 1..23: note = optionally absent
  UNSURE 278..298: note = optionally absent
METDTLLLWVLLLWVPGSTGDGSTEEKIEEARQSIKEAERSLREGNPEKALDAVARALSLVNELERLARKTGSTEVLIEAARLAIEVARVALKVGSPEMAQLAVELALRLVQELERQARKTGSTEVLIEAARLAIEVARVAFKVGSPETAREAARTALELVEELERQARKTGSEEVLERAARLAEEVARVAEEIGDPELARKAMKVAIRLTEELLKKSLRELRRILEELKEMLERLEKNPDKDVIVKVLKVIVKAIEASVENQRISADNQRALARLAGSWSGGGSEQKLISEEDLGGS

SEQ ID NO: 25 - protein, 233 aa, synthetic construct
  UNSURE 1: note = optionally absent
  UNSURE 226..233: note = optionally absent
MSDEVDRRVLELAIKASRATDKEEVIEIVKELAELAKQSTDSRLVERIVTLLALVAIDATDKELVIYIVKILAELAKQSTDSELVKKIVEMLAQVARFATDKELVEYIARILLELAKQADDATLVAFIAEMLAEVRKEAKDKELKEKIDEILKELAKITLKALEDSLRELRRILEELKEMLERLEKNPDKDVIVKVLKVIVKAIEASVKNQEISAANQKALALLGLEHHHHHH

SEQ ID NO: 26 - protein, 259 aa, synthetic construct
  UNSURE 1..23: note = optionally absent
  UNSURE 248..259: note = optionally absent
METDTLLLWVLLLWVPGSTGDGSSDEVDRRVLELAIKASRATDKEEVIEIVKELAELAKQSTDSRLVERIVTLLALVAIDATDKELVIYIVKILAELAKQSTDSELVKKIVEMLAQVARFATDKELVEYIARILLELAKQADDATLVAFIAEMLAEVRKEAKDKELKEKIDEILKELAKITLKALEDSLRELRRILEELKEMLERLEKNPDKDVIVKVLKVIVKAIEASVKNQEISAANQKALALLGGSEQKLISEEDL

SEQ ID NO: 27 - protein, 329 aa, synthetic construct
  UNSURE 1: note = optionally absent
  UNSURE 309..329: note = optionally absent
MIMSKIAETAKRLADSLRELRRILEELKEMLERLEKRPDKKVIVDVLKVIVKAIEASVENQRISASNQAALALAIAAEAVKEIEEDIDRARKLKDEGNKEEAEKVLRKAREKIREVRDALDAIAKGAGTPDIALKAAELLVRLIKLLIEIAKLLQDAGNKEEAEKVLREATELIKRVTELLEKIAKNSDTPELALRAAELLVRLIKLLIEIAKLLQEQGNKEEAEKVLREATKMIIRVAQLLVKIAKNSDEPELAKRAAELLKRLIELLKEIAKLLEEEGNEDEAEKVKEIAKILEEAVRELEERIIGGSWSGGGSEQKLISEEDLGGS

SEQ ID NO: 28 - protein, 351 aa, synthetic construct
  UNSURE 1..23: note = optionally absent
  UNSURE 331..351: note = optionally absent
METDTLLLWVLLLWVPGSTGDGSIMSKIAETAKRLADSLRELRRILEELKEMLERLEKRPDKKVIVDVLKVIVKAIEASVENQRISASNQAALALAIAAEAVKEIEEDIDRARKLKDEGNKEEAEKVLRKAREKIREVRDALDAIAKGAGTPDIALKAAELLVRLIKLLIEIAKLLQDAGNKEEAEKVLREATELIKRVTELLEKIAKNSDTPELALRAAELLVRLIKLLIEIAKLLQEQGNKEEAEKVLREATKMIIRVAQLLVKIAKNSDEPELAKRAAELLKRLIELLKEIAKLLEEEGNEDEAEKVKEIAKILEEAVRELEERIIGGSWSGGGSEQKLISEEDLGGS

SEQ ID NO: 29 - protein, 280 aa, synthetic construct
  UNSURE 1: note = optionally absent
  UNSURE 260..280: note = optionally absent
MAELAIEMARQSIREAERSLLEGNPEKAREDVRRALELVRLLEKIARREGSTEVLIEAARLAIEVARVALWVGSPETAREAVRTALELVQELERQARKTGSTEVLIEAARLAIEVARVAFEVGSPETAREAARTALELVEELDRQAEKTGSKEVLERAARLAKEVARVAKEIGDPELARKADEVAERLDIKRTLLDLEDSLRELRRILEELKRALEMLEKLPDKEMIRDVLKVIVKAIEASVENQRISAENQKALARLAGSWSGGGSEQKLISEEDLGGS

SEQ ID NO: 30 - protein, 302 aa, synthetic construct
  UNSURE 1..23: note = optionally absent
  UNSURE 282..302: note = optionally absent
METDTLLLWVLLLWVPGSTGDGSAELAIEMARQSIREAERSLLEGNPEKAREDVRRALELVRLLEKIARREGSTEVLIEAARLAIEVARVALWVGSPETAREAVRTALELVQELERQARKTGSTEVLIEAARLAIEVARVAFEVGSPETAREAARTALELVEELDRQAEKTGSKEVLERAARLAKEVARVAKEIGDPELARKADEVAERLDIKRTLLDLEDSLRELRRILEELKRALEMLEKLPDKEMIRDVLKVIVKAIEASVENQRISAENQKALARLAGSWSGGGSEQKLISEEDLGGS

SEQ ID NO: 31 - protein, 253 aa, synthetic construct
  UNSURE 1..29: note = optionally absent
MHHHHHHGGSEQKLISEEDLSGGGSWSGSSKEVTERVAELAAEAVRATDKEEVIEIVKELAELAKQSTDSELVNFIVRALAAVAIAAQDKELVIYIVKILAELAKQSTDSELVNEIVKALAEVAKAATDKELVKYIVDILLELAKQADDATLVAKIAEQLAEVREEAKDKELQERIDRVLKKLIEITLRALEESLRELRRILEELKEMLERLEKNPDKDVIVKVLKVIVKAIEASVRNQQISAANQKALALLA

SEQ ID NO: 32 - protein, 275 aa, synthetic construct
  UNSURE 1..51: note = optionally absent
METDTLLLWVLLLWVPGSTGDGSHHHHHHGGSEQKLISEEDLSGGGSWSGSSKEVTERVAELAAEAVRATDKEEVIEIVKELAELAKQSTDSELVNFIVRALAAVAIAAQDKELVIYIVKILAELAKQSTDSELVNEIVKALAEVAKAATDKELVKYIVDILLELAKQADDATLVAKIAEQLAEVREEAKDKELQERIDRVLKKLIEITLRALEESLRELRRILEELKEMLERLEKNPDKDVIVKVLKVIVKAIEASVRNQQISAANQKALALLA

SEQ ID NO: 33 - protein, 235 aa, synthetic construct
  UNSURE 1: note = optionally absent
  UNSURE 215..235: note = optionally absent
MSSEEAERIARILEEVWSPDPENIREAVRKAEELLRENPSRQAEELLREAIEAAVRAPDPEAIREAVRAAEELLRENPSTEAEELLRRAIEAAVRAPDPEAIREAVRAARELFKENPSEEAEELLKRAADSALKAPDPRAIREALEALLELLEAALRRLKKSLDELERSLEELEKNPSEDALVENNRLNVENNKIIVKVLEIIARVLKANARLVGSWSGGGSEQKLISEEDLGGS

SEQ ID NO: 34 - protein, 257 aa, synthetic construct
  UNSURE 1..23: note = optionally absent
  UNSURE 237..257: note = optionally absent
METDTLLLWVLLLWVPGSTGDGSSSEEAERIARILEEVWSPDPENIREAVRKAEELLRENPSRQAEELLREAIEAAVRAPDPEAIREAVRAAEELLRENPSTEAEELLRRAIEAAVRAPDPEAIREAVRAARELFKENPSEEAEELLKRAADSALKAPDPRAIREALEALLELLEAALRRLKKSLDELERSLEELEKNPSEDALVENNRLNVENNKIIVKVLEIIARVLKANARLVGSWSGGGSEQKLISEEDLGGS

SEQ ID NO: 35 - protein, 248 aa, synthetic construct
  UNSURE 1: note = optionally absent
  UNSURE 228..248: note = optionally absent
MALEKDRRALEALKRAQEAEKKGDVEEAVRAAQEAVRAAKESGASWILRLVAEQALRIAKEAEKQGNVEVAVKAARVAVEAAKQAGDNDVLRKVAEQALRIAKEAEKQGNVDVAAKAAQVAAEAAKQAGDKDMLEKVAKVAEQIAKAAEKEGDKKVSIDATRIALEASLAALEIILEELKEMLERLEKNPDKDVIVKVLKVIVKAIEASVKNQKISAKNQKALAELAGSWSGGGSEQKLISEEDLGGS

SEQ ID NO: 36 - protein, 270 aa, synthetic construct
  UNSURE 1..23: note = optionally absent
  UNSURE 250..270: note = optionally absent
METDTLLLWVLLLWVPGSTGDGSALEKDRRALEALKRAQEAEKKGDVEEAVRAAQEAVRAAKESGASWILRLVAEQALRIAKEAEKQGNVEVAVKAARVAVEAAKQAGDNDVLRKVAEQALRIAKEAEKQGNVDVAAKAAQVAAEAAKQAGDKDMLEKVAKVAEQIAKAAEKEGDKKVSIDATRIALEASLAALEIILEELKEMLERLEKNPDKDVIVKVLKVIVKAIEASVKNQKISAKNQKALAELAGSWSGGGSEQKLISEEDLGGS

SEQ ID NO: 37 - protein, 316 aa, synthetic construct
  UNSURE 1..29: note = optionally absent
MHHHHHHGGSEQKLISEEDLSGGGSWSGSPRERLEEAKERVEEIRELIDKARKLQEQGDRIRATAVLMEARAQIEEVTRELEEIAKNSDTPELALRAAELLVRLIKLLIEIAKLLQEQGQTQSAEDVLRQATELIKRVTELLEKIAKNSDTPELALRAAELLVRLIKLLIEIAKLLQEQGNKEEATKVLREAEELIERVFELLKKIAENSDTPELAKRAEELIERLIELLEEIAKLLEEAGRRKEALRVLLKALELLLRLLKKSLDELERSLEELEKNPSEDALVENNRLNVKNNRIIVKVLEMIAKVLKMNAKAV

SEQ ID NO: 38 - protein, 338 aa, synthetic construct
  UNSURE 1..51: note = optionally absent
METDTLLLWVLLLWVPGSTGDGSHHHHHHGGSEQKLISEEDLSGGGSWSGSPRERLEEAKERVEEIRELIDKARKLQEQGDRIRATAVLMEARAQIEEVTRELEEIAKNSDTPELALRAAELLVRLIKLLIEIAKLLQEQGQTQSAEDVLRQATELIKRVTELLEKIAKNSDTPELALRAAELLVRLIKLLIEIAKLLQEQGNKEEATKVLREAEELIERVFELLKKIAENSDTPELAKRAEELIERLIELLEEIAKLLEEAGRRKEALRVLLKALELLLRLLKKSLDELERSLEELEKNPSEDALVENNRLNVKNNRIIVKVLEMIAKVLKMNAKAV

SEQ ID NO: 39 - protein, 249 aa, synthetic construct
  UNSURE 1: note = optionally absent
  UNSURE 229..249: note = optionally absent
MMALEKDRRALEALRRAQEAEKKGDVEEAVRAAQEAVRAAKESGASWILRLVAEQALRIAKEAEKQGNVEVAVKAARVAVEAAKQAGDNDVLRKVAEQALRIAKEAEKQGNVDVAAKAAQVAAEAAKQAGDKDMLEKVAKVAEQIAKAAEKEGDKKVSIDATRIALEASLAALEIILEELKEMLERLEKNPDKDVIVKVLKVIVKAIEASVKNQKISAKNQKALAELAGSWSGGGSEQKLISEEDLGGS

SEQ ID NO: 40 - protein, 270 aa, synthetic construct
  UNSURE 1..23: note = optionally absent
  UNSURE 250..270: note = optionally absent
METDTLLLWVLLLWVPGSTGDGSALEKDRRALEALRRAQEAEKKGDVEEAVRAAQEAVRAAKESGASWILRLVAEQALRIAKEAEKQGNVEVAVKAARVAVEAAKQAGDNDVLRKVAEQALRIAKEAEKQGNVDVAAKAAQVAAEAAKQAGDKDMLEKVAKVAEQIAKAAEKEGDKKVSIDATRIALEASLAALEIILEELKEMLERLEKNPDKDVIVKVLKVIVKAIEASVKNQKISAKNQKALAELAGSWSGGGSEQKLISEEDLGGS

SEQ ID NO: 41 - protein, 308 aa, synthetic construct
  UNSURE 1..29: note = optionally absent
MHHHHHHGGSEQKLISEEDLSGGGSWSGSGRELKQLAEVLEEIQRLAEEARKLMTDEEEAKKIQEEAERAKRMLASAVWAVTDNEVIEKLLEVVKEIIRLAEEAMKKMTDEEEAAKIAKEALEAIKMLARAVEEVTDNEVIEKLLEVVKEIIRAAEEAMKLMRNEEEAAKIAKKALEAIKALAEAVEAIKNKEEIERLLTLVKELIRKAEEEARRMSDREKAAEIIERALEKIKKLAKLAKALADLEKALRELKKSLDELERSLEELERNPSEAALVENNRLNVENNKIIVEVLRIIAEVLKINAELA

SEQ ID NO: 42 - protein, 330 aa, synthetic construct
  UNSURE 1..51: note = optionally absent
METDTLLLWVLLLWVPGSTGDGSHHHHHHGGSEQKLISEEDLSGGGSWSGSGRELKQLAEVLEEIQRLAEEARKLMTDEEEAKKIQEEAERAKRMLASAVWAVTDNEVIEKLLEVVKEIIRLAEEAMKKMTDEEEAAKIAKEALEAIKMLARAVEEVTDNEVIEKLLEVVKEIIRAAEEAMKLMRNEEEAAKIAKKALEAIKALAEAVEAIKNKEEIERLLTLVKELIRKAEEEARRMSDREKAAEIIERALEKIKKLAKLAKALADLEKALRELKKSLDELERSLEELERNPSEAALVENNRLNVENNKIIVEVLRIIAEVLKINAELA

SEQ ID NO: 43 - protein, 240 aa, synthetic construct
  UNSURE 1..29: note = optionally absent
MHHHHHHGGSEQKLISEEDLSGGGSWSGSTKEDARSTCEKAARKAAESNDEEVAKRAAIECARVAMEAGMPTKEAARSFCEAAARAAAESNDEEVAKIAAKACLIVARAAGMPTEEAARSFCEAAAKAAAEAGDARVAKIAEKACREVARQAGMPEKDADRAFKEAMKQAIEETLKRLEDSLRELRRILEELKEMLERLEKNPDKDVIVKVLKVIVKAIEASVENQRISAKNQAALAALA

SEQ ID NO: 44 - protein, 262 aa, synthetic construct
  UNSURE 1..51: note = optionally absent
METDTLLLWVLLLWVPGSTGDGSHHHHHHGGSEQKLISEEDLSGGGSWSGSTKEDARSTCEKAARKAAESNDEEVAKRAAIECARVAMEAGMPTKEAARSFCEAAARAAAESNDEEVAKIAAKACLIVARAAGMPTEEAARSFCEAAAKAAAEAGDARVAKIAEKACREVARQAGMPEKDADRAFKEAMKQAIEETLKRLEDSLRELRRILEELKEMLERLEKNPDKDVIVKVLKVIVKAIEASVENQRISAKNQAALAALA

SEQ ID NO: 45 - protein, 331 aa, synthetic construct
  UNSURE 1..29: note = optionally absent
MHHHHHHGGSEQKLISEEDLSGGGSWSGSPRLLLEEAKERVETIERLIFTAKALQAKGNKEEAEKVLREAREQIREVTLILELIAKDSDTPELATRAAELLVRLIKLLIEIAKLLQEQGNKEEAEKVLREATELIKRVTELLEKIAKNSDTPELALRAAELLVRLIKLLIEIAKLLQEQGNKEEAEKVLREAEELIKRVLELLEKIAENADTEELARRAAELIKRLIELLKEIAKLLEEAGKKDEAEKVKEKAKEMKERVDILETLIELERSLRELRRILEELKEMLERLERNPDKDVIVKVLKVIVKAIEASVENQRISAENQKALARLA

SEQ ID NO: 46 - protein, 353 aa, synthetic construct
  UNSURE 1..51: note = optionally absent
METDTLLLWVLLLWVPGSTGDGSHHHHHHGGSEQKLISEEDLSGGGSWSGSPRLLLEEAKERVETIERLIFTAKALQAKGNKEEAEKVLREAREQIREVTLILELIAKDSDTPELATRAAELLVRLIKLLIEIAKLLQEQGNKEEAEKVLREATELIKRVTELLEKIAKNSDTPELALRAAELLVRLIKLLIEIAKLLQEQGNKEEAEKVLREAEELIKRVLELLEKIAENADTEELARRAAELIKRLIELLKEIAKLLEEAGKKDEAEKVKEKAKEMKERVDILETLIELERSLRELRRILEELKEMLERLERNPDKDVIVKVLKVIVKAIEASVENQRISAENQKALARLA

SEQ ID NO: 47 - protein, 248 aa, synthetic construct
  UNSURE 228..248: note = optionally absent
MSYDERARKAVKRYVKEEGGSEEEAEREAEKVREEIRKKASDKYLIQAAAAVVAYVIELGGSPDEAVKLAEAVVRAIKAAADDSYLEQAAAAVVAFVIRKGGSPMEAVIKAKEVVDRIKEAADSREATRKAARMVATVIQAGGSPEEAVKDAKKLVDLLRALRELEKALRELKKSLDELERSLEELEKNPSEDALVENNRLNVENNKIIVEVLRIIAEVLKINAELVGSWSGGGSEQKLISEEDLGGS

SEQ ID NO: 48 - protein, 270 aa, synthetic construct
  UNSURE 1..23: note = optionally absent
  UNSURE 250..270: note = optionally absent
METDTLLLWVLLLWVPGSTGDGSSYDERARKAVKRYVKEEGGSEEEAEREAEKVREEIRKKASDKYLIQAAAAVVAYVIELGGSPDEAVKLAEAVVRAIKAAADDSYLEQAAAAVVAFVIRKGGSPMEAVIKAKEVVDRIKEAADSREATRKAARMVATVIQAGGSPEEAVKDAKKLVDLLRALRELEKALRELKKSLDELERSLEELEKNPSEDALVENNRLNVENNKIIVEVLRIIAEVLKINAELVGSWSGGGSEQKLISEEDLGGS

SEQ ID NO: 49 - protein, 279 aa, synthetic construct
  UNSURE 1..29: note = optionally absent
MHHHHHHGGSEQKLISEEDLSGGGSWSGSPELEEWIRRAKEVAKEVEKVAQRAEEEGNPDLRDSAKELRKAVELAILIAKMLGNPELVEWVARAAKVAAEVIKVAIQAEKEGNRDLFRAALELVRAVIEAINIAVVLGDPRLVEAVARAAKVAAEVIKVAILAEKMGAREMFRKALELVRKVIEAIETAVIEGDPEKVERVAREATKEALKILLWLLEKLLRELKKSLDELERSLEELEKNPSEDALVENNRLNVENNKIIVKVLEMIARVLKMNAKAV

SEQ ID NO: 50 - protein, 301 aa, synthetic construct
  UNSURE 1..51: note = optionally absent
METDTLLLWVLLLWVPGSTGDGSHHHHHHGGSEQKLISEEDLSGGGSWSGSPELEEWIRRAKEVAKEVEKVAQRAEEEGNPDLRDSAKELRKAVELAILIAKMLGNPELVEWVARAAKVAAEVIKVAIQAEKEGNRDLFRAALELVRAVIEAINIAVVLGDPRLVEAVARAAKVAAEVIKVAILAEKMGAREMFRKALELVRKVIEAIETAVIEGDPEKVERVAREATKEALKILLWLLEKLLRELKKSLDELERSLEELEKNPSEDALVENNRLNVENNKIIVKVLEMIARVLKMNAKAV

SEQ ID NO: 51 - protein, 279 aa, synthetic construct
  UNSURE 1..29: note = optionally absent
MHHHHHHGGSEQKLISEEDLSGGGSWSGSPELEEWIRRAKEVAKEVEKVAQRAEEEGNPDLRDSAKELRKAVELAIEIARWLGNPELVEWVARAAKVAAEVIKVAIQAEKEGNRDLFRAALELVRAVIEAINIAVVLGDPRLVEAVARAAKVAAEVIKVAIDAEKAGAREMFRRALELVREVIEAIEEAVIEGDPERVERVARKATKEALDIALKLLEMLLQRLREMLRKLKESLEELKKNPSEDALVRNNELIVEVLRVIVEVLSMIAKVLKLNAKLV

SEQ ID NO: 52 - protein, 301 aa, synthetic construct
  UNSURE 1..51: note = optionally absent
METDTLLLWVLLLWVPGSTGDGSHHHHHHGGSEQKLISEEDLSGGGSWSGSPELEEWIRRAKEVAKEVEKVAQRAEEEGNPDLRDSAKELRKAVELAIEIARWLGNPELVEWVARAAKVAAEVIKVAIQAEKEGNRDLFRAALELVRAVIEAINIAVVLGDPRLVEAVARAAKVAAEVIKVAIDAEKAGAREMFRRALELVREVIEAIEEAVIEGDPERVERVARKATKEALDIALKLLEMLLQRLREMLRKLKESLEELKKNPSEDALVRNNELIVEVLRVIVEVLSMIAKVLKLNAKLV

SEQ ID NO: 53 - protein, 240 aa, synthetic construct; identical to SEQ ID NO: 43
  UNSURE 1..29: note = optionally absent

SEQ ID NO: 54 - protein, 262 aa, synthetic construct; identical to SEQ ID NO: 44
  UNSURE 1..51: note = optionally absent

SEQ ID NO: 55 - protein, 225 aa, synthetic construct
  UNSURE 1..29: note = optionally absent
MHHHHHHGGSEQKLISEEDLSGGGSWSGSSEKEKVEELAQRIREQLPDTRLALMAQALANLANALDDSEALKVVYLALRIVQQLPDTELAREALELALDAAKSTDSKALEVVKLALRIVQLLPDTEDAREALELAKEAVKSTDEEERKKVKIKLKLLEALAELEKALRELKKSLDELERSLEELEKNPSEDALVENNRLNVENNKIIVEVLRIIAEVLKINAKLA

SEQ ID NO: 56 - protein, 247 aa, synthetic construct
  UNSURE 1..51: note = optionally absent
METDTLLLWVLLLWVPGSTGDGSHHHHHHGGSEQKLISEEDLSGGGSWSGSSEKEKVEELAQRIREQLPDTRLALMAQALANLANALDDSEALKVVYLALRIVQQLPDTELAREALELALDAAKSTDSKALEVVKLALRIVQLLPDTEDAREALELAKEAVKSTDEEERKKVKIKLKLLEALAELEKALRELKKSLDELERSLEELEKNPSEDALVENNRLNVENNKIIVEVLRIIAEVLKINAKLA

SEQ ID NO: 57 - protein, 316 aa, synthetic construct
  UNSURE 1..29: note = optionally absent
MHHHHHHGGSEQKLISEEDLSGGGSWSGSPRERLEEAKERVEEIRELIDKARKLQEQGNRVDATAVLMEARSQIREVTRELEEIAKNSDTPELATRAAELLVRLIKLLIEIAKLLQEQGQTQSAEDVLREATELIKRVTELLEKIAKNSDTPELALRAAELLVRLIKLLIEIAKLLQEQGNKEEATKVLREAEELIERVFELLKKIAENSDTPELAKRAEELIERLIELLEEIAKLLEEAGRRKEALRVLLKALELLLRLLKKSLDELERSLEELEKNPSEDALVENNRLNVKNNRIIVKVLEMIAKVLKMNAKAV

SEQ ID NO: 58 - protein, 338 aa, synthetic construct
  UNSURE 1..51: note = optionally absent
METDTLLLWVLLLWVPGSTGDGSHHHHHHGGSEQKLISEEDLSGGGSWSGSPRERLEEAKERVEEIRELIDKARKLQEQGNRVDATAVLMEARSQIREVTRELEEIAKNSDTPELATRAAELLVRLIKLLIEIAKLLQEQGQTQSAEDVLREATELIKRVTELLEKIAKNSDTPELALRAAELLVRLIKLLIEIAKLLQEQGNKEEATKVLREAEELIERVFELLKKIAENSDTPELAKRAEELIERLIELLEEIAKLLEEAGRRKEALRVLLKALELLLRLLKKSLDELERSLEELEKNPSEDALVENNRLNVKNNRIIVKVLEMIAKVLKMNAKAV

SEQ ID NO: 59 - protein, 204 aa, synthetic construct
RFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 60 - protein, 204 aa, synthetic construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 61 - protein, 204 aa, synthetic construct
RFPNITNLCPFGEVFNATRFASVYAWNRKRISNCVADYSVLYNSASFSTFKCYGVSPTKLNDLCWTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 62 - protein, 203 aa, synthetic construct
RFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLST

SEQ ID NO: 63 - protein, 203 aa, synthetic construct
RFPNITNLCPFGEVFNATKFPSVYAWERKKISNCVADYSVLYNSTFFSTFKCYGVSATKLNDLCWSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLST

SEQ ID NO: 64 - protein, 203 aa, synthetic construct
RFPNITNLCPFGEVFNATKFPSVYAWERKKISNCVADFSVLYNSTFFSTFKCYGVSATKLNDLCWSNIYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLST

SEQ ID NO: 65 - protein, 203 aa, synthetic construct
RFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKST

SEQ ID NO: 66 - protein, 203 aa, synthetic construct
RFPNITNLCPFGQVFNASNFPSVYAWERLRISDCVADYAVLYNSSSSFSTFKCYGVSPTKLNDLCWSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKST

SEQ ID NO: 67 - protein, 203 aa, synthetic construct
RFPNITNLCPFGQVFNASNFPSVYAWERLRISDCVADFAVLYNSSSSFSTFKCYGVSPTKLNDLCWSSIYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKST

SEQ ID NO: 68 - protein, 185 aa, synthetic construct
RFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLST

SEQ ID NO: 69 - protein, 185 aa, synthetic construct
RFPNITNFCPFDKVFNATRFPNVYAWQRTKISDCIADYTVLYNSTSFSTFKCYGVSPSKLIDLCWTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLST

SEQ ID NO: 70 - protein, 185 aa, synthetic construct
RFPNITNFCPFDKVFNATRFPNVYAWQRTKISDCIADFTVLYNSTSFSTFKCYGVSPSKLIDLCWTSIYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLST

SEQ ID NO: 71 - protein, 239 aa, synthetic construct
  UNSURE 1..23: note = optionally absent
  UNSURE 228..239: note = optionally absent
METDTLLLWVLLLWVPGSTGDGSKMEELFKEHKIVAVLRANSVEEAKKKALAVFLGGVDLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLDNVAEWFEAGVQAVGVGEALNEGTPVEVAEKAKAFVEKIEGATEGSEQKLISEEDL

SEQ ID NO: 72 - protein, 467 aa, synthetic construct
  UNSURE 1..32: note = optionally absent
  UNSURE 237..263: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKEHKIVAVLRANSVEEAKKKALAVFLGGVDLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLDNVAEWFEAGVQAVGVGEALNEGTPVEVAEKAKAFVEKIEGATE

SEQ ID NO: 73 - DNA, 1866 bp, synthetic construct
  source 1..1866: mol_type = other DNA
  unsure 1..54: note = optionally absent
  unsure 1456..1866: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctccgagaaggccgccaaggccgaggaggccgccaggaagatggaggagctctttaaggagcacaaaatcgtggcggtgctcagggcaaatagcgtagaagaggccaaaaagaaggcactcgccgtctttctcggaggcgtggacctgatagagatcacattcacggtgccagatgctgatacggtcattaaggagctgagttttctcaaagagatgggcgccatcataggcgccggcaccgtgacatccgtggagcaggccagagaagccgtcgagtccggagccgaattcatagtgtcccctcatctcgatgaggagatcagtcagtttgccaaggaggagggagtgttttatatgcccggagtgatgacgccgacggagctagtgaaggccatgaaactgggccacaccatcctcaagctgtttcctggagaagtggttggtccccaatttgtcgaagccatgaagggaccatttccaaacgtgaaattcgtgccgaccggaggagttaacttagacaacgttgccgagtggtttgaggcaggcgttcaggcggtcggggtcggagaggccctcaacgaaggaacaccagtggaagtcgccgagaaggccaaagcatttgtggagaaaatcgagggagccactgaatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 74 - protein, 487 aa, synthetic construct
  UNSURE 1..32: note = optionally absent
  UNSURE 237..252: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSSDEEEAREWAERALKAALEAAEQALREGDEDAFKCAVELLEQALEARKKKDSEEAEAVYWAARAVLAALEALEQAKREGDEDARRCAEELLRLACEAARKKNSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 75 - DNA, 1926 bp, synthetic construct
  source 1..1926: mol_type = other DNA
  unsure 1..54: note = optionally absent
  unsure 1516..1926: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctccagtgatgaggaggaagctcgcgaatgggctgaacgggcactcaaagccgctctggaggccgcagaacaagcactgcgcgaaggagacgaggacgcttttaaatgcgctgttgaacttctggaacaggccctcgaagctcggaagaaaaaggatagcgaggaagctgaagccgtctattgggcagctagagctgtcctcgccgcgctggaagctctggaacaagcaaaacgcgagggagatgaggatgctagacgctgtgccgaggaattgctgaggctggcctgcgaggccgcgagaaagaagaacagcgaacaggctagggccgtctatgaagcagctagggctgttttggccgcactgagggctctggaagccgcaaaacgcgccggaatggaggaagcacgcaaggaagccgaggagttgctccgaagagcatgcgaagccgcccgcaagcaggatcctgagctcgctagagcagttcgggataaagctgaactcctcaaagctctcgccgacttgttcaaggccctcaaggaacttaagaagtcccttgacgagctcgagcggtccctcgaagagctggagaagaaccccagcgaggatgctctggtcgagaataatcggcttaacgtggagaacaataagattatcgtcgaagtgctccgcattatcgctgaagtgctccgaattaatgcacgcgccgtttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 76 - protein, 478 aa, synthetic construct
  UNSURE 1..32: note = optionally absent
  UNSURE 237..252: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSTEVEKKAREVAKEAVELASLLRSETAIRVAQAILEAAEAAKRAAEQGKTEVAKLALKVLEEAIELAKEKRSEEALKVVLEIARAALAAAQAAEEGFTDVAKMALEVLERAIELAKDDRSEEALKEVLEIARAALAAAQLAKKGRDDEARKILMKLRIRITLRKLEESLRELRRILEELKEMLERLEKNPDKDVIVKVLKVIVKAIEASVENQRISAENQKALAELA

SEQ ID NO: 77 - DNA, 1899 bp, synthetic construct
  source 1..1899: mol_type = other DNA
  unsure 1..54: note = optionally absent
  unsure 1489..1899: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctccactgaagtcgagaagaaggccagagaggtcgctaaggaagcggttgagctggcatctcttctgcgtagcgaaacagcaattcgagtagcccaagctatccttgaagccgccgaggcagccaaacgggcagctgaacaggggaagaccgaagttgcaaaactcgccctgaaggtactcgaggaagccattgaactggctaaagaaaaacggagcgaggaagccctcaaagttgttctggaaatcgccagggctgcactggccgccgcacaagcagctgaggaagggttcactgatgtcgctaaaatggccctcgaggtcctggaaagagcaatagagctggcaaaggatgaccgctccgaagaggctctgaaggaagttctggagatcgccagagcagccctcgctgcggcgcagctggccaaaaaaggccgcgatgatgaagctcggaaaattctgatgaaactgaggattcgcatcacactgcggaaattggaggagtcacttcgcgagttgcggaggatccttgaggagctcaaagaaatgcttgagcgcttggagaaaaatcctgacaaagacgtcattgtaaaggtcctcaaagtgattgtcaaggctattgaggcctccgtggagaatcaacgcatttccgctgagaatcagaaggcccttgctgaattggcatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 78 - protein, 507 aa, synthetic construct
  UNSURE 1..32: note = optionally absent
  UNSURE 237..252: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSTEEKIEEARQAIKEAERALREGDPRFAEMAVRIALALVRMLERLARKTGSTEVLIEAARLAIEVARVALKVGSPETAREAVRTALELVQELERQARKTGSTEVLIEAARLAIEVARVAFKVGSPETAKEAVRTALELVKELIQQALKTGSDEVLERAAELAKEVARVAKEVGDPRAARKADMVAKIADTLRELMESLRELRRILEELKEMLERLEKNPDKDVIVKVLKVIVKAIEASVENQRISAENQAALASLA

SEQ ID NO: 79 - DNA, 1986 bp, synthetic construct
  source 1..1986: mol_type = other DNA
  unsure 1..54: note = optionally absent
  unsure 1576..1986: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctccaccgaggagaaaatcgaagaggcacggcaggcaatcaaggaggctgaacgggcactgcgcgaaggagaccctcgatttgctgaaatggctgtcagaatcgcccttgccttggttcggatgctggaacgcctcgcccgcaagaccggatccactgaggttctcattgaggcagcaaggcttgctattgaggtggctcgggtggccctcaaggtcggttcgccggagaccgctagagaagcagtcagaacagcccttgaactggtccaagagttggagcggcaagcacgcaagaccgggtctactgaggtgctgattgaagctgccaggctcgctattgaggtcgctagggtcgctttcaaagttggttctcctgagaccgctaaggaagctgttagaacagctctggaactggttaaggaactgatccaacaagcgcttaagaccggctctgacgaagttctggagcgggctgctgaacttgctaaggaagttgccagggtggccaaagaggttggcgatcctcgggctgcccgcaaggcagatatggtggccaagatcgccgatacactccgcgagcttatggaatccctgcgcgagctcaggcggatccttgaggagctcaaggaaatgctggagagactcgagaagaatcccgataaagacgtgatcgtcaaagtgctgaaagtgatcgttaaggctattgaagcaagcgttgagaaccagcgcatttctgctgagaatcaagcagctcttgctagtctggcttgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 80 - protein, 539 aa, synthetic construct
  UNSURE 1..32: note = optionally absent
  UNSURE 320..335: note = optionally absent
MGILPSPGMPALLSLVSLLSVLLMGCVAETGTADELRAVAELQRLNIELARKLLEAVARLQELNIDLVRKTSELTDEKTIREEIRKVKEESKRIVEEAEEEIRRAKEDSKRIVTEALRRAREQIREKWEELEERAKRAETPEEALRAAEEMVKLIEELIRIAEMLQRAGLKEEAEDVLREATELIKRATELLEKIAKNSDTPELALRAAELLVRLIKLLIEIAKLLQEQGNKEEAEKVLREATELIKRVARLLLAIALLADTPELAKRAAELLKRLIELLKEIAKLLEEEGNEDEAEKVKEEAKELEELVRWLEEQIRGGGSGGSGSGGSGGSGSRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 81 - DNA, 2082 bp, synthetic construct
  source 1..2082: mol_type = other DNA
  unsure 1..54: note = optionally absent
  unsure 1651..2082: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacagccgatgagctacgcgctgtagctgagctgcagcgacttaacattgaactggcacgcaaacttctcgaggccgtggccaggctgcaggagctcaacatcgatctggtccgcaagacaagcgaattgacagacgagaagaccatacgcgaggagatcagaaaagtcaaggaggaatctaaacgcattgtggaagaggcagaggaagagattaggcgcgcaaaggaggacagcaaaagaatcgtgaccgaggcactcaggcgggccagggaacagataagagagaaatgggaagagcttgaggagcgggcaaagcgagccgaaacacccgaagaggcattacgagcggccgaggagatggttaagcttattgaagagctgattcgaatcgcagaaatgttgcaaagggcggggctcaaggaggaggccgaagacgtcctccgcgaggccacagaactcatcaaacgggccaccgaacttctcgagaaaatcgctaagaacagcgatacaccggaactggcacttcgggccgccgaacttttggttcgtttaatcaagcttttgatcgaaatcgcaaaacttctccaagaacagggcaacaaggaggaggcagagaaagtccttcgcgaggcaaccgaacttatcaaaagggtcgccagactgctcctggccatcgcactgctggcggatacgccagaacttgcaaagcgcgccgccgagctgctgaaacgattgattgaattgctcaaggagatcgccaaactcctggaggaggaaggtaacgaggatgaggcagagaaagtgaaggaagaggccaaggaactcgaggagctggtccgttggctggaggaacaaatccgaggtggaggctctggaggcagcggctccggaggctctggaggcagcggctccaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 82 - protein, 487 aa, synthetic construct
  UNSURE 1..32: note = optionally absent
  UNSURE 237..252: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSSDEEEAREWAERAEEAAKEALEQAKREGDEIARLCAKMLEILAEEARRKKDSEEAEAVYWAARAVLAALEALEQAKREGDEDARRCAEELLRLACSAAARQDSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 83 - DNA, 1926 bp, synthetic construct
  source 1..1926: mol_type = other DNA
  unsure 1..54: note = optionally absent
  unsure 1516..1926: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctccagcgacgaggaggaagctcgcgaatgggccgagcgcgctgaggaagctgcaaaggaggctctcgagcaagcaaagagggaaggcgacgaaatcgcacggttatgcgctaaaatgctggaaatcctggccgaagaggcccgccgcaaaaaggattctgaggaggctgaagctgtctattgggccgctagagctgttctggcggcattggaggcactggagcaggctaaacgcgaaggcgacgaggatgctaggcgctgtgctgaggaactcctgagacttgcctgttctgccgccgctcgccaagattcagagcaagcccgggctgtctacgaggcagctcgagctgttctggccgccctgagagctctcgaagcagcaaaacgggcaggcatggaggaggctcgcaaggaagctgaggagcttcttcgccgagcatgcgaagccgcccgcaaacaggatcccgagctggcaagagccgtcagagataaggccgagttgcttaaggcacttgctgacctatttaaagccctgaaggagctcaagaagtcccttgacgaactggaaaggagcctcgaagagctcgagaagaatccttccgaggatgcactggttgagaacaatagattgaacgtggaaaacaataagattatcgttgaggtgcttaggattatcgccgaggtgctcagaattaatgctagagccgtctgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 84 - protein, 503 aa, synthetic construct
  UNSURE 1..32: note = optionally absent
  UNSURE 284..299: note = optionally absent
MGILPSPGMPALLSLVSLLSVLLMGCVAETGTDESVDLAVKLAEALRKEAEELIKKARKTGDPELLRKALEALEKAVKLVEDAIKRNPDNDEAVETAVRLARELKKVAEELQERAKKTGDPELLKLALRALEVAVRAVELAIKSNPDNDEAVKTAVELAKELEKVARELLERARKTGDDELLKLAKRALEVARRAVELALKSRPDAEEARRVYIRLTEMELEISLTELRKILEELKEMLERLEKNPDKDVIVKVLKVIVKAIEASVENQRISAENQKALAELAGGSGGSGSGGSGGSGSRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 85 - DNA, 1974 bp, synthetic construct
  source 1..1974: mol_type = other DNA
  unsure 1..54: note = optionally absent
  unsure 1564..1974: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacagatgagtctgttgacctcgctgtaaagctagccgaagccctccgcaaggaggccgaggagctgataaagaaggctcggaagacaggcgaccccgaactgcttaggaaggctctcgaggccctcgagaaagctgtgaagcttgtcgaggacgccatcaaacggaatcccgacaatgacgaggccgtggaaactgcagttaggctcgctcgggaattgaagaaggtcgccgaggagctgcaggaacgggccaaaaagactggtgaccccgagctcctcaaactcgctctgagagccctggaggtggcagtccgcgccgtagaactggccatcaagtctaatcctgataacgatgaggcagttaagactgctgtggagcttgcaaaggagctcgagaaagtggcacgcgaactgttggagagagctcgcaagactggggatgacgaactcctgaagctggccaagcgcgctcttgaagtggcaaggcgcgccgtggaacttgcccttaagagccggcccgatgcggaggaggcaagacgggtctacattcgcctgaccgagatggagcttgagatttccctgaccgaactccgcaaaattctcgaggagctcaaggaaatgttggaacggcttgagaagaacccagataaggacgtcatcgttaaggtgctcaaggttattgttaaagctattgaagcatccgtcgaaaaccagagaatctcagccgagaaccagaaagcccttgctgaactggcaggaggctctggaggcagcggctccggaggctctggaggcagcggctccaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 86 - protein, 507 aa, synthetic construct
  UNSURE 1..32: note = optionally absent
  UNSURE 237..252: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSTEETIEMARQLIKEAERALREGDPEEARMAVEMALAAVRILERQARKTGSTEVLIEAARLAIEVARVALKVGSPETAREAVRTALELVQELERQARKTGSTEVLIEAARLAIEVARVAFKVGSPETAKEAVRTALELVKELIQQALKTGSDEVLERAAELAKEVARVAKEVGDPRAARKADMVAKIADTLRELMESLRELRRILEELKEMLERLEKNPDKDVIVKVLKVIVKAIEASVENQRISAENQAALASLA

SEQ ID NO: 87 - DNA, 1986 bp, synthetic construct
  source 1..1986: mol_type = other DNA
  unsure 1..54: note = optionally absent
  unsure 1576..1986: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctccactgaggagacaatcgaaatggctagacaactcataaaggaagctgagcgcgctctgagagaaggggaccccgaggaggccagaatggccgtggaaatggctttggccgctgtccgtatactcgagcgccaagctaggaagaccggttccaccgaagtcctgattgaggctgctaggctggcaatcgaggttgccagggtggcactgaaagtcggcagccccgagacagccagagaagctgtcaggacagcacttgaacttgtccaagagttggaacgccaggccaggaaaactgggtctaccgaggtgctgattgaggccgcacggctggctattgaggtcgctagggtggctttcaaagtcggcagtcctgaaactgcaaaggaagcagttcgcaccgcattagaacttgttaaggagctgatacaacaggcactcaagaccggcagtgacgaggttcttgagcgggctgcagagcttgctaaagaggtggcaagggtggctaaggaagttggagatccacgggccgctcgcaaagcagatatggttgccaaaattgccgataccctccgcgaacttatggagtccctgagggaactgcgccgaattctcgaggaacttaaggaaatgctggagcggctcgagaagaaccctgacaaggatgtgattgtgaaggttcttaaagtgatcgttaaagccatcgaagcctctgtcgaaaaccaacgaatctccgctgagaaccaagctgctttggcttcattggcgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 88 - protein, 531 aa, synthetic construct
  UNSURE 1..32: note = optionally absent
  UNSURE 237..252: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSGRELKQLAEVLEEIQRLAEEARKLMTDEEEAKKIQEEAERAKRMLASAVWAVTDNEVIEKLLEVVKEIIRLAEEAMKKMTDEEEAAKIAKEALEAIKMLARAVEEVTDNEVIEKLLEVVKEIIRAAEEAMKLMRNEEEAAKIAKKALEAIKALAEAVEAIKNKEEIERLLTLVKELIRKAEEEARRMSDREKAAEIIERALEKIKKLAKLAKALADLEKALRELKKSLDELERSLEELERNPSEAALVENNRLNVENNKIIVEVLRIIAEVLKINAELA

SEQ ID NO: 89 - DNA, 2058 bp, synthetic construct
  source 1..2058: mol_type = other DNA
  unsure 1..54: note = optionally absent
  unsure 1648..2058: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctccggccgcgaactgaaacagcttgctgaggtgctcgaggaaattcaacgcctggctgaggaagcccgcaagctgatgacagacgaggaggaagccaagaagattcaggaggaggccgagcgcgctaagcgcatgctggcatccgctgtttgggccgtaacggataacgaagtgattgagaagctcctcgaggtcgtgaaagagatcatccgcctcgccgaggaggctatgaagaagatgaccgacgaggaggaagctgccaagatcgcaaaggaggccctcgaggccatcaaaatgctggctagagccgttgaggaagtgacagataatgaagtaattgagaagctgctcgaggtggtcaaagagattatcagggccgccgaggaggccatgaagctcatgagaaacgaagaagaggcagctaagatcgctaagaaggctctggaggccattaaagctctcgctgaggccgtggaggctatcaagaacaaggaggagattgagcggcttctgactcttgtcaaggagctgatccggaaagctgaggaggaagcaaggcggatgagcgaccgcgagaaagccgctgagatcattgagcgggcactcgagaaaattaaaaagcttgcaaagctggctaaagccctggccgatcttgagaaagccctgagggaactcaaaaagtccctcgacgaactggagcggagtctggaagagctcgagcgcaacccttctgaggccgccctggttgagaacaaccgcctgaatgtcgagaacaataagatcatcgtcgaggttctccggatcattgctgaggtgctcaaaattaacgccgaactcgcgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 90 - protein, 487 aa, synthetic construct
  UNSURE 1..32: note = optionally absent
  UNSURE 237..252: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSSDEEEAREWAERAEEAAKEALEQAKREGDEIARLCAEMLEILAEEARRKKDSEEAEAVYWAARATLAALEALEQAKREGDEDARRCAEELLRLACSAAARQDSEQARAVYEAARAVLAALRALEAAKRAGMEEARKEAEELLRRACEAARKQDPELARAVRDKAELLKALADLFKALKELKKSLDELERSLEELEKNPSEDALVENNRLNVENNKIIVEVLRIIAEVLRINARAV

SEQ ID NO: 91 - DNA, 1926 bp, synthetic construct
  source 1..1926: mol_type = other DNA
  unsure 1..54: note = optionally absent
  unsure 1516..1926: note = optionally absent
aggaataaactagtattcttctggtccccacagactcagagagaacccgccaccatgggcatcctgccaagccctggaatgcctgccctgctgagcctggtgtccctgctgtctgtgctgctgatgggatgcgtggcagagaccggcacaaggttccctaacatcaccaatctgtgcccattcggcgaggtgtttaacgccacacgctttgcctccgtgtatgcctggaaccggaagagaatctctaattgcgtggccgacttcagcgtgctgtacaatagcgcctccttctctacctttaagtgctatggcgtgagccccaccaagctgaacgatctgtgctggacaaacatttacgccgactcctttgtgatccggggcgatgaagtgagacagatcgcaccaggacagaccggaaagatcgcagactacaactataagctgcctgacgatttcacaggctgcgtgatcgcctggaatagcaacaatctggattccaaagtgggcggcaactacaattatctgtacaggctgttccgcaagtccaacctgaagccatttgagcgggacatctccaccgagatctaccaggccggctctacaccctgcaacggcgtggagggcttcaattgttattttcccctgcagtcttacggcttccagcctacaaatggcgtgggctatcagccataccgggtggtggtgctgtcttttgagctgctgcacgcaccagcaaccgtgtgcggccccaagaagagcacaggaggctctggaggcagcggctccggaggctctggaggcagcggctcctccgacgaagaggaagctagagaatgggccgaaagagcagaagaggcagccaaggaggcattggaacaagctaagcgcgaaggggatgaaatcgctagactttgcgctgaaatgcttgagatcctggccgaagaggctaggcgcaagaaggactctgaggaggccgaggcagtgtattgggccgctagagccaccctggctgctctggaagcactggaacaggctaaacgcgagggcgatgaggacgctaggcgctgtgctgaggaactccttagactcgcctgtagtgcagccgcacggcaggactctgagcaagcaagggcagtttatgaggctgctagggctgttttggccgccctgcgggctcttgaggctgctaaacgggctgggatggaggaagcccgcaaggaagctgaggaactgctgagacgcgcctgtgaagctgctagaaagcaggatcccgaacttgcgagggctgttcgcgataaagcagagcttctgaaggcactggccgatctcttcaaggctctcaaggaactcaaaaagtctctggatgagctcgaacgatctctggaggagctggagaagaatccttccgaggacgcactcgtcgagaacaatagactgaatgttgagaataacaagatcattgttgaagttctacggattatcgctgaggtcttgcgcattaatgcaagagccgtgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa